SEQUENCE LISTING (684 sequences)

SEQ ID NO: 1 - DNA, 19 bp, Artificial sequence
  Synthetic oligonucleotide
catccatgaaagcgaaagg

SEQ ID NO: 2 - DNA, 19 bp, Artificial sequence
  Synthetic oligonucleotide
caggttcccacctgttctg

SEQ ID NO: 3 - DNA, 18 bp, Artificial sequence
  Synthetic oligonucleotide
agtcccgggtaaggcaag

SEQ ID NO: 4 - DNA, 18 bp, Artificial sequence
  Synthetic oligonucleotide
tcaaggccatcgagctgt

SEQ ID NO: 5 - DNA, 18 bp, Artificial sequence
  Synthetic oligonucleotide
tccctgaagcctccctaa

SEQ ID NO: 6 - DNA, 18 bp, Artificial sequence
  Synthetic oligonucleotide
aatcacgcccatggaacc

SEQ ID NO: 7 - DNA, 18 bp, Artificial sequence
  Synthetic oligonucleotide
ccgtcccttcctccattc

SEQ ID NO: 8 - DNA, 18 bp, Artificial sequence
  Synthetic oligonucleotide
gacaccatctcggccatt

SEQ ID NO: 9 - DNA, 18 bp, Artificial sequence
  Synthetic oligonucleotide
acgcacgtcgagaatgtg

SEQ ID NO: 10 - DNA, 18 bp, Artificial sequence
  Synthetic oligonucleotide
gcataattgcgggtcagg

SEQ ID NO: 11 - DNA, 18 bp, Artificial sequence
  Synthetic oligonucleotide
cccatgcattctggtgga

SEQ ID NO: 12 - DNA, 18 bp, Artificial sequence
  Synthetic oligonucleotide
gcgtgtcctcgcaaaaag

SEQ ID NO: 13 - DNA, 18 bp, Artificial sequence
  Synthetic oligonucleotide
cgacggtgacctttgagg

SEQ ID NO: 14 - DNA, 18 bp, Artificial sequence
  Synthetic oligonucleotide
caagatggcgggcatagt

SEQ ID NO: 15 - DNA, 18 bp, Artificial sequence
  Synthetic oligonucleotide
ggcgtggactttgtgtcc

SEQ ID NO: 16 - DNA, 18 bp, Artificial sequence
  Synthetic oligonucleotide
gggtacgtggcagtctgg

SEQ ID NO: 17 - DNA, 18 bp, Artificial sequence
  Synthetic oligonucleotide
ggcctgtctggttgagga

SEQ ID NO: 18 - DNA, 19 bp, Artificial sequence
  Synthetic oligonucleotide
ctgtccccatggtccttag

SEQ ID NO: 19 - DNA, 18 bp, Artificial sequence
  Synthetic oligonucleotide
gctgcactacccccaatg

SEQ ID NO: 20 - DNA, 18 bp, Artificial sequence
  Synthetic oligonucleotide
tcttggtgaggggaccac

SEQ ID NO: 21 - DNA, 18 bp, Artificial sequence
  Synthetic oligonucleotide
gcacttggcgctgtaggt

SEQ ID NO: 22 - DNA, 18 bp, Artificial sequence
  Synthetic oligonucleotide
tgacgtccgtcgctaaga

SEQ ID NO: 23 - DNA, 18 bp, Artificial sequence
  Synthetic oligonucleotide
tcgtcctcctcctcgtca

SEQ ID NO: 24 - DNA, 20 bp, Artificial sequence
  Synthetic oligonucleotide
tggaaatggattggtcacct

SEQ ID NO: 25 - DNA, 18 bp, Artificial sequence
  Synthetic oligonucleotide
tgcccagacacaccactg

SEQ ID NO: 26 - DNA, 18 bp, Artificial sequence
  Synthetic oligonucleotide
actcggcttttgcgactg

SEQ ID NO: 27 - DNA, 18 bp, Artificial sequence
  Synthetic oligonucleotide
ccatcggcgagcttttta

SEQ ID NO: 28 - DNA, 19 bp, Artificial sequence
  Synthetic oligonucleotide
gcagtgctacccccatttt

SEQ ID NO: 29 - DNA, 18 bp, Artificial sequence
  Synthetic oligonucleotide
tctgcgccattcaaaaca

SEQ ID NO: 30 - DNA, 18 bp, Artificial sequence
  Synthetic oligonucleotide
tccagaatgcgcagatca

SEQ ID NO: 31 - DNA, 19 bp, Artificial sequence
  Synthetic oligonucleotide
ccacaggcttgtgcagact

SEQ ID NO: 32 - DNA, 19 bp, Artificial sequence
  Synthetic oligonucleotide
tgtacgtgtcggtgcgtta

SEQ ID NO: 33 - DNA, 19 bp, Artificial sequence
  Synthetic oligonucleotide
ttggagcatcttccacagg

SEQ ID NO: 34 - DNA, 18 bp, Artificial sequence
  Synthetic oligonucleotide
tctgtctccgtcgcaggt

SEQ ID NO: 35 - DNA, 18 bp, Artificial sequence
  Synthetic oligonucleotide
cagatcctcgcgcaaacc

SEQ ID NO: 36 - DNA, 20 bp, Artificial sequence
  Synthetic oligonucleotide
tggagcttctgacgaagacc

SEQ ID NO: 37 - DNA, 18 bp, Artificial sequence
  Synthetic oligonucleotide
gggcgatggggcttatag

SEQ ID NO: 38 - DNA, 18 bp, Artificial sequence
  Synthetic oligonucleotide
gcagctgtcggtgaggac

SEQ ID NO: 39 - DNA, 18 bp, Artificial sequence
  Synthetic oligonucleotide
gcttggcaatgcagtcgt

SEQ ID NO: 40 - DNA, 18 bp, Artificial sequence
  Synthetic oligonucleotide
gcccagggccttaagttt

SEQ ID NO: 41 - DNA, 21 bp, Artificial sequence
  Synthetic oligonucleotide
tggttgagtccattctccttg

SEQ ID NO: 42 - DNA, 18 bp, Artificial sequence
  Synthetic oligonucleotide
gcagcaggttgcacttca

SEQ ID NO: 43 - DNA, 21 bp, Artificial sequence
  Synthetic oligonucleotide
tgaagcgggctttaatactga

SEQ ID NO: 44 - DNA, 18 bp, Artificial sequence
  Synthetic oligonucleotide
cgttcgactggcaggagt

SEQ ID NO: 45 - DNA, 19 bp, Artificial sequence
  Synthetic oligonucleotide
ccagcgtgactcaggagat

SEQ ID NO: 46 - DNA, 18 bp, Artificial sequence
  Synthetic oligonucleotide
gacggccgaatctcactg

SEQ ID NO: 47 - DNA, 20 bp, Artificial sequence
  Synthetic oligonucleotide
ggccccttaaagatcacctg

SEQ ID NO: 48 - DNA, 23 bp, Artificial sequence
  Synthetic oligonucleotide
tggaccagaacaatctttagtgc

SEQ ID NO: 49 - DNA, 19 bp, Artificial sequence
  Synthetic oligonucleotide
gcctacagacggtccaaag

SEQ ID NO: 50 - DNA, 18 bp, Artificial sequence
  Synthetic oligonucleotide
tccgagggtatgccagag

SEQ ID NO: 51 - DNA, 18 bp, Artificial sequence
  Synthetic oligonucleotide
ttttggatgcccacaagg

SEQ ID NO: 52 - DNA, 19 bp, Artificial sequence
  Synthetic oligonucleotide
gagctgtgtgcgagggata

SEQ ID NO: 53 - DNA, 18 bp, Artificial sequence
  Synthetic oligonucleotide
gcggcagactccttttcc

SEQ ID NO: 54 - DNA, 18 bp, Artificial sequence
  Synthetic oligonucleotide
gcgaggataatggggaca

SEQ ID NO: 55 - DNA, 22 bp, Artificial sequence
  Synthetic oligonucleotide
gggaaacaaaaacatcaacact

SEQ ID NO: 56 - DNA, 18 bp, Artificial sequence
  Synthetic oligonucleotide
accatcgccaccactcct

SEQ ID NO: 57 - DNA, 18 bp, Artificial sequence
  Synthetic oligonucleotide
tggctgtgcacacaaggt

SEQ ID NO: 58 - DNA, 18 bp, Artificial sequence
  Synthetic oligonucleotide
gcccgctgctatttttca

SEQ ID NO: 59 - DNA, 18 bp, Artificial sequence
  Synthetic oligonucleotide
aatagcgtgccccagttg

SEQ ID NO: 60 - DNA, 26 bp, Artificial sequence
  Synthetic oligonucleotide
ggattggatagtatgtcaagtcaaca

SEQ ID NO: 61 - DNA, 18 bp, Artificial sequence
  Synthetic oligonucleotide
tccgcaggatacccactc

SEQ ID NO: 62 - DNA, 18 bp, Artificial sequence
  Synthetic oligonucleotide
acagtgcagcggatgtca

SEQ ID NO: 63 - DNA, 18 bp, Artificial sequence
  Synthetic oligonucleotide
ggggcccgtttattatgg

SEQ ID NO: 64 - DNA, 18 bp, Artificial sequence
  Synthetic oligonucleotide
gcgttacatgggggacaa

SEQ ID NO: 65 - DNA, 18 bp, Artificial sequence
  Synthetic oligonucleotide
ttcgggaggctcaaagaa

SEQ ID NO: 66 - DNA, 18 bp, Artificial sequence
  Synthetic oligonucleotide
accggaagggctcagagt

SEQ ID NO: 67 - DNA, 18 bp, Artificial sequence
  Synthetic oligonucleotide
agaccccgaggctgatgt

SEQ ID NO: 68 - DNA, 18 bp, Artificial sequence
  Synthetic oligonucleotide
agccgtcctgtccaagtg

SEQ ID NO: 69 - DNA, 18 bp, Artificial sequence
  Synthetic oligonucleotide
tgactggcctcagcccta

SEQ ID NO: 70 - DNA, 18 bp, Artificial sequence
  Synthetic oligonucleotide
cctgactgaagcccagga

SEQ ID NO: 71 - DNA, 18 bp, Artificial sequence
  Synthetic oligonucleotide
tggtggcaggaatcatca

SEQ ID NO: 72 - DNA, 18 bp, Artificial sequence
  Synthetic oligonucleotide
ctgtggccctctgcaagt

SEQ ID NO: 73 - DNA, 18 bp, Artificial sequence
  Synthetic oligonucleotide
tggaaagcatcgtggtca

SEQ ID NO: 74 - DNA, 18 bp, Artificial sequence
  Synthetic oligonucleotide
tgtcggacgaggaggaag

SEQ ID NO: 75 - DNA, 18 bp, Artificial sequence
  Synthetic oligonucleotide
ctttgggaaagcgagctg

SEQ ID NO: 76 - DNA, 18 bp, Artificial sequence
  Synthetic oligonucleotide
tattcgagcccctcgttg

SEQ ID NO: 77 - DNA, 18 bp, Artificial sequence
  Synthetic oligonucleotide
gctgtctgccaccaggtc

SEQ ID NO: 78 - DNA, 18 bp, Artificial sequence
  Synthetic oligonucleotide
tgcaggccgacaggtagt

SEQ ID NO: 79 - DNA, 18 bp, Artificial sequence
  Synthetic oligonucleotide
cttcgtgcacaccaagga

SEQ ID NO: 80 - DNA, 18 bp, Artificial sequence
  Synthetic oligonucleotide
cccagccgcaaatatcag

SEQ ID NO: 81 - DNA, 18 bp, Artificial sequence
  Synthetic oligonucleotide
gcgagtagggccaggaac

SEQ ID NO: 82 - DNA, 18 bp, Artificial sequence
  Synthetic oligonucleotide
cgggggcataacactgag

SEQ ID NO: 83 - DNA, 18 bp, Artificial sequence
  Synthetic oligonucleotide
gccaaagaccaggctcaa

SEQ ID NO: 84 - DNA, 19 bp, Artificial sequence
  Synthetic oligonucleotide
cccttgccaccattctttt

SEQ ID NO: 85 - DNA, 18 bp, Artificial sequence
  Synthetic oligonucleotide
atgcgcaagggtcacatt

SEQ ID NO: 86 - DNA, 18 bp, Artificial sequence
  Synthetic oligonucleotide
gcgtcagcccatcttttg

SEQ ID NO: 87 - DNA, 19 bp, Artificial sequence
  Synthetic oligonucleotide
gccacaggtacaggcttga

SEQ ID NO: 88 - DNA, 18 bp, Artificial sequence
  Synthetic oligonucleotide
agcgggtgcagtaacagg

SEQ ID NO: 89 - DNA, 18 bp, Artificial sequence
  Synthetic oligonucleotide
agccgcttacagctcgac

SEQ ID NO: 90 - DNA, 18 bp, Artificial sequence
  Synthetic oligonucleotide
tggaaagcatcgtggtca

SEQ ID NO: 91 - DNA, 19 bp, Artificial sequence
  Synthetic oligonucleotide
ggacctacgctgccctaga

SEQ ID NO: 92 - DNA, 18 bp, Artificial sequence
  Synthetic oligonucleotide
ctaccagagggggccaag

SEQ ID NO: 93 - DNA, 18 bp, Artificial sequence
  Synthetic oligonucleotide
ggtgcgcctaggttttga

SEQ ID NO: 94 - DNA, 18 bp, Artificial sequence
  Synthetic oligonucleotide
cgccactagcagcaggtt

SEQ ID NO: 95 - DNA, 18 bp, Artificial sequence
  Synthetic oligonucleotide
ccgtgttttccccaacaa

SEQ ID NO: 96 - DNA, 18 bp, Artificial sequence
  Synthetic oligonucleotide
cttgtctatggcgcgttg

SEQ ID NO: 97 - DNA, 18 bp, Artificial sequence
  Synthetic oligonucleotide
tgacaccatccccgtctc

SEQ ID NO: 98 - DNA, 18 bp, Artificial sequence
  Synthetic oligonucleotide
ccggtgcatctggaagaa

SEQ ID NO: 99 - DNA, 18 bp, Artificial sequence
  Synthetic oligonucleotide
cacagcgtcaggggagac

SEQ ID NO: 100 - DNA, 18 bp, Artificial sequence
  Synthetic oligonucleotide
gcctgaggcatacccaca

SEQ ID NO: 101 - DNA, 18 bp, Artificial sequence
  Synthetic oligonucleotide
aatccgtcagcagcgtgt

SEQ ID NO: 102 - DNA, 18 bp, Artificial sequence
  Synthetic oligonucleotide
tgcaatttttgggccatt

SEQ ID NO: 103 - DNA, 19 bp, Artificial sequence
  Synthetic oligonucleotide
catgccatcctgggatatt

SEQ ID NO: 104 - DNA, 18 bp, Artificial sequence
  Synthetic oligonucleotide
ggcgacccaagttccttc

SEQ ID NO: 105 - DNA, 18 bp, Artificial sequence
  Synthetic oligonucleotide
ccctcagatgggtcttcg

SEQ ID NO: 106 - DNA, 18 bp, Artificial sequence
  Synthetic oligonucleotide
ccaccggtgaaagctctg

SEQ ID NO: 107 - DNA, 18 bp, Artificial sequence
  Synthetic oligonucleotide
tggacgggggaataatca

SEQ ID NO: 108 - DNA, 18 bp, Artificial sequence
  Synthetic oligonucleotide
gctcgtcctaccgtggag

SEQ ID NO: 109 - DNA, 19 bp, Artificial sequence
  Synthetic oligonucleotide
gcagataatgccacggtca

SEQ ID NO: 110 - DNA, 18 bp, Artificial sequence
  Synthetic oligonucleotide
tggtgttggagacggtga

SEQ ID NO: 111 - DNA, 18 bp, Artificial sequence
  Synthetic oligonucleotide
caaagggacgtccaatgc

SEQ ID NO: 112 - DNA, 18 bp, Artificial sequence
  Synthetic oligonucleotide
agcgaggtatgcggtgag

SEQ ID NO: 113 - DNA, 18 bp, Artificial sequence
  Synthetic oligonucleotide
agacggtcccggactacg

SEQ ID NO: 114 - DNA, 18 bp, Artificial sequence
  Synthetic oligonucleotide
gtggccctgtccatcaac

SEQ ID NO: 115 - DNA, 19 bp, Artificial sequence
  Synthetic oligonucleotide
ccggagcttcatgtaccag

SEQ ID NO: 116 - DNA, 19 bp, Artificial sequence
  Synthetic oligonucleotide
cttccaacaacggaactca

SEQ ID NO: 117 - DNA, 18 bp, Artificial sequence
  Synthetic oligonucleotide
cggactccgtgaccaacc

SEQ ID NO: 118 - DNA, 18 bp, Artificial sequence
  Synthetic oligonucleotide
ctgtgccccaggaacatc

SEQ ID NO: 119 - DNA, 19 bp, Artificial sequence
  Synthetic oligonucleotide
gcccattcgcctctaaagt

SEQ ID NO: 120 - DNA, 19 bp, Artificial sequence
  Synthetic oligonucleotide
ttccatttcattgcgggta

SEQ ID NO: 121 - DNA, 18 bp, Artificial sequence
  Synthetic oligonucleotide
ggcgacccaagttccttc

SEQ ID NO: 122 - DNA, 18 bp, Artificial sequence
  Synthetic oligonucleotide
cagacaccgtcctcacca

SEQ ID NO: 123 - DNA, 18 bp, Artificial sequence
  Synthetic oligonucleotide
gccctggagaggtcaggt

SEQ ID NO: 124 - DNA, 19 bp, Artificial sequence
  Synthetic oligonucleotide
acaccaccacgatgactcc

SEQ ID NO: 125 - DNA, 55 bp, Artificial sequence
  Synthetic oligonucleotide
cgtctacacccacttcccaagatgatgctacgccttcaatacctagtgtacagac

SEQ ID NO: 126 - DNA, 55 bp, Artificial sequence
  Synthetic oligonucleotide
gtacaatgacctagagattctcggaaactttgccaccttcagggagagagaggag

SEQ ID NO: 127 - DNA, 55 bp, Artificial sequence
  Synthetic oligonucleotide
gagaggtgaccagatctgtcctggaaatctcaaacctgatctattggagctctgg

SEQ ID NO: 128 - DNA, 55 bp, Artificial sequence
  Synthetic oligonucleotide
gtagcgtgtttgacctggagacgatgttcagggagtacaactactacacacatcg

SEQ ID NO: 129 - DNA, 55 bp, Artificial sequence
  Synthetic oligonucleotidev
gtggactgtagtgcgtcttagtcagcttattgagctcttcctgtatgtcccatcc

SEQ ID NO: 130 - DNA, 54 bp, Artificial sequence
  Synthetic oligonucleotide
ctgtggaaggatatcaactagagaggagggtccagccttattatggcaggagac

SEQ ID NO: 131 - DNA, 55 bp, Artificial sequence
  Synthetic oligonucleotide
gctatggtcgtcgaacatatgtataccgcctacacttatgtgtacacactcggcg

SEQ ID NO: 132 - DNA, 55 bp, Artificial sequence
  Synthetic oligonucleotide
ggtacgccatggtctgtagcatgtatctgcacgttatcgtctccatctattcgac

SEQ ID NO: 133 - DNA, 55 bp, Artificial sequence
  Synthetic oligonucleotide
cctgggtcctcttacgaatgtctgactacttcagccgcttgctgatatatgagtg

SEQ ID NO: 134 - DNA, 55 bp, Artificial sequence
  Synthetic oligonucleotide
caactacgggcgactatctaatcatcccatcgtatgacataccggcgatcatcac

SEQ ID NO: 135 - DNA, 55 bp, Artificial sequence
  Synthetic oligonucleotide
gactgctaggattcgtgcagccttgcataccctgtagatcgattgtgtatcctag

SEQ ID NO: 136 - DNA, 55 bp, Artificial sequence
  Synthetic oligonucleotide
cacaactcgaccacggagtctgacgtctacgtacttactgatcctcaagatactc

SEQ ID NO: 137 - DNA, 55 bp, Artificial sequence
  Synthetic oligonucleotide
gaacgtggcacagctctatctatagggaatgtgcgatctcggctatcgagatatg

SEQ ID NO: 138 - DNA, 55 bp, Artificial sequence
  Synthetic oligonucleotide
cagtcatagtctatgctcacctctgagtagcccggaatatagagggcgcttaaac

SEQ ID NO: 139 - DNA, 54 bp, Artificial sequence
  Synthetic oligonucleotide
gctccacggtctagtggcatacgcatccactatagacacctatataatccaggg

SEQ ID NO: 140 - DNA, 55 bp, Artificial sequence
  Synthetic oligonucleotide
gacggacagggtatctaactcctgaagtatctgatcccaggacgggtaatgatac

SEQ ID NO: 141 - DNA, 55 bp, Artificial sequence
  Synthetic oligonucleotide
cttcttcccacgtacatatatcctctccttgaaggttcgagagcgtaagagggag

SEQ ID NO: 142 - DNA, 55 bp, Artificial sequence
  Synthetic oligonucleotide
gtatcatacaacctcacggccgataggtagccacagttaagtgtgtcctcgtaag

SEQ ID NO: 143 - DNA, 55 bp, Artificial sequence
  Synthetic oligonucleotide
cacttctccgttacaggactggcttatagtcgcctatggtaacaaggaaggactg

SEQ ID NO: 144 - DNA, 55 bp, Artificial sequence
  Synthetic oligonucleotide
cactcctaggatacgggtcggtgcaggactacaaggagacggtacagataatatc

SEQ ID NO: 145 - DNA, 55 bp, Artificial sequence
  Synthetic oligonucleotide
catacagtacacccagtgtaagaatgtctgtggtgtgctgcgagaccctatagtg

SEQ ID NO: 146 - DNA, 55 bp, Artificial sequence
  Synthetic oligonucleotide
cctctgttcgccacgccaacttctcaaggagttctttctcctggtctataagttc

SEQ ID NO: 147 - DNA, 55 bp, Artificial sequence
  Synthetic oligonucleotide
cgtcctcctcatctgtctcctgctcctcctcatcatccttattgtcattgtcatc

SEQ ID NO: 148 - DNA, 54 bp, Artificial sequence
  Synthetic oligonucleotide
ggagcgatagatatactgctcctgggtatctgcctaaactcgctgtgtcttagc

SEQ ID NO: 149 - DNA, 55 bp, Artificial sequence
  Synthetic oligonucleotide
gggatcatccttctcagggagatgcattctttggaagtagtggtagagatggagc

SEQ ID NO: 150 - DNA, 55 bp, Artificial sequence
  Synthetic oligonucleotide
caatctgggcatcgacagagcatttggattacatggcgtgcacaacctgtcttac

SEQ ID NO: 151 - DNA, 55 bp, Artificial sequence
  Synthetic oligonucleotide
ggcagctagtctcattaaatcctattaacccgcagtgatcagtatcgttgatggc

SEQ ID NO: 152 - DNA, 55 bp, Artificial sequence
  Synthetic oligonucleotide
agccgaaaggattccaccattgtgctcgaatccaacggatttgacctcgtgttcc

SEQ ID NO: 153 - DNA, 55 bp, Artificial sequence
  Synthetic oligonucleotide
ctggtatacggaagcgggtgcgctcttcgtcttcccactctactccgggaaattt

SEQ ID NO: 154 - DNA, 52 bp, Artificial sequence
  Synthetic oligonucleotide
ctgtagaacggaaacatcgcatcccaatatgcttgccagctgaggaactacc

SEQ ID NO: 155 - DNA, 54 bp, Artificial sequence
  Synthetic oligonucleotide
ttcagtgttgtgtgcgtcagtctagtgaggtacctcctggtggcatattctacg

SEQ ID NO: 156 - DNA, 55 bp, Artificial sequence
  Synthetic oligonucleotide
gtctgtacactaggtattgaaggcgtagcatcatcttgggaagtgggtgtagacg

SEQ ID NO: 157 - DNA, 55 bp, Artificial sequence
  Synthetic oligonucleotide
ctcctctctctccctgaaggtggcaaagtttccgagaatctctaggtcattgtac

SEQ ID NO: 158 - DNA, 55 bp, Artificial sequence
  Synthetic oligonucleotide
ccagagctccaatagatcaggtttgagatttccaggacagatctggtcacctctc

SEQ ID NO: 159 - DNA, 55 bp, Artificial sequence
  Synthetic oligonucleotide
cgatgtgtgtagtagttgtactccctgaacatcgtctccaggtcaaacacgctac

SEQ ID NO: 160 - DNA, 55 bp, Artificial sequence
  Synthetic oligonucleotide
ggatgggacatacaggaagagctcaataagctgactaagacgcactacagtccac

SEQ ID NO: 161 - DNA, 54 bp, Artificial sequence
  Synthetic oligonucleotide
gtctcctgccataataaggctggaccctcctctctagttgatatccttccacag

SEQ ID NO: 162 - DNA, 55 bp, Artificial sequence
  Synthetic oligonucleotide
cgccgagtgtgtacacataagtgtaggcggtatacatatgttcgacgaccatagc

SEQ ID NO: 163 - DNA, 55 bp, Artificial sequence
  Synthetic oligonucleotide
gtcgaatagatggagacgataacgtgcagatacatgctacagaccatggcgtacc

SEQ ID NO: 164 - DNA, 55 bp, Artificial sequence
  Synthetic oligonucleotide
cactcatatatcagcaagcggctgaagtagtcagacattcgtaagaggacccagg

SEQ ID NO: 165 - DNA, 55 bp, Artificial sequence
  Synthetic oligonucleotide
gtgatgatcgccggtatgtcatacgatgggatgattagatagtcgcccgtagttg

SEQ ID NO: 166 - DNA, 55 bp, Artificial sequence
  Synthetic oligonucleotide
ctaggatacacaatcgatctacagggtatgcaaggctgcacgaatcctagcagtc

SEQ ID NO: 167 - DNA, 55 bp, Artificial sequence
  Synthetic oligonucleotide
gagtatcttgaggatcagtaagtacgtagacgtcagactccgtggtcgagttgtg

SEQ ID NO: 168 - DNA, 55 bp, Artificial sequence
  Synthetic oligonucleotide
catatctcgatagccgagatcgcacattccctatagatagagctgtgccacgttc

SEQ ID NO: 169 - DNA, 55 bp, Artificial sequence
  Synthetic oligonucleotide
gtttaagcgccctctatattccgggctactcagaggtgagcatagactatgactg

SEQ ID NO: 170 - DNA, 54 bp, Artificial sequence
  Synthetic oligonucleotide
ccctggattatataggtgtctatagtggatgcgtatgccactagaccgtggagc

SEQ ID NO: 171 - DNA, 55 bp, Artificial sequence
  Synthetic oligonucleotide
gtatcattacccgtcctgggatcagatacttcaggagttagataccctgtccgtc

SEQ ID NO: 172 - DNA, 55 bp, Artificial sequence
  Synthetic oligonucleotide
ctccctcttacgctctcgaaccttcaaggagaggatatatgtacgtgggaagaag

SEQ ID NO: 173 - DNA, 55 bp, Artificial sequence
  Synthetic oligonucleotide
cttacgaggacacacttaactgtggctacctatcggccgtgaggttgtatgatac

SEQ ID NO: 174 - DNA, 55 bp, Artificial sequence
  Synthetic oligonucleotide
cagtccttccttgttaccataggcgactataagccagtcctgtaacggagaagtg

SEQ ID NO: 175 - DNA, 55 bp, Artificial sequence
  Synthetic oligonucleotide
gatattatctgtaccgtctccttgtagtcctgcaccgacccgtatcctaggagtg

SEQ ID NO: 176 - DNA, 55 bp, Artificial sequence
  Synthetic oligonucleotide
cactatagggtctcgcagcacaccacagacattcttacactgggtgtactgtatg

SEQ ID NO: 177 - DNA, 55 bp, Artificial sequence
  Synthetic oligonucleotide
gaacttatagaccaggagaaagaactccttgagaagttggcgtggcgaacagagg

SEQ ID NO: 178 - DNA, 55 bp, Artificial sequence
  Synthetic oligonucleotide
gatgacaatgacaataaggatgatgaggaggagcaggagacagatgaggaggacg

SEQ ID NO: 179 - DNA, 54 bp, Artificial sequence
  Synthetic oligonucleotide
gctaagacacagcgagtttaggcagatacccaggagcagtatatctatcgctcc

SEQ ID NO: 180 - DNA, 55 bp, Artificial sequence
  Synthetic oligonucleotide
gctccatctctaccactacttccaaagaatgcatctccctgagaaggatgatccc

SEQ ID NO: 181 - DNA, 55 bp, Artificial sequence
  Synthetic oligonucleotide
gtaagacaggttgtgcacgccatgtaatccaaatgctctgtcgatgcccagattg

SEQ ID NO: 182 - DNA, 55 bp, Artificial sequence
  Synthetic oligonucleotide
gccatcaacgatactgatcactgcgggttaataggatttaatgagactagctgcc

SEQ ID NO: 183 - DNA, 55 bp, Artificial sequence
  Synthetic oligonucleotide
ggaacacgaggtcaaatccgttggattcgagcacaatggtggaatcctttcggct

SEQ ID NO: 184 - DNA, 55 bp, Artificial sequence
  Synthetic oligonucleotide
aaatttcccggagtagagtgggaagacgaagagcgcacccgcttccgtataccag

SEQ ID NO: 185 - DNA, 52 bp, Artificial sequence
  Synthetic oligonucleotide
ggtagttcctcagctggcaagcatattgggatgcgatgtttccgttctacag

SEQ ID NO: 186 - DNA, 54 bp, Artificial sequence
  Synthetic oligonucleotide
cgtagaatatgccaccaggaggtacctcactagactgacgcacacaacactgaa

SEQ ID NO: 187 - DNA, 55 bp, Artificial sequence
  Synthetic oligonucleotide
gatggagaggcaaacatacaggaggaaaggctatatgagctactctctgacccac

SEQ ID NO: 188 - DNA, 55 bp, Artificial sequence
  Synthetic oligonucleotide
gatagtcttggaaacccgtcactctcagtaattccctcgaatccctaccaggaac

SEQ ID NO: 189 - DNA, 55 bp, Artificial sequence
  Synthetic oligonucleotide
gttacctggtatttctgacatcccagttctgctacgaagagtacgtgcagaggac

SEQ ID NO: 190 - DNA, 55 bp, Artificial sequence
  Synthetic oligonucleotide
ccctgacctgtcccagggtcttcaggttaaacagatattgagaggagacaaagag

SEQ ID NO: 191 - DNA, 55 bp, Artificial sequence
  Synthetic oligonucleotide
cttaaggccgagtcagttacacacacagtagccgaatatctggaggtcttctctg

SEQ ID NO: 192 - DNA, 55 bp, Artificial sequence
  Synthetic oligonucleotide
ctcatctcaagggaggagtgctgcaggtaaaccttctgtctgtaaactatggagg

SEQ ID NO: 193 - DNA, 55 bp, Artificial sequence
  Synthetic oligonucleotide
ctgactcatgaaggtgaccgtgatggcctgtgatgtgtagtagagtaccagaaac

SEQ ID NO: 194 - DNA, 55 bp, Artificial sequence
  Synthetic oligonucleotide
ctccatctgggcacttctgacgcttgtcttagtcattatagcctcagccatctac

SEQ ID NO: 195 - DNA, 55 bp, Artificial sequence
  Synthetic oligonucleotide
gaatctgtcagtgaccactatcaggtggtctaacacgtagcgcatcactataggg

SEQ ID NO: 196 - DNA, 55 bp, Artificial sequence
  Synthetic oligonucleotide
ctataactacattcagggatctatagccaccatctcccagcttctgcacctcgag

SEQ ID NO: 197 - DNA, 55 bp, Artificial sequence
  Synthetic oligonucleotide
cattgcagaaggtttaagagctctcctggctaggagtcacgtagaaaggactacc

SEQ ID NO: 198 - DNA, 55 bp, Artificial sequence
  Synthetic oligonucleotide
gaggacgtgagtgacactgatgagtctgactactcagatgaagacgaggagattg

SEQ ID NO: 199 - DNA, 54 bp, Artificial sequence
  Synthetic oligonucleotide
gcagcgtctctacgtcagatactcgtcagacacgatctctatattattgggccc

SEQ ID NO: 200 - DNA, 55 bp, Artificial sequence
  Synthetic oligonucleotide
cctcccttcttcggccgctattagcttagtagtctccaggttaaactcctcatag

SEQ ID NO: 201 - DNA, 55 bp, Artificial sequence
  Synthetic oligonucleotide
cttacggacatctttaagattccaggcctcatcctgcgtcaacagatagtcaccc

SEQ ID NO: 202 - DNA, 55 bp, Artificial sequence
  Synthetic oligonucleotide
gaatcatgtcacacaccatgagctcgtgatacagctccgtcacagagtcatagag

SEQ ID NO: 203 - DNA, 54 bp, Artificial sequence
  Synthetic oligonucleotide
ccatgtacctcctgactaatgagaagtccaaggcctttgagaggctcatctacg

SEQ ID NO: 204 - DNA, 55 bp, Artificial sequence
  Synthetic oligonucleotide
caaacaccagtgtccagagaggaagaccgtaagataaagatggctgcctctcatc

SEQ ID NO: 205 - DNA, 55 bp, Artificial sequence
  Synthetic oligonucleotide
ggctcagctagggtctctgcctctccatcatagacatcttccttgaatctcattc

SEQ ID NO: 206 - DNA, 55 bp, Artificial sequence
  Synthetic oligonucleotide
cgtaggtctgacctggaacaatcttggtgagtatcaaactgtccacgctaacctc

SEQ ID NO: 207 - DNA, 55 bp, Artificial sequence
  Synthetic oligonucleotide
ctcatctcccttctcggtcactcgcttgtaggtgcccatcagaaatttagaagtc

SEQ ID NO: 208 - DNA, 55 bp, Artificial sequence
  Synthetic oligonucleotide
gagaagagggcctgcggaaattagactcatcctcagactcacagtcagatttgtc

SEQ ID NO: 209 - DNA, 55 bp, Artificial sequence
  Synthetic oligonucleotide
gggattatcagagagacggaggtgttggagtcatttacccattctagggtaaggc

SEQ ID NO: 210 - DNA, 55 bp, Artificial sequence
  Synthetic oligonucleotide
gtagatggtatccatctggtcagtttcgtagctgtcaacggagaacttctcctcg

SEQ ID NO: 211 - DNA, 55 bp, Artificial sequence
  Synthetic oligonucleotide
cgttgatgatgtagttctccctcctggtagtggacttgatgaagctgttctggag

SEQ ID NO: 212 - DNA, 47 bp, Artificial sequence
  Synthetic oligonucleotide
gatttggacccgaaatctgacactttagagctctggaggactttaaa

SEQ ID NO: 213 - DNA, 55 bp, Artificial sequence
  Synthetic oligonucleotide
ggccgtttggcgtctcaggctatgaagaagattgaagacaaggttcggaaatctg

SEQ ID NO: 214 - DNA, 55 bp, Artificial sequence; identical to SEQ ID NO: 197
  Synthetic oligonucleotide

SEQ ID NO: 215 - DNA, 52 bp, Artificial sequence
  Synthetic oligonucleotide
tttgctagggaggagacgtgtgtggctgtagccacccgtcccgggtacaagt

SEQ ID NO: 216 - DNA, 54 bp, Artificial sequence
  Synthetic oligonucleotide
gtagaagggtcctcgtccagcaagaagaggaggtggtaagcggttcaccttcag

SEQ ID NO: 217 - DNA, 55 bp, Artificial sequence
  Synthetic oligonucleotide
ctcgttggagttagagtcagattcatggccagaatcatcggtagcttgttgaggg

SEQ ID NO: 218 - DNA, 55 bp, Artificial sequence
  Synthetic oligonucleotide
gtgggtcagagagtagctcatatagcctttcctcctgtatgtttgcctctccatc

SEQ ID NO: 219 - DNA, 55 bp, Artificial sequence
  Synthetic oligonucleotide
gttcctggtagggattcgagggaattactgagagtgacgggtttccaagactatc

SEQ ID NO: 220 - DNA, 55 bp, Artificial sequence
  Synthetic oligonucleotide
gtcctctgcacgtactcttcgtagcagaactgggatgtcagaaataccaggtaac

SEQ ID NO: 221 - DNA, 55 bp, Artificial sequence
  Synthetic oligonucleotide
ctctttgtctcctctcaatatctgtttaacctgaagaccctgggacaggtcaggg

SEQ ID NO: 222 - DNA, 55 bp, Artificial sequence
  Synthetic oligonucleotide
cagagaagacctccagatattcggctactgtgtgtgtaactgactcggccttaag

SEQ ID NO: 223 - DNA, 55 bp, Artificial sequence
  Synthetic oligonucleotide
cctccatagtttacagacagaaggtttacctgcagcactcctcccttgagatgag

SEQ ID NO: 224 - DNA, 55 bp, Artificial sequence
  Synthetic oligonucleotide
gtttctggtactctactacacatcacaggccatcacggtcaccttcatgagtcag

SEQ ID NO: 225 - DNA, 55 bp, Artificial sequence
  Synthetic oligonucleotide
gtagatggctgaggctataatgactaagacaagcgtcagaagtgcccagatggag

SEQ ID NO: 226 - DNA, 55 bp, Artificial sequence
  Synthetic oligonucleotide
ccctatagtgatgcgctacgtgttagaccacctgatagtggtcactgacagattc

SEQ ID NO: 227 - DNA, 55 bp, Artificial sequence
  Synthetic oligonucleotide
ctcgaggtgcagaagctgggagatggtggctatagatccctgaatgtagttatag

SEQ ID NO: 228 - DNA, 55 bp, Artificial sequence
  Synthetic oligonucleotide
ggtagtcctttctacgtgactcctagccaggagagctcttaaaccttctgcaatg

SEQ ID NO: 229 - DNA, 55 bp, Artificial sequence
  Synthetic oligonucleotide
caatctcctcgtcttcatctgagtagtcagactcatcagtgtcactcacgtcctc

SEQ ID NO: 230 - DNA, 54 bp, Artificial sequence
  Synthetic oligonucleotide
gggcccaataatatagagatcgtgtctgacgagtatctgacgtagagacgctgc

SEQ ID NO: 231 - DNA, 55 bp, Artificial sequence
  Synthetic oligonucleotide
ctatgaggagtttaacctggagactactaagctaatagcggccgaagaagggagg

SEQ ID NO: 232 - DNA, 55 bp, Artificial sequence
  Synthetic oligonucleotide
gggtgactatctgttgacgcaggatgaggcctggaatcttaaagatgtccgtaag

SEQ ID NO: 233 - DNA, 55 bp, Artificial sequence
  Synthetic oligonucleotide
ctctatgactctgtgacggagctgtatcacgagctcatggtgtgtgacatgattc

SEQ ID NO: 234 - DNA, 54 bp, Artificial sequence
  Synthetic oligonucleotide
cgtagatgagcctctcaaaggccttggacttctcattagtcaggaggtacatgg

SEQ ID NO: 235 - DNA, 55 bp, Artificial sequence
  Synthetic oligonucleotide
gatgagaggcagccatctttatcttacggtcttcctctctggacactggtgtttg

SEQ ID NO: 236 - DNA, 55 bp, Artificial sequence
  Synthetic oligonucleotide
gaatgagattcaaggaagatgtctatgatggagaggcagagaccctagctgagcc

SEQ ID NO: 237 - DNA, 55 bp, Artificial sequence
  Synthetic oligonucleotide
gaggttagcgtggacagtttgatactcaccaagattgttccaggtcagacctacg

SEQ ID NO: 238 - DNA, 55 bp, Artificial sequence
  Synthetic oligonucleotide
gacttctaaatttctgatgggcacctacaagcgagtgaccgagaagggagatgag

SEQ ID NO: 239 - DNA, 55 bp, Artificial sequence
  Synthetic oligonucleotide
gacaaatctgactgtgagtctgaggatgagtctaatttccgcaggccctcttctc

SEQ ID NO: 240 - DNA, 55 bp, Artificial sequence
  Synthetic oligonucleotide
gccttaccctagaatgggtaaatgactccaacacctccgtctctctgataatccc

SEQ ID NO: 241 - DNA, 55 bp, Artificial sequence
  Synthetic oligonucleotide
cgaggagaagttctccgttgacagctacgaaactgaccagatggataccatctac

SEQ ID NO: 242 - DNA, 55 bp, Artificial sequence
  Synthetic oligonucleotide
ctccagaacagcttcatcaagtccactaccaggagggagaactacatcatcaacg

SEQ ID NO: 243 - DNA, 47 bp, Artificial sequence
  Synthetic oligonucleotide
tttaaagtcctccagagctctaaagtgtcagatttcgggtccaaatc

SEQ ID NO: 244 - DNA, 55 bp, Artificial sequence
  Synthetic oligonucleotide
cagatttccgaaccttgtcttcaatcttcttcatagcctgagacgccaaacggcc

SEQ ID NO: 245 - DNA, 55 bp, Artificial sequence; identical to SEQ ID NO: 228
  Synthetic oligonucleotide

SEQ ID NO: 246 - DNA, 52 bp, Artificial sequence
  Synthetic oligonucleotide
acttgtacccgggacgggtggctacagccacacacgtctcctccctagcaaa

SEQ ID NO: 247 - DNA, 54 bp, Artificial sequence
  Synthetic oligonucleotide
ctgaaggtgaaccgcttaccacctcctcttcttgctggacgaggacccttctac

SEQ ID NO: 248 - DNA, 55 bp, Artificial sequence
  Synthetic oligonucleotide
ccctcaacaagctaccgatgattctggccatgaatctgactctaactccaacgag

SEQ ID NO: 249 - DNA, 24 bp, Artificial sequence
  Synthetic oligonucleotide
agacaagacgtcgggaccaattac

SEQ ID NO: 250 - DNA, 20 bp, Artificial sequence
  Synthetic oligonucleotide
atgaggatgccgagtatgga

SEQ ID NO: 251 - DNA, 24 bp, Artificial sequence
  Synthetic oligonucleotide
ccgctaatggaaacgcagtgaatg

SEQ ID NO: 252 - DNA, 24 bp, Artificial sequence
  Synthetic oligonucleotide
gcgttgaacgagtgcaagtagaac

SEQ ID NO: 253 - DNA, 24 bp, Artificial sequence
  Synthetic oligonucleotide
ttgcgagaagggatcgttggatac

SEQ ID NO: 254 - DNA, 22 bp, Artificial sequence
  Synthetic oligonucleotide
taaatcaaccgccaatgatgcg

SEQ ID NO: 255 - DNA, 24 bp, Artificial sequence
  Synthetic oligonucleotide
tatttgaaatcgcgactccgggac

SEQ ID NO: 256 - DNA, 22 bp, Artificial sequence
  Synthetic oligonucleotide
aaatcaaccgccaatgatgcgg

SEQ ID NO: 257 - DNA, 23 bp, Artificial sequence
  Synthetic oligonucleotide
ttgagcgggtcatctggtttagg

SEQ ID NO: 258 - DNA, 23 bp, Artificial sequence
  Synthetic oligonucleotide
agattgctctttcagtggctggt

SEQ ID NO: 259 - DNA, 22 bp, Artificial sequence
  Synthetic oligonucleotide
atcgcgactccggaaccaatta

SEQ ID NO: 260 - DNA, 22 bp, Artificial sequence
  Synthetic oligonucleotide
aaatcgcgactccggaaccaat

SEQ ID NO: 261 - DNA, 24 bp, Artificial sequence
  Synthetic oligonucleotide
gacgagactccggaaccaattact

SEQ ID NO: 262 - DNA, 24 bp, Artificial sequence
  Synthetic oligonucleotide
gccattcttcccatggatgtttcc

SEQ ID NO: 263 - DNA, 22 bp, Artificial sequence
  Synthetic oligonucleotide
caaacgcggtgacatgtgtgat

SEQ ID NO: 264 - DNA, 22 bp, Artificial sequence
  Synthetic oligonucleotide
ggcatcaagacgtggcaaacaa

SEQ ID NO: 265 - DNA, 26 bp, Artificial sequence
  Synthetic oligonucleotide
ggaagagtattgtacgggactatgcg

SEQ ID NO: 266 - DNA, 24 bp, Artificial sequence
  Synthetic oligonucleotide
atctccagttgaaccgaacacctc

SEQ ID NO: 267 - DNA, 26 bp, Artificial sequence
  Synthetic oligonucleotide
agacgatgtgagaagactgaagagga

SEQ ID NO: 268 - DNA, 23 bp, Artificial sequence
  Synthetic oligonucleotide
cattgactgctagagatgccggt

SEQ ID NO: 269 - DNA, 22 bp, Artificial sequence
  Synthetic oligonucleotide
actatcggcaattgcactcgga

SEQ ID NO: 270 - DNA, 22 bp, Artificial sequence
  Synthetic oligonucleotide
acagggtttgtgctgagatggt

SEQ ID NO: 271 - DNA, 22 bp, Artificial sequence
  Synthetic oligonucleotide
caggcagtgtgtcatcagcatt

SEQ ID NO: 272 - DNA, 22 bp, Artificial sequence
  Synthetic oligonucleotide
cgctggcaacaacaacactcat

SEQ ID NO: 273 - DNA, 22 bp, Artificial sequence
  Synthetic oligonucleotide
tgccttccataaagagggtgct

SEQ ID NO: 274 - DNA, 24 bp, Artificial sequence
  Synthetic oligonucleotide
actctatgtgctgtgatgacgagg

SEQ ID NO: 275 - DNA, 22 bp, Artificial sequence
  Synthetic oligonucleotide
tgtgggcattgagagtcatcct

SEQ ID NO: 276 - DNA, 24 bp, Artificial sequence
  Synthetic oligonucleotide
tcttcaagggaccctggctagtat

SEQ ID NO: 277 - DNA, 24 bp, Artificial sequence
  Synthetic oligonucleotide
gttcgagcacgatcatcatccaga

SEQ ID NO: 278 - DNA, 22 bp, Artificial sequence
  Synthetic oligonucleotide
agtcaaagagagtgccagagca

SEQ ID NO: 279 - DNA, 20 bp, Artificial sequence
  Synthetic oligonucleotide
gccttatccgactcgcaatg

SEQ ID NO: 280 - DNA, 22 bp, Artificial sequence
  Synthetic oligonucleotide
tgccgtatgactgcaaggaact

SEQ ID NO: 281 - DNA, 24 bp, Artificial sequence
  Synthetic oligonucleotide
ggccctggaatcgaaggactttat

SEQ ID NO: 282 - DNA, 24 bp, Artificial sequence
  Synthetic oligonucleotide
ggactacaatgcagcccttgtcta

SEQ ID NO: 283 - DNA, 24 bp, Artificial sequence
  Synthetic oligonucleotide
ctgcctctcgggattatgagcaat

SEQ ID NO: 284 - DNA, 22 bp, Artificial sequence
  Synthetic oligonucleotide
gcaaccgattaagcgccgtaaa

SEQ ID NO: 285 - DNA, 22 bp, Artificial sequence
  Synthetic oligonucleotide
ttgcggcaacgctaattacagg

SEQ ID NO: 286 - DNA, 22 bp, Artificial sequence
  Synthetic oligonucleotide
gtagcaggagctattgcaacga

SEQ ID NO: 287 - DNA, 22 bp, Artificial sequence
  Synthetic oligonucleotide
tgactatgtgggtgctggtgaa

SEQ ID NO: 288 - DNA, 22 bp, Artificial sequence
  Synthetic oligonucleotide
gcagcaactacagcagcatcaa

SEQ ID NO: 289 - DNA, 22 bp, Artificial sequence
  Synthetic oligonucleotide
ggaaatgcagaagtgcatgcgt

SEQ ID NO: 290 - DNA, 22 bp, Artificial sequence
  Synthetic oligonucleotide
agcaaccctaggtgcggattta

SEQ ID NO: 291 - DNA, 24 bp, Artificial sequence
  Synthetic oligonucleotide
cagcaattactttgagtggtcccg

SEQ ID NO: 292 - DNA, 22 bp, Artificial sequence
  Synthetic oligonucleotide
ttgcacctgaccatagaacggt

SEQ ID NO: 293 - DNA, 23 bp, Artificial sequence
  Synthetic oligonucleotide
accctaggtgcggatttagttga

SEQ ID NO: 294 - DNA, 22 bp, Artificial sequence
  Synthetic oligonucleotide
cgcgaaatggttatggctctac

SEQ ID NO: 295 - DNA, 22 bp, Artificial sequence
  Synthetic oligonucleotide
agctactaatgcgtcacaggca

SEQ ID NO: 296 - DNA, 25 bp, Artificial sequence
  Synthetic oligonucleotide
agacaggttcttaactgaaagttct

SEQ ID NO: 297 - DNA, 24 bp, Artificial sequence
  Synthetic oligonucleotide
tcggctatcataagaggtgcttgc

SEQ ID NO: 298 - DNA, 24 bp, Artificial sequence
  Synthetic oligonucleotide
tctagggttaggtggctctgatga

SEQ ID NO: 299 - DNA, 24 bp, Artificial sequence
  Synthetic oligonucleotide
ggaattactgggcgtaaagggtct

SEQ ID NO: 300 - DNA, 23 bp, Artificial sequence
  Synthetic oligonucleotide
acgcaagctactgctaagcaaac

SEQ ID NO: 301 - DNA, 24 bp, Artificial sequence
  Synthetic oligonucleotide
ggatatcgtccattccgacagcat

SEQ ID NO: 302 - DNA, 23 bp, Artificial sequence
  Synthetic oligonucleotide
ttagcataccctgcttgttcgcc

SEQ ID NO: 303 - DNA, 22 bp, Artificial sequence
  Synthetic oligonucleotide
agtatgaggcaatgagctgcga

SEQ ID NO: 304 - DNA, 22 bp, Artificial sequence
  Synthetic oligonucleotide
tttgcaacgtgtggccttgttg

SEQ ID NO: 305 - DNA, 24 bp, Artificial sequence
  Synthetic oligonucleotide
ccgaaactaaatagcgctggttcc

SEQ ID NO: 306 - DNA, 24 bp, Artificial sequence
  Synthetic oligonucleotide
caggaaagggaaactgggactgta

SEQ ID NO: 307 - DNA, 24 bp, Artificial sequence
  Synthetic oligonucleotide
tatgaccatgcctctctcttgcac

SEQ ID NO: 308 - DNA, 24 bp, Artificial sequence
  Synthetic oligonucleotide
agcaggactccaagtattcacacg

SEQ ID NO: 309 - DNA, 24 bp, Artificial sequence
  Synthetic oligonucleotide
tggtcttaagctgtcaaggctctg

SEQ ID NO: 310 - DNA, 24 bp, Artificial sequence
  Synthetic oligonucleotide
acctcagtatcagagggaactcca

SEQ ID NO: 311 - DNA, 24 bp, Artificial sequence
  Synthetic oligonucleotide
gtacaacgtcattgagcggagtct

SEQ ID NO: 312 - DNA, 22 bp, Artificial sequence
  Synthetic oligonucleotide
aagtgctgggctcatagttggt

SEQ ID NO: 313 - DNA, 24 bp, Artificial sequence
  Synthetic oligonucleotide
ccagtgatgaccaggttagcctta

SEQ ID NO: 314 - DNA, 26 bp, Artificial sequence
  Synthetic oligonucleotide
cctctagtgacgatcagatcactctt

SEQ ID NO: 315 - DNA, 24 bp, Artificial sequence
  Synthetic oligonucleotide
tctatgtgggaggtgagagactgt

SEQ ID NO: 316 - DNA, 22 bp, Artificial sequence
  Synthetic oligonucleotide
tattgaaggcccgccaactgat

SEQ ID NO: 317 - DNA, 24 bp, Artificial sequence
  Synthetic oligonucleotide
gggaacagtccagaatctttgagc

SEQ ID NO: 318 - DNA, 24 bp, Artificial sequence
  Synthetic oligonucleotide
tctgttcgtccagtccaaccatac

SEQ ID NO: 319 - DNA, 24 bp, Artificial sequence
  Synthetic oligonucleotide
aacaaggaggctaactccacgaag

SEQ ID NO: 320 - DNA, 22 bp, Artificial sequence
  Synthetic oligonucleotide
tgagatgggtgtctgctttgga

SEQ ID NO: 321 - DNA, 22 bp, Artificial sequence
  Synthetic oligonucleotide
acctcaatcaacaagcccaggt

SEQ ID NO: 322 - DNA, 22 bp, Artificial sequence
  Synthetic oligonucleotide
attggtacgggctgtactgcat

SEQ ID NO: 323 - DNA, 22 bp, Artificial sequence
  Synthetic oligonucleotide
agccatctgctatggtgcagaa

SEQ ID NO: 324 - DNA, 24 bp, Artificial sequence
  Synthetic oligonucleotide
ggctgcaagtgcatcagagaatgt

SEQ ID NO: 325 - DNA, 22 bp, Artificial sequence
  Synthetic oligonucleotide
atgggtcagggattgtgcagat

SEQ ID NO: 326 - DNA, 22 bp, Artificial sequence
  Synthetic oligonucleotide
actcagcactgcacatgaggtt

SEQ ID NO: 327 - DNA, 24 bp, Artificial sequence
  Synthetic oligonucleotide
tgtgggtagggctagagtatcaca

SEQ ID NO: 328 - DNA, 24 bp, Artificial sequence
  Synthetic oligonucleotide
tcctggtggtaaggactagaggtt

SEQ ID NO: 329 - DNA, 24 bp, Artificial sequence
  Synthetic oligonucleotide
aagaggagatctacctgtctggct

SEQ ID NO: 330 - DNA, 24 bp, Artificial sequence
  Synthetic oligonucleotide
ttaccaaattccctggaggagctg

SEQ ID NO: 331 - DNA, 20 bp, Artificial sequence
  Synthetic oligonucleotide
tcctgcgcaaactgtgcatt

SEQ ID NO: 332 - DNA, 24 bp, Artificial sequence
  Synthetic oligonucleotide
gtgcgttggaaatcgaagagatgc

SEQ ID NO: 333 - DNA, 24 bp, Artificial sequence
  Synthetic oligonucleotide
ctgtacaacgcacgtatcatccct

SEQ ID NO: 334 - DNA, 24 bp, Artificial sequence
  Synthetic oligonucleotide
tgtacttccacagaagagacctgc

SEQ ID NO: 335 - DNA, 24 bp, Artificial sequence
  Synthetic oligonucleotide
ccatttcttccgtagcttccctga

SEQ ID NO: 336 - DNA, 24 bp, Artificial sequence
  Synthetic oligonucleotide
cttcgccacatcactacacttcct

SEQ ID NO: 337 - DNA, 24 bp, Artificial sequence
  Synthetic oligonucleotide
accaccttatgtccttcccacaag

SEQ ID NO: 338 - DNA, 24 bp, Artificial sequence
  Synthetic oligonucleotide
gtagcagaggctgttgtgaagact

SEQ ID NO: 339 - DNA, 22 bp, Artificial sequence
  Synthetic oligonucleotide
attaattgggtgactggcggga

SEQ ID NO: 340 - DNA, 22 bp, Artificial sequence
  Synthetic oligonucleotide
acttccctacggcgctaacaaa

SEQ ID NO: 341 - DNA, 26 bp, Artificial sequence
  Synthetic oligonucleotide
cattcgtttcggaagaaacaggtacg

SEQ ID NO: 342 - DNA, 25 bp, Artificial sequence
  Synthetic oligonucleotide
cttgttaaagacccaccgaatgtgc

SEQ ID NO: 343 - DNA, 24 bp, Artificial sequence
  Synthetic oligonucleotide
cacctacacacctcagcgttgata

SEQ ID NO: 344 - DNA, 22 bp, Artificial sequence
  Synthetic oligonucleotide
agctaacgagtgtgcgcaagta

SEQ ID NO: 345 - DNA, 24 bp, Artificial sequence
  Synthetic oligonucleotide
gttggcgacacagtagatggttca

SEQ ID NO: 346 - DNA, 24 bp, Artificial sequence
  Synthetic oligonucleotide
gcacatatgctctcgtatccgact

SEQ ID NO: 347 - DNA, 24 bp, Artificial sequence
  Synthetic oligonucleotide
gtcgctgtctttctcttcttcgct

SEQ ID NO: 348 - DNA, 24 bp, Artificial sequence
  Synthetic oligonucleotide
gcgatatacgcgactgttcccttt

SEQ ID NO: 349 - DNA, 21 bp, Artificial sequence
  Synthetic oligonucleotide
gaagctcttcgccgcgacttt

SEQ ID NO: 350 - DNA, 24 bp, Artificial sequence
  Synthetic oligonucleotide
ttggcgacacagtagatggttcat

SEQ ID NO: 351 - DNA, 22 bp, Artificial sequence
  Synthetic oligonucleotide
aattggtcccgacgtcttgtct

SEQ ID NO: 352 - DNA, 19 bp, Artificial sequence
  Synthetic oligonucleotide
aaattgccacggccgacaa

SEQ ID NO: 353 - DNA, 23 bp, Artificial sequence
  Synthetic oligonucleotide
cccttccagattgcctctctgtt

SEQ ID NO: 354 - DNA, 26 bp, Artificial sequence
  Synthetic oligonucleotide
aacgtagtagctatagccgcgtctcc

SEQ ID NO: 355 - DNA, 22 bp, Artificial sequence
  Synthetic oligonucleotide
aattggttccggagtctcgtct

SEQ ID NO: 356 - DNA, 22 bp, Artificial sequence
  Synthetic oligonucleotide
aattggttccggagtctcgtct

SEQ ID NO: 357 - DNA, 24 bp, Artificial sequence
  Synthetic oligonucleotide
gatccgcatcatcggtggttgatt

SEQ ID NO: 358 - DNA, 24 bp, Artificial sequence
  Synthetic oligonucleotide
aactcggaagctcgtcatgtagac

SEQ ID NO: 359 - DNA, 22 bp, Artificial sequence
  Synthetic oligonucleotide
ttcgagaaacccttctgtggct

SEQ ID NO: 360 - DNA, 22 bp, Artificial sequence
  Synthetic oligonucleotide
acggatggtcgtcgtattcagt

SEQ ID NO: 361 - DNA, 22 bp, Artificial sequence
  Synthetic oligonucleotide
atcacacatgtcaccgcgtttg

SEQ ID NO: 362 - DNA, 24 bp, Artificial sequence
  Synthetic oligonucleotide
atctccagttgaaccgaacacctc

SEQ ID NO: 363 - DNA, 26 bp, Artificial sequence
  Synthetic oligonucleotide
tcggtgtttcgtatatccctgaatcc

SEQ ID NO: 364 - DNA, 25 bp, Artificial sequence
  Synthetic oligonucleotide
tcagtgtcatttgtaggcgatgtca

SEQ ID NO: 365 - DNA, 22 bp, Artificial sequence
  Synthetic oligonucleotide
tcaagttcgcgagactctgcat

SEQ ID NO: 366 - DNA, 22 bp, Artificial sequence
  Synthetic oligonucleotide
aacactttgagggacggtctca

SEQ ID NO: 367 - DNA, 22 bp, Artificial sequence
  Synthetic oligonucleotide
tatgaagcaagcatgatggcgg

SEQ ID NO: 368 - DNA, 22 bp, Artificial sequence
  Synthetic oligonucleotide
gggttgcatttgggttgagcat

SEQ ID NO: 369 - DNA, 24 bp, Artificial sequence
  Synthetic oligonucleotide
cagaaatgcaacgacaccttcagc

SEQ ID NO: 370 - DNA, 22 bp, Artificial sequence
  Synthetic oligonucleotide
tcggtcccattgttgccatagt

SEQ ID NO: 371 - DNA, 22 bp, Artificial sequence
  Synthetic oligonucleotide
ccttgacacgttgtgttcgcat

SEQ ID NO: 372 - DNA, 22 bp, Artificial sequence
  Synthetic oligonucleotide
aatccagaggtttgccgagtgt

SEQ ID NO: 373 - DNA, 24 bp, Artificial sequence
  Synthetic oligonucleotide
gtctttaggtgctggaggaactgt

SEQ ID NO: 374 - DNA, 22 bp, Artificial sequence
  Synthetic oligonucleotide
tagcaaggcttctgcgagtgtt

SEQ ID NO: 375 - DNA, 22 bp, Artificial sequence
  Synthetic oligonucleotide
cacttgtgtggtgccatgatgc

SEQ ID NO: 376 - DNA, 24 bp, Artificial sequence
  Synthetic oligonucleotide
gccagacgattaacagagggatgt

SEQ ID NO: 377 - DNA, 24 bp, Artificial sequence
  Synthetic oligonucleotide
gtcctttcctcggttgtgactctt

SEQ ID NO: 378 - DNA, 24 bp, Artificial sequence
  Synthetic oligonucleotide
gccgctcaatttcatggactcttg

SEQ ID NO: 379 - DNA, 24 bp, Artificial sequence
  Synthetic oligonucleotide
ccgagtcgatttacacggacgaat

SEQ ID NO: 380 - DNA, 24 bp, Artificial sequence
  Synthetic oligonucleotide
cgggttgaactgccatacattcac

SEQ ID NO: 381 - DNA, 27 bp, Artificial sequence
  Synthetic oligonucleotide
cctggaacaataactccaataccacgg

SEQ ID NO: 382 - DNA, 22 bp, Artificial sequence
  Synthetic oligonucleotide
tgtacgttgtacccatgtcgca

SEQ ID NO: 383 - DNA, 24 bp, Artificial sequence
  Synthetic oligonucleotide
cgaacctggttgttctttcgttgc

SEQ ID NO: 384 - DNA, 22 bp, Artificial sequence
  Synthetic oligonucleotide
acgatgcttggtaggttacgca

SEQ ID NO: 385 - DNA, 22 bp, Artificial sequence
  Synthetic oligonucleotide
acacgttgtagattggagccgt

SEQ ID NO: 386 - DNA, 20 bp, Artificial sequence
  Synthetic oligonucleotide
cctgctcgagtatctggtga

SEQ ID NO: 387 - DNA, 24 bp, Artificial sequence
  Synthetic oligonucleotide
tgcatacctacatcaccatgaccg

SEQ ID NO: 388 - DNA, 22 bp, Artificial sequence
  Synthetic oligonucleotide
tccctttgtagccacaccactt

SEQ ID NO: 389 - DNA, 22 bp, Artificial sequence
  Synthetic oligonucleotide
tcctgctcgagtatctggtgat

SEQ ID NO: 390 - DNA, 27 bp, Artificial sequence
  Synthetic oligonucleotide
cctcaaagcttacttctaacccactca

SEQ ID NO: 391 - DNA, 22 bp, Artificial sequence
  Synthetic oligonucleotide
ttcccatgagcaacccaaagtc

SEQ ID NO: 392 - DNA, 24 bp, Artificial sequence
  Synthetic oligonucleotide
agttcttgctgactctctcccaag

SEQ ID NO: 393 - DNA, 24 bp, Artificial sequence
  Synthetic oligonucleotide
ttcttgtattgggagcaggacctg

SEQ ID NO: 394 - DNA, 24 bp, Artificial sequence
  Synthetic oligonucleotide
agcagcttgctcagtagtagctgt

SEQ ID NO: 395 - DNA, 22 bp, Artificial sequence
  Synthetic oligonucleotide
tacgcatttcaccgctacacca

SEQ ID NO: 396 - DNA, 22 bp, Artificial sequence
  Synthetic oligonucleotide
accttctggagcttgccattgt

SEQ ID NO: 397 - DNA, 22 bp, Artificial sequence
  Synthetic oligonucleotide
gggtgcgcatagaaattgcatc

SEQ ID NO: 398 - DNA, 22 bp, Artificial sequence
  Synthetic oligonucleotide
tacgcatttcaccgctacacca

SEQ ID NO: 399 - DNA, 24 bp, Artificial sequence
  Synthetic oligonucleotide
tgagtcaagagcaatgtagctccc

SEQ ID NO: 400 - DNA, 24 bp, Artificial sequence
  Synthetic oligonucleotide
gcaggagagaggcatggtcatatt

SEQ ID NO: 401 - DNA, 24 bp, Artificial sequence
  Synthetic oligonucleotide
ggcctgccctcaatatctatccat

SEQ ID NO: 402 - DNA, 24 bp, Artificial sequence
  Synthetic oligonucleotide
tctgacaatgtccaggtgaaggtc

SEQ ID NO: 403 - DNA, 23 bp, Artificial sequence
  Synthetic oligonucleotide
tggtgttggtcaaggtcagttct

SEQ ID NO: 404 - DNA, 24 bp, Artificial sequence
  Synthetic oligonucleotide
tcagaaccttgacagctcaagacc

SEQ ID NO: 405 - DNA, 24 bp, Artificial sequence
  Synthetic oligonucleotide
cagtgtgcatcttgcataaccagc

SEQ ID NO: 406 - DNA, 24 bp, Artificial sequence
  Synthetic oligonucleotide
gttctacactggctctgagcactt

SEQ ID NO: 407 - DNA, 24 bp, Artificial sequence
  Synthetic oligonucleotide
tagttgggatgagaaagcctcagc

SEQ ID NO: 408 - DNA, 24 bp, Artificial sequence
  Synthetic oligonucleotide
atgtgggagacctcaacactaagc

SEQ ID NO: 409 - DNA, 24 bp, Artificial sequence
  Synthetic oligonucleotide
ggttcctatcacactctttgggct

SEQ ID NO: 410 - DNA, 24 bp, Artificial sequence
  Synthetic oligonucleotide
cgatgacactcttgggactgacaa

SEQ ID NO: 411 - DNA, 24 bp, Artificial sequence
  Synthetic oligonucleotide
caaataaggccacagttggtgcag

SEQ ID NO: 412 - DNA, 22 bp, Artificial sequence
  Synthetic oligonucleotide
aatgccgtgtgagtgcctactt

SEQ ID NO: 413 - DNA, 22 bp, Artificial sequence
  Synthetic oligonucleotide
cgtggagttggcttccttgttt

SEQ ID NO: 414 - DNA, 24 bp, Artificial sequence
  Synthetic oligonucleotide
cacagcagattcttggatccctca

SEQ ID NO: 415 - DNA, 22 bp, Artificial sequence
  Synthetic oligonucleotide
tgaatgggaatggtgtcgggaa

SEQ ID NO: 416 - DNA, 22 bp, Artificial sequence
  Synthetic oligonucleotide
cttggcacacggattgttggtt

SEQ ID NO: 417 - DNA, 22 bp, Artificial sequence
  Synthetic oligonucleotide
tgcattgtcattccagcttggc

SEQ ID NO: 418 - DNA, 22 bp, Artificial sequence
  Synthetic oligonucleotide
acggctgtaacggactgtgatt

SEQ ID NO: 419 - DNA, 22 bp, Artificial sequence
  Synthetic oligonucleotide
aaccacccttccctgacacttt

SEQ ID NO: 420 - DNA, 24 bp, Artificial sequence
  Synthetic oligonucleotide
cccagatcagtatgcattgggaca

SEQ ID NO: 421 - DNA, 22 bp, Artificial sequence
  Synthetic oligonucleotide
tgcaaggctcaactctctggat

SEQ ID NO: 422 - DNA, 24 bp, Artificial sequence
  Synthetic oligonucleotide
agtctgaccagagtccattgttcc

SEQ ID NO: 423 - DNA, 24 bp, Artificial sequence
  Synthetic oligonucleotide
tcaggtctctcctgtggaagtaca

SEQ ID NO: 424 - DNA, 24 bp, Artificial sequence
  Synthetic oligonucleotide
tgcctggaatgatgctgtagagac

SEQ ID NO: 425 - DNA, 22 bp, Artificial sequence
  Synthetic oligonucleotide
tttgtccacatctccacgtcca

SEQ ID NO: 426 - DNA, 24 bp, Artificial sequence
  Synthetic oligonucleotide
aagagatcccaatagcaccggaag

SEQ ID NO: 427 - DNA, 22 bp, Artificial sequence
  Synthetic oligonucleotide
ttcgcgtcttgttcttccacca

SEQ ID NO: 428 - DNA, 22 bp, Artificial sequence
  Synthetic oligonucleotide
tcgatgccaccagaaaccagaa

SEQ ID NO: 429 - DNA, 24 bp, Artificial sequence
  Synthetic oligonucleotide
gcaatttacggcgacggtcaatac

SEQ ID NO: 430 - DNA, 22 bp, Artificial sequence
  Synthetic oligonucleotide
aaacacggttccagacctccaa

SEQ ID NO: 431 - DNA, 24 bp, Artificial sequence
  Synthetic oligonucleotide
ccaccacgatgtaagagtcaccaa

SEQ ID NO: 432 - DNA, 24 bp, Artificial sequence
  Synthetic oligonucleotide
cgtccttcatgatcagttccgtga

SEQ ID NO: 433 - DNA, 22 bp, Artificial sequence
  Synthetic oligonucleotide
tcatgacaaattgctggcgctg

SEQ ID NO: 434 - DNA, 24 bp, Artificial sequence
  Synthetic oligonucleotide
cacactctgcatcgtcctcttctt

SEQ ID NO: 435 - DNA, 22 bp, Artificial sequence
  Synthetic oligonucleotide
gcaaacagcctgaaggaagcaa

SEQ ID NO: 436 - DNA, 22 bp, Artificial sequence
  Synthetic oligonucleotide
gcacggtggcagcattgttatt

SEQ ID NO: 437 - DNA, 23 bp, Artificial sequence
  Synthetic oligonucleotide
attgcagcagtacgcacacaatc

SEQ ID NO: 438 - DNA, 25 bp, Artificial sequence
  Synthetic oligonucleotide
tagtagtcgtcgtcggctcatcata

SEQ ID NO: 439 - DNA, 24 bp, Artificial sequence
  Synthetic oligonucleotide
cgaatagcttcttcgcgggtgata

SEQ ID NO: 440 - DNA, 22 bp, Artificial sequence
  Synthetic oligonucleotide
aagcagttgtagcatcaccgga

SEQ ID NO: 441 - DNA, 57 bp, Artificial sequence
  Synthetic oligonucleotide
caaccgccaatgatgcgcacaatgataatgaaccatctactgtgtcgccaacaactg

SEQ ID NO: 442 - DNA, 57 bp, Artificial sequence
  Synthetic oligonucleotide
atctctcatctgtattcagagtcggatacgagagcatatgtgcgtccggaagttgtt

SEQ ID NO: 443 - DNA, 57 bp, Artificial sequence
  Synthetic oligonucleotide
aggtaaggactctcccgctatcactcgtgtagaagctctggctatgatcaaagactg

SEQ ID NO: 444 - DNA, 57 bp, Artificial sequence
  Synthetic oligonucleotide
caaatctcgcgttgaacgagtgcaagtagaacttactgacaaagttaaggtgcgagt

SEQ ID NO: 445 - DNA, 57 bp, Artificial sequence
  Synthetic oligonucleotide
ggcattagaacctaattacgacgtagaaagtcgcggcgaagagcttccgctatctac

SEQ ID NO: 446 - DNA, 59 bp, Artificial sequence
  Synthetic oligonucleotide
caaccgccaatgatgcgcacaatgataatgaaccatctactgtgtcgccaacaactgta

SEQ ID NO: 447 - DNA, 57 bp, Artificial sequence
  Synthetic oligonucleotide
acagtaagtacatcatctggagaatccacaacagacaagacgtcgggaccaattact

SEQ ID NO: 448 - DNA, 60 bp, Artificial sequence
  Synthetic oligonucleotide
tgcggatctttatgatacgcacaatgataatgaaccatctactgtgtcaccaacaactgt

SEQ ID NO: 449 - DNA, 55 bp, Artificial sequence
  Synthetic oligonucleotide
ggatttgtggtgtcccataccactagatttcctcgtcctatggaacgagaaggtg

SEQ ID NO: 450 - DNA, 54 bp, Artificial sequence
  Synthetic oligonucleotide
ctacattcccgggagacgcggctatagctactacgtttacggtatagcctctag

SEQ ID NO: 451 - DNA, 54 bp, Artificial sequence
  Synthetic oligonucleotide
acagtaagtgcatcatctggagaatccacaacagacgagactccggaaccaatt

SEQ ID NO: 452 - DNA, 59 bp, Artificial sequence
  Synthetic oligonucleotide
tgctcgtcggtattcgaaatcgcgactccggaaccaattactgataatgtagaagatca

SEQ ID NO: 453 - DNA, 61 bp, Artificial sequence
  Synthetic oligonucleotide
acactacagtaagtacatcatctggaattgtcactactaaatcaaccaccgatgatgcgga

SEQ ID NO: 454 - DNA, 57 bp, Artificial sequence
  Synthetic oligonucleotide
attctcctgtttgatttctctatcgatgcggcacctctcttaagaagtgtaaccgat

SEQ ID NO: 455 - DNA, 56 bp, Artificial sequence
  Synthetic oligonucleotide
tcgatgactacgattgcacgtctacaggttgcagcatagactttgtcacaacagaa

SEQ ID NO: 456 - DNA, 56 bp, Artificial sequence
  Synthetic oligonucleotide
ttgaacaaggcgattataaagtggaagagtattgtacgggaccaccgactgtaaca

SEQ ID NO: 457 - DNA, 63 bp, Artificial sequence
  Synthetic oligonucleotide
ggaccaccgactgtaacattaactgaatacgacgaccatatcaatttgtacatcgagcatccg

SEQ ID NO: 458 - DNA, 56 bp, Artificial sequence
  Synthetic oligonucleotide
ctggtggatactctagtaaagtcaggactgacagaggtgttcggttcaactggaga

SEQ ID NO: 459 - DNA, 56 bp, Artificial sequence
  Synthetic oligonucleotide
ctatcccgaaagaactgattgcagtgttcatctcccaactgcaagtgaaggattga

SEQ ID NO: 460 - DNA, 60 bp, Artificial sequence
  Synthetic oligonucleotide
tcattgactgctagagatgccggtacttatgtatgtgcattctttatgacatcgcctaca

SEQ ID NO: 461 - DNA, 55 bp, Artificial sequence
  Synthetic oligonucleotide
ggagtaaatgttggagaacagtatcaacaactcagagaggctgccactgaggctg

SEQ ID NO: 462 - DNA, 55 bp, Artificial sequence
  Synthetic oligonucleotide
ccgaacatggtcaaccaccacctggaccatcactttatgaagaaagtgcgattcg

SEQ ID NO: 463 - DNA, 55 bp, Artificial sequence
  Synthetic oligonucleotide
gacctacagctttgactcaactacggccgccatcatgcttgcttcatacactatc

SEQ ID NO: 464 - DNA, 55 bp, Artificial sequence
  Synthetic oligonucleotide
gaagagagtgccagcagcgggaagctaggcttaattaccaatactattgctggag

SEQ ID NO: 465 - DNA, 54 bp, Artificial sequence
  Synthetic oligonucleotide
gatcgacttgcttccacagttatctaccgaggaacgactttcgctgaaggtgtc

SEQ ID NO: 466 - DNA, 55 bp, Artificial sequence
  Synthetic oligonucleotide
ggcttgggcaagatcaggcagaacccgttctcgaagtatatcaacgattacacag

SEQ ID NO: 467 - DNA, 55 bp, Artificial sequence
  Synthetic oligonucleotide
ctgattgaccagtctttgattgaacccttagcaggagcccttggtctgatctctg

SEQ ID NO: 468 - DNA, 55 bp, Artificial sequence
  Synthetic oligonucleotide
gcgatccatctctgagacacgacatatctttccttgcaggataaccgcagctttc

SEQ ID NO: 469 - DNA, 59 bp, Artificial sequence
  Synthetic oligonucleotide
ccgtcaatcaaggagcgcctcacaagtgcgcgttcctactgtatttcataagaagagag

SEQ ID NO: 470 - DNA, 54 bp, Artificial sequence
  Synthetic oligonucleotide
atcgtgtcagaaatgtccaaacactcgcagaagccttgctagcagatggactag

SEQ ID NO: 471 - DNA, 54 bp, Artificial sequence
  Synthetic oligonucleotide
ccgactcgcaatgttcaaacactttgtgaagctctgttagctgatggtcttgct

SEQ ID NO: 472 - DNA, 58 bp, Artificial sequence
  Synthetic oligonucleotide
gcgacttggaagaagcaatgacacagagttaaactatgtcagttgtgctctcgaccgg

SEQ ID NO: 473 - DNA, 57 bp, Artificial sequence
  Synthetic oligonucleotide
ggtctgcaggttgaggcgtctagccaatcaaactgccaaatccttggaactcttatt

SEQ ID NO: 474 - DNA, 57 bp, Artificial sequence
  Synthetic oligonucleotide
atgtcaaaggcgacaagcactgatgatattgtttgggaccaactgatcgtgaagaaa

SEQ ID NO: 475 - DNA, 59 bp, Artificial sequence
  Synthetic oligonucleotide
cctttagctcatactgtggctgcgttgctcacaggcagctatacaatcacccaatttac

SEQ ID NO: 476 - DNA, 55 bp, Artificial sequence
  Synthetic oligonucleotide
cgtaaagaaggtcctaatatcggtgagcaacgcagggtagttaaagaggctgctg

SEQ ID NO: 477 - DNA, 58 bp, Artificial sequence
  Synthetic oligonucleotide
ccgtggtattggagttattgttccaggattaattgcaaatacaattcaaagacaaggg

SEQ ID NO: 478 - DNA, 57 bp, Artificial sequence
  Synthetic oligonucleotide
cgcagttatattaatagctgcgacatgggtacaacgtacagaagcagtagcaccagt

SEQ ID NO: 479 - DNA, 60 bp, Artificial sequence
  Synthetic oligonucleotide
ggtgttagggttgctactcttggatttacagatgcatttgtagcaggagctattgcaacg

SEQ ID NO: 480 - DNA, 54 bp, Artificial sequence
  Synthetic oligonucleotide
gtcgttcaatctgtaagccctgtcgtcgaacagtacggtcccattatgcgtaac

SEQ ID NO: 481 - DNA, 49 bp, Artificial sequence
  Synthetic oligonucleotide
actgggacggctccaatctacaacgtgttaccaacgacttcgttagtgt

SEQ ID NO: 482 - DNA, 61 bp, Artificial sequence
  Synthetic oligonucleotide
ggcctgcattagataatgagcgtttgaaatggagaatccaattatcaccagatactcgagc

SEQ ID NO: 483 - DNA, 37 bp, Artificial sequence
  Synthetic oligonucleotide
gggcgggcggtcatggtgatgtaggtatgcacgtaaa

SEQ ID NO: 484 - DNA, 53 bp, Artificial sequence
  Synthetic oligonucleotide
aagtggtgtggctacaaagggattgaatgaacatggaaagagttcggattggg

SEQ ID NO: 485 - DNA, 61 bp, Artificial sequence; identical to SEQ ID NO: 482
  Synthetic oligonucleotide

SEQ ID NO: 486 - DNA, 62 bp, Artificial sequence
  Synthetic oligonucleotide
ggtgcaggcaaatttgctacagatccagcagtaacattagcacatgaacttatacatgctgg

SEQ ID NO: 487 - DNA, 55 bp, Artificial sequence
  Synthetic oligonucleotide
tctagtaggactttgggttgctcatgggaatttattcctgtagatgatggatggg

SEQ ID NO: 488 - DNA, 57 bp, Artificial sequence
  Synthetic oligonucleotide
tggatcaatcttgggagagagtcagcaagaactaaattctatggtaactgataccct

SEQ ID NO: 489 - DNA, 54 bp, Artificial sequence
  Synthetic oligonucleotide
attgcagatcctgcaatttcactagctcatgaattgatacatgcactgcatgga

SEQ ID NO: 490 - DNA, 54 bp, Artificial sequence
  Synthetic oligonucleotide
cttcagctaaagatactgctgctgctcagacagctactactgagcaagctgctg

SEQ ID NO: 491 - DNA, 55 bp, Artificial sequence
  Synthetic oligonucleotide
cagggctcaaccttggaactgcatttgatactggcaaactagagtacggtagagg

SEQ ID NO: 492 - DNA, 53 bp, Artificial sequence
  Synthetic oligonucleotide
gctacgcaagctactgctaagcaaacaggtgtatctaagccaactgcaaaggt

SEQ ID NO: 493 - DNA, 54 bp, Artificial sequence
  Synthetic oligonucleotide
ccagtcactatggcgtgctgctagcgctatatgcgttgatgcaatttctatgcg

SEQ ID NO: 494 - DNA, 58 bp, Artificial sequence
  Synthetic oligonucleotide
gcaaattaggcgaacaagcagggtatgctaatatagttgattgcgtattgtatgtcga

SEQ ID NO: 495 - DNA, 54 bp, Artificial sequence
  Synthetic oligonucleotide
gagctacattgctcttgactcaggccgtggcaactgggactgtattatgactag

SEQ ID NO: 496 - DNA, 55 bp, Artificial sequence
  Synthetic oligonucleotide
cttgttggtttggtcactttcctcctgttgtgtggtaggtcttgcacaaccagtc

SEQ ID NO: 497 - DNA, 54 bp, Artificial sequence
  Synthetic oligonucleotide
gcttgcaggctgcaggtctaaatgctgggttgacctattctcaactgatgacac

SEQ ID NO: 498 - DNA, 55 bp, Artificial sequence
  Synthetic oligonucleotide
aaatacaacatgggaggaccactgccaattctcaagaccgtctcctatcgggtac

SEQ ID NO: 499 - DNA, 55 bp, Artificial sequence
  Synthetic oligonucleotide
tacataagggtgggcaatgagacaggactagaactgaccttgaccaacaccagta

SEQ ID NO: 500 - DNA, 55 bp, Artificial sequence
  Synthetic oligonucleotide
atggatcttgctgacctcttcaatgcacaggctgggctgacctcatcagttatag

SEQ ID NO: 501 - DNA, 55 bp, Artificial sequence
  Synthetic oligonucleotide
gatgttgagatgaccaaagaggcttcaagagagtatgaagacaaagtgtgggaca

SEQ ID NO: 502 - DNA, 55 bp, Artificial sequence
  Synthetic oligonucleotide
ggcagtatcctgcgacttcaacaatggcataaccatccaatacaacttgacattc

SEQ ID NO: 503 - DNA, 55 bp, Artificial sequence
  Synthetic oligonucleotide
cgtcattgagcggagtctgtgactgtttggccatacaagccatagttagacttgg

SEQ ID NO: 504 - DNA, 55 bp, Artificial sequence
  Synthetic oligonucleotide
ctcattgaactacccacagcttctgagaagtcttcaactaacctggtcatcagct

SEQ ID NO: 505 - DNA, 56 bp, Artificial sequence
  Synthetic oligonucleotide
agtgtcaagacagaacagaactgctggaaatggtgtgcttccatgaattcttatca

SEQ ID NO: 506 - DNA, 55 bp, Artificial sequence
  Synthetic oligonucleotide
cagatgctgcggagtggctcgagatgatctgcttccatgagtttctgtcatctaa

SEQ ID NO: 507 - DNA, 55 bp, Artificial sequence
  Synthetic oligonucleotide
gggccctagaagatgatgagagtgttgtttctatgctgcaccaactgtggcctta

SEQ ID NO: 508 - DNA, 55 bp, Artificial sequence
  Synthetic oligonucleotide
ccgtggagttggcagtgtttcagccttcttcaggaaactatgtacactgcttcag

SEQ ID NO: 509 - DNA, 53 bp, Artificial sequence
  Synthetic oligonucleotide
aaatttggccatctctgcagagcacacaatggtgtcattgttcccaagaagaa

SEQ ID NO: 510 - DNA, 55 bp, Artificial sequence
  Synthetic oligonucleotide
ggctctccagagtttgattggattcttgggtggacaattaagggattgggacatg

SEQ ID NO: 511 - DNA, 55 bp, Artificial sequence
  Synthetic oligonucleotide
ctaactccacgaaggagccacactgtgctcttctcgattgcatcatgtttcagtc

SEQ ID NO: 512 - DNA, 55 bp, Artificial sequence
  Synthetic oligonucleotide
gggatctggacacaccaagtccattcttaacctacggacaaacaccgaaaccaac

SEQ ID NO: 513 - DNA, 54 bp, Artificial sequence
  Synthetic oligonucleotide
gaatcaatcatgtgggcagctagtgcatcagaaactgtcttggagccaagctgg

SEQ ID NO: 514 - DNA, 55 bp, Artificial sequence
  Synthetic oligonucleotide
gtacgggctgtactgcatgcggactatacattgaccaacttaaacctgtaggcag

SEQ ID NO: 515 - DNA, 55 bp, Artificial sequence
  Synthetic oligonucleotide
caagaggccagaatacagtcaaagtgtcagggaagggtggttatagtggctcaac

SEQ ID NO: 516 - DNA, 55 bp, Artificial sequence
  Synthetic oligonucleotide
ccaagctggaatgacaacgcacatggtgttggtgttgtcccaatgcatactgatc

SEQ ID NO: 517 - DNA, 57 bp, Artificial sequence
  Synthetic oligonucleotide
gcctttatgtgtagggtatgagagagtggttgtgaagagagaactctctgccaagcc

SEQ ID NO: 518 - DNA, 55 bp, Artificial sequence
  Synthetic oligonucleotide
ctcagcactgcacatgaggttgtgccctttgcagtgtttaagaactcaaagaagg

SEQ ID NO: 519 - DNA, 55 bp, Artificial sequence
  Synthetic oligonucleotide
gcagctgatgtacttccacaggagagacctgagactagctgctaatgctatctgt

SEQ ID NO: 520 - DNA, 52 bp, Artificial sequence
  Synthetic oligonucleotide
ccggcataacaataaacagcatattgacgctgggagagaccagagatcctgc

SEQ ID NO: 521 - DNA, 54 bp, Artificial sequence
  Synthetic oligonucleotide
caaagttgcctcagaaggcttccagtactctgacagaagatggtgctttgacgg

SEQ ID NO: 522 - DNA, 55 bp, Artificial sequence
  Synthetic oligonucleotide
gactgactttcagtcacatcagctgtgggctaccttgctgtccttgacatttgtc

SEQ ID NO: 523 - DNA, 55 bp, Artificial sequence
  Synthetic oligonucleotide
cgattcaagatgtccaacacaaggagaagctacactggtggaagaacaagacgcg

SEQ ID NO: 524 - DNA, 55 bp, Artificial sequence
  Synthetic oligonucleotide
caactgaaacaggctaagaaagacctgactgaagagttgcagatcctggaagcgg

SEQ ID NO: 525 - DNA, 55 bp, Artificial sequence
  Synthetic oligonucleotide
caacgcacgtatcatcccttaccgcggttcatggttagatttcgagtttgatccg

SEQ ID NO: 526 - DNA, 50 bp, Artificial sequence
  Synthetic oligonucleotide
gaagaaccacgtggtccatccatgcaggaggagagtggatgacaacagag

SEQ ID NO: 527 - DNA, 53 bp, Artificial sequence
  Synthetic oligonucleotide
atgacctcacacctgttggaagactggtgaccgtgaatccatttgtgtctgtg

SEQ ID NO: 528 - DNA, 53 bp, Artificial sequence
  Synthetic oligonucleotide
aaatgctatgtcaaaggtccgcaaagacatccaggaatggaaaccctcgacgg

SEQ ID NO: 529 - DNA, 56 bp, Artificial sequence
  Synthetic oligonucleotide
catggtgttgtcttcctacatgtcacgtatgtgccatcccaggagaggaacttcac

SEQ ID NO: 530 - DNA, 58 bp, Artificial sequence
  Synthetic oligonucleotide
accagtttctgatctccttaccaacatgggtattgatcttgatgagtggagtgtagct

SEQ ID NO: 531 - DNA, 58 bp, Artificial sequence
  Synthetic oligonucleotide
gtattgtaggcttgatgtggcttagctacttcgttgcttccttcaggctgtttgctcg

SEQ ID NO: 532 - DNA, 54 bp, Artificial sequence
  Synthetic oligonucleotide
catcgtatgggttgcaactgagggagccttgaatacacccaaagaccacattgg

SEQ ID NO: 533 - DNA, 55 bp, Artificial sequence
  Synthetic oligonucleotide
tttcttgctttcgtggtattcttgctagtcacactagccatccttactgcgcttc

SEQ ID NO: 534 - DNA, 56 bp, Artificial sequence
  Synthetic oligonucleotide
aaatacacacaatcgacggctcttcaggagttgctaatccagcaatggatccaatt

SEQ ID NO: 535 - DNA, 58 bp, Artificial sequence
  Synthetic oligonucleotide
tgacataccaggcataccaaaggacatgacctaccgtagactcatctctatgatgggt

SEQ ID NO: 536 - DNA, 55 bp, Artificial sequence
  Synthetic oligonucleotide
aagtgagatggtcatgtgtggcggctcactatatgttaaaccaggtggaacatca

SEQ ID NO: 537 - DNA, 57 bp, Artificial sequence
  Synthetic oligonucleotide
cagttgttggcgacacagtagatggttcattatcattgtgcgcatcattggcggttg

SEQ ID NO: 538 - DNA, 57 bp, Artificial sequence
  Synthetic oligonucleotide
aacaacttccggacgcacatatgctctcgtatccgactctgaatacagatgagagat

SEQ ID NO: 539 - DNA, 57 bp, Artificial sequence
  Synthetic oligonucleotide
cagtctttgatcatagccagagcttctacacgagtgatagcgggagagtccttacct

SEQ ID NO: 540 - DNA, 57 bp, Artificial sequence
  Synthetic oligonucleotide
actcgcaccttaactttgtcagtaagttctacttgcactcgttcaacgcgagatttg

SEQ ID NO: 541 - DNA, 57 bp, Artificial sequence
  Synthetic oligonucleotide
gtagatagcggaagctcttcgccgcgactttctacgtcgtaattaggttctaatgcc

SEQ ID NO: 542 - DNA, 59 bp, Artificial sequence
  Synthetic oligonucleotide
tacagttgttggcgacacagtagatggttcattatcattgtgcgcatcattggcggttg

SEQ ID NO: 543 - DNA, 57 bp, Artificial sequence
  Synthetic oligonucleotide
agtaattggtcccgacgtcttgtctgttgtggattctccagatgatgtacttactgt

SEQ ID NO: 544 - DNA, 60 bp, Artificial sequence
  Synthetic oligonucleotide
acagttgttggtgacacagtagatggttcattatcattgtgcgtatcataaagatccgca

SEQ ID NO: 545 - DNA, 55 bp, Artificial sequence
  Synthetic oligonucleotide
caccttctcgttccataggacgaggaaatctagtggtatgggacaccacaaatcc

SEQ ID NO: 546 - DNA, 54 bp, Artificial sequence
  Synthetic oligonucleotide
ctagaggctataccgtaaacgtagtagctatagccgcgtctcccgggaatgtag

SEQ ID NO: 547 - DNA, 54 bp, Artificial sequence
  Synthetic oligonucleotide
aattggttccggagtctcgtctgttgtggattctccagatgatgcacttactgt

SEQ ID NO: 548 - DNA, 59 bp, Artificial sequence
  Synthetic oligonucleotide
tgatcttctacattatcagtaattggttccggagtcgcgatttcgaataccgacgagca

SEQ ID NO: 549 - DNA, 61 bp, Artificial sequence
  Synthetic oligonucleotide
tccgcatcatcggtggttgatttagtagtgacaattccagatgatgtacttactgtagtgt

SEQ ID NO: 550 - DNA, 57 bp, Artificial sequence
  Synthetic oligonucleotide
atcggttacacttcttaagagaggtgccgcatcgatagagaaatcaaacaggagaat

SEQ ID NO: 551 - DNA, 56 bp, Artificial sequence
  Synthetic oligonucleotide
ttctgttgtgacaaagtctatgctgcaacctgtagacgtgcaatcgtagtcatcga

SEQ ID NO: 552 - DNA, 56 bp, Artificial sequence
  Synthetic oligonucleotide
tgttacagtcggtggtcccgtacaatactcttccactttataatcgccttgttcaa

SEQ ID NO: 553 - DNA, 63 bp, Artificial sequence
  Synthetic oligonucleotide
cggatgctcgatgtacaaattgatatggtcgtcgtattcagttaatgttacagtcggtggtcc

SEQ ID NO: 554 - DNA, 56 bp, Artificial sequence
  Synthetic oligonucleotide
tctccagttgaaccgaacacctctgtcagtcctgactttactagagtatccaccag

SEQ ID NO: 555 - DNA, 56 bp, Artificial sequence
  Synthetic oligonucleotide
tcaatccttcacttgcagttgggagatgaacactgcaatcagttctttcgggatag

SEQ ID NO: 556 - DNA, 60 bp, Artificial sequence
  Synthetic oligonucleotide
tgtaggcgatgtcataaagaatgcacatacataagtaccggcatctctagcagtcaatga

SEQ ID NO: 557 - DNA, 55 bp, Artificial sequence
  Synthetic oligonucleotide
cagcctcagtggcagcctctctgagttgttgatactgttctccaacatttactcc

SEQ ID NO: 558 - DNA, 55 bp, Artificial sequence
  Synthetic oligonucleotide
cgaatcgcactttcttcataaagtgatggtccaggtggtggttgaccatgttcgg

SEQ ID NO: 559 - DNA, 55 bp, Artificial sequence
  Synthetic oligonucleotide
gatagtgtatgaagcaagcatgatggcggccgtagttgagtcaaagctgtaggtc

SEQ ID NO: 560 - DNA, 55 bp, Artificial sequence
  Synthetic oligonucleotide
ctccagcaatagtattggtaattaagcctagcttcccgctgctggcactctcttc

SEQ ID NO: 561 - DNA, 54 bp, Artificial sequence
  Synthetic oligonucleotide
gacaccttcagcgaaagtcgttcctcggtagataactgtggaagcaagtcgatc

SEQ ID NO: 562 - DNA, 55 bp, Artificial sequence
  Synthetic oligonucleotide
ctgtgtaatcgttgatatacttcgagaacgggttctgcctgatcttgcccaagcc

SEQ ID NO: 563 - DNA, 55 bp, Artificial sequence
  Synthetic oligonucleotide
cagagatcagaccaagggctcctgctaagggttcaatcaaagactggtcaatcag

SEQ ID NO: 564 - DNA, 55 bp, Artificial sequence
  Synthetic oligonucleotide
gaaagctgcggttatcctgcaaggaaagatatgtcgtgtctcagagatggatcgc

SEQ ID NO: 565 - DNA, 59 bp, Artificial sequence
  Synthetic oligonucleotide
ctctcttcttatgaaatacagtaggaacgcgcacttgtgaggcgctccttgattgacgg

SEQ ID NO: 566 - DNA, 54 bp, Artificial sequence
  Synthetic oligonucleotide
ctagtccatctgctagcaaggcttctgcgagtgtttggacatttctgacacgat

SEQ ID NO: 567 - DNA, 54 bp, Artificial sequence
  Synthetic oligonucleotide
agcaagaccatcagctaacagagcttcacaaagtgtttgaacattgcgagtcgg

SEQ ID NO: 568 - DNA, 58 bp, Artificial sequence
  Synthetic oligonucleotide
ccggtcgagagcacaactgacatagtttaactctgtgtcattgcttcttccaagtcgc

SEQ ID NO: 569 - DNA, 57 bp, Artificial sequence
  Synthetic oligonucleotide
aataagagttccaaggatttggcagtttgattggctagacgcctcaacctgcagacc

SEQ ID NO: 570 - DNA, 57 bp, Artificial sequence
  Synthetic oligonucleotide
tttcttcacgatcagttggtcccaaacaatatcatcagtgcttgtcgcctttgacat

SEQ ID NO: 571 - DNA, 59 bp, Artificial sequence
  Synthetic oligonucleotide
gtaaattgggtgattgtatagctgcctgtgagcaacgcagccacagtatgagctaaagg

SEQ ID NO: 572 - DNA, 55 bp, Artificial sequence
  Synthetic oligonucleotide
cagcagcctctttaactaccctgcgttgctcaccgatattaggaccttctttacg

SEQ ID NO: 573 - DNA, 58 bp, Artificial sequence
  Synthetic oligonucleotide
cccttgtctttgaattgtatttgcaattaatcctggaacaataactccaataccacgg

SEQ ID NO: 574 - DNA, 57 bp, Artificial sequence
  Synthetic oligonucleotide
actggtgctactgcttctgtacgttgtacccatgtcgcagctattaatataactgcg

SEQ ID NO: 575 - DNA, 60 bp, Artificial sequence
  Synthetic oligonucleotide
cgttgcaatagctcctgctacaaatgcatctgtaaatccaagagtagcaaccctaacacc

SEQ ID NO: 576 - DNA, 54 bp, Artificial sequence
  Synthetic oligonucleotide
gttacgcataatgggaccgtactgttcgacgacagggcttacagattgaacgac

SEQ ID NO: 577 - DNA, 49 bp, Artificial sequence
  Synthetic oligonucleotide
acactaacgaagtcgttggtaacacgttgtagattggagccgtcccagt

SEQ ID NO: 578 - DNA, 61 bp, Artificial sequence
  Synthetic oligonucleotide
gctcgagtatctggtgataattggattctccatttcaaacgctcattatctaatgcaggcc

SEQ ID NO: 579 - DNA, 37 bp, Artificial sequence
  Synthetic oligonucleotide
tttacgtgcatacctacatcaccatgaccgcccgccc

SEQ ID NO: 580 - DNA, 53 bp, Artificial sequence
  Synthetic oligonucleotide
cccaatccgaactctttccatgttcattcaatccctttgtagccacaccactt

SEQ ID NO: 581 - DNA, 61 bp, Artificial sequence; identical to SEQ ID NO: 578
  Synthetic oligonucleotide

SEQ ID NO: 582 - DNA, 62 bp, Artificial sequence; identical to SEQ ID NO: 486
  Synthetic oligonucleotide

SEQ ID NO: 583 - DNA, 55 bp, Artificial sequence
  Synthetic oligonucleotide
cccatccatcatctacaggaataaattcccatgagcaacccaaagtcctactaga

SEQ ID NO: 584 - DNA, 57 bp, Artificial sequence
  Synthetic oligonucleotide
agggtatcagttaccatagaatttagttcttgctgactctctcccaagattgatcca

SEQ ID NO: 585 - DNA, 54 bp, Artificial sequence
  Synthetic oligonucleotide
tccatgcagtgcatgtatcaattcatgagctagtgaaattgcaggatctgcaat

SEQ ID NO: 586 - DNA, 54 bp, Artificial sequence
  Synthetic oligonucleotide
cagcagcttgctcagtagtagctgtctgagcagcagcagtatctttagctgaag

SEQ ID NO: 587 - DNA, 55 bp, Artificial sequence
  Synthetic oligonucleotide
cctctaccgtactctagtttgccagtatcaaatgcagttccaaggttgagccctg

SEQ ID NO: 588 - DNA, 53 bp, Artificial sequence
  Synthetic oligonucleotide
acctttgcagttggcttagatacacctgtttgcttagcagtagcttgcgtagc

SEQ ID NO: 589 - DNA, 54 bp, Artificial sequence
  Synthetic oligonucleotide
cgcatagaaattgcatcaacgcatatagcgctagcagcacgccatagtgactgg

SEQ ID NO: 590 - DNA, 58 bp, Artificial sequence
  Synthetic oligonucleotide
tcgacatacaatacgcaatcaactatattagcataccctgcttgttcgcctaatttgc

SEQ ID NO: 591 - DNA, 54 bp, Artificial sequence
  Synthetic oligonucleotide
ctagtcataatacagtcccagttgccacggcctgagtcaagagcaatgtagctc

SEQ ID NO: 592 - DNA, 55 bp, Artificial sequence
  Synthetic oligonucleotide
gactggttgtgcaagacctaccacacaacaggaggaaagtgaccaaaccaacaag

SEQ ID NO: 593 - DNA, 54 bp, Artificial sequence
  Synthetic oligonucleotide
gtgtcatcagttgagaataggtcaacccagcatttagacctgcagcctgcaagc

SEQ ID NO: 594 - DNA, 55 bp, Artificial sequence
  Synthetic oligonucleotide
gtacccgataggagacggtcttgagaattggcagtggtcctcccatgttgtattt

SEQ ID NO: 595 - DNA, 55 bp, Artificial sequence
  Synthetic oligonucleotide
tactggtgttggtcaaggtcagttctagtcctgtctcattgcccacccttatgta

SEQ ID NO: 596 - DNA, 55 bp, Artificial sequence
  Synthetic oligonucleotide
ctataactgatgaggtcagcccagcctgtgcattgaagaggtcagcaagatccat

SEQ ID NO: 597 - DNA, 55 bp, Artificial sequence
  Synthetic oligonucleotide
tgtcccacactttgtcttcatactctcttgaagcctctttggtcatctcaacatc

SEQ ID NO: 598 - DNA, 55 bp, Artificial sequence
  Synthetic oligonucleotide
gaatgtcaagttgtattggatggttatgccattgttgaagtcgcaggatactgcc

SEQ ID NO: 599 - DNA, 55 bp, Artificial sequence
  Synthetic oligonucleotide
ccaagtctaactatggcttgtatggccaaacagtcacagactccgctcaatgacg

SEQ ID NO: 600 - DNA, 55 bp, Artificial sequence
  Synthetic oligonucleotide
agctgatgaccaggttagttgaagacttctcagaagctgtgggtagttcaatgag

SEQ ID NO: 601 - DNA, 56 bp, Artificial sequence
  Synthetic oligonucleotide
tgataagaattcatggaagcacaccatttccagcagttctgttctgtcttgacact

SEQ ID NO: 602 - DNA, 55 bp, Artificial sequence
  Synthetic oligonucleotide
ttagatgacagaaactcatggaagcagatcatctcgagccactccgcagcatctg

SEQ ID NO: 603 - DNA, 55 bp, Artificial sequence
  Synthetic oligonucleotide
taaggccacagttggtgcagcatagaaacaacactctcatcatcttctagggccc

SEQ ID NO: 604 - DNA, 55 bp, Artificial sequence
  Synthetic oligonucleotide
ctgaagcagtgtacatagtttcctgaagaaggctgaaacactgccaactccacgg

SEQ ID NO: 605 - DNA, 53 bp, Artificial sequence
  Synthetic oligonucleotide
ttcttcttgggaacaatgacaccattgtgtgctctgcagagatggccaaattt

SEQ ID NO: 606 - DNA, 55 bp, Artificial sequence
  Synthetic oligonucleotide
catgtcccaatcccttaattgtccacccaagaatccaatcaaactctggagagcc

SEQ ID NO: 607 - DNA, 55 bp, Artificial sequence
  Synthetic oligonucleotide
gactgaaacatgatgcaatcgagaagagcacagtgtggctccttcgtggagttag

SEQ ID NO: 608 - DNA, 55 bp, Artificial sequence
  Synthetic oligonucleotide
gttggtttcggtgtttgtccgtaggttaagaatggacttggtgtgtccagatccc

SEQ ID NO: 609 - DNA, 54 bp, Artificial sequence
  Synthetic oligonucleotide
ccagcttggctccaagacagtttctgatgcactagctgcccacatgattgattc

SEQ ID NO: 610 - DNA, 55 bp, Artificial sequence
  Synthetic oligonucleotide
ctgcctacaggtttaagttggtcaatgtatagtccgcatgcagtacagcccgtac

SEQ ID NO: 611 - DNA, 55 bp, Artificial sequence
  Synthetic oligonucleotide
gttgagccactataaccacccttccctgacactttgactgtattctggcctcttg

SEQ ID NO: 612 - DNA, 55 bp, Artificial sequence
  Synthetic oligonucleotide
gatcagtatgcattgggacaacaccaacaccatgtgcgttgtcattccagcttgg

SEQ ID NO: 613 - DNA, 57 bp, Artificial sequence
  Synthetic oligonucleotide
ggcttggcagagagttctctcttcacaaccactctctcataccctacacataaaggc

SEQ ID NO: 614 - DNA, 55 bp, Artificial sequence
  Synthetic oligonucleotide
ccttctttgagttcttaaacactgcaaagggcacaacctcatgtgcagtgctgag

SEQ ID NO: 615 - DNA, 55 bp, Artificial sequence
  Synthetic oligonucleotide
acagatagcattagcagctagtctcaggtctctcctgtggaagtacatcagctgc

SEQ ID NO: 616 - DNA, 52 bp, Artificial sequence
  Synthetic oligonucleotide
gcaggatctctggtctctcccagcgtcaatatgctgtttattgttatgccgg

SEQ ID NO: 617 - DNA, 54 bp, Artificial sequence
  Synthetic oligonucleotide
ccgtcaaagcaccatcttctgtcagagtactggaagccttctgaggcaactttg

SEQ ID NO: 618 - DNA, 55 bp, Artificial sequence
  Synthetic oligonucleotide
gacaaatgtcaaggacagcaaggtagcccacagctgatgtgactgaaagtcagtc

SEQ ID NO: 619 - DNA, 55 bp, Artificial sequence
  Synthetic oligonucleotide
cgcgtcttgttcttccaccagtgtagcttctccttgtgttggacatcttgaatcg

SEQ ID NO: 620 - DNA, 55 bp, Artificial sequence
  Synthetic oligonucleotide
ccgcttccaggatctgcaactcttcagtcaggtctttcttagcctgtttcagttg

SEQ ID NO: 621 - DNA, 55 bp, Artificial sequence
  Synthetic oligonucleotide
cggatcaaactcgaaatctaaccatgaaccgcggtaagggatgatacgtgcgttg

SEQ ID NO: 622 - DNA, 50 bp, Artificial sequence
  Synthetic oligonucleotide
ctctgttgtcatccactctcctcctgcatggatggaccacgtggttcttc

SEQ ID NO: 623 - DNA, 53 bp, Artificial sequence
  Synthetic oligonucleotide
cacagacacaaatggattcacggtcaccagtcttccaacaggtgtgaggtcat

SEQ ID NO: 624 - DNA, 53 bp, Artificial sequence
  Synthetic oligonucleotide
ccgtcgagggtttccattcctggatgtctttgcggacctttgacatagcattt

SEQ ID NO: 625 - DNA, 56 bp, Artificial sequence
  Synthetic oligonucleotide
gtgaagttcctctcctgggatggcacatacgtgacatgtaggaagacaacaccatg

SEQ ID NO: 626 - DNA, 58 bp, Artificial sequence
  Synthetic oligonucleotide
agctacactccactcatcaagatcaatacccatgttggtaaggagatcagaaactggt

SEQ ID NO: 627 - DNA, 58 bp, Artificial sequence
  Synthetic oligonucleotide
cgagcaaacagcctgaaggaagcaacgaagtagctaagccacatcaagcctacaatac

SEQ ID NO: 628 - DNA, 54 bp, Artificial sequence
  Synthetic oligonucleotide
ccaatgtggtctttgggtgtattcaaggctccctcagttgcaacccatacgatg

SEQ ID NO: 629 - DNA, 55 bp, Artificial sequence
  Synthetic oligonucleotide
gaagcgcagtaaggatggctagtgtgactagcaagaataccacgaaagcaagaaa

SEQ ID NO: 630 - DNA, 56 bp, Artificial sequence
  Synthetic oligonucleotide
aattggatccattgctggattagcaactcctgaagagccgtcgattgtgtgtattt

SEQ ID NO: 631 - DNA, 58 bp, Artificial sequence
  Synthetic oligonucleotide
acccatcatagagatgagtctacggtaggtcatgtcctttggtatgcctggtatgtca

SEQ ID NO: 632 - DNA, 55 bp, Artificial sequence
  Synthetic oligonucleotide
tgatgttccacctggtttaacatatagtgagccgccacacatgaccatctcactt

SEQ ID NO: 633 - DNA, 22 bp, Artificial sequence
  Synthetic oligonucleotide
atcgacaacgccctcagcatca

SEQ ID NO: 634 - DNA, 24 bp, Artificial sequence
  Synthetic oligonucleotide
ccaatcgctgggcttcgatttcaa

SEQ ID NO: 635 - DNA, 24 bp, Artificial sequence
  Synthetic oligonucleotide
ttgcagcagccactccaaagaaac

SEQ ID NO: 636 - DNA, 24 bp, Artificial sequence
  Synthetic oligonucleotide
agcacctacacccagaccaaatac

SEQ ID NO: 637 - DNA, 24 bp, Artificial sequence
  Synthetic oligonucleotide
tatacaaatgcctggagcggagca

SEQ ID NO: 638 - DNA, 26 bp, Artificial sequence
  Synthetic oligonucleotide
acgatacctgggaaggcaagatctac

SEQ ID NO: 639 - DNA, 24 bp, Artificial sequence
  Synthetic oligonucleotide
atcagcgcactagatggctcagaa

SEQ ID NO: 640 - DNA, 24 bp, Artificial sequence
  Synthetic oligonucleotide
cgttcagttcgtggatgaacacct

SEQ ID NO: 641 - DNA, 24 bp, Artificial sequence
  Synthetic oligonucleotide
tctggtaggccaaggtgaaagtgt

SEQ ID NO: 642 - DNA, 24 bp, Artificial sequence
  Synthetic oligonucleotide
tcgtgagacctattacttgcggct

SEQ ID NO: 643 - DNA, 23 bp, Artificial sequence
  Synthetic oligonucleotide
cgacgatttcctccacctgttgc

SEQ ID NO: 644 - DNA, 24 bp, Artificial sequence
  Synthetic oligonucleotide
tcaatgtttctcgatgcaagcgcc

SEQ ID NO: 645 - DNA, 22 bp, Artificial sequence
  Synthetic oligonucleotide
tggcgatgacgggtgaaagtct

SEQ ID NO: 646 - DNA, 24 bp, Artificial sequence
  Synthetic oligonucleotide
aatcgtcaggcgagatcgaatgga

SEQ ID NO: 647 - DNA, 54 bp, Artificial sequence
  Synthetic oligonucleotide
cagttggtcgctgaactggctggtaccgatcggccacgagaagccctcgaacat

SEQ ID NO: 648 - DNA, 60 bp, Artificial sequence
  Synthetic oligonucleotide
catctaccgcacaaacgatgaaggcaaggccaaggccggcgacatcagcaacaccacttg

SEQ ID NO: 649 - DNA, 58 bp, Artificial sequence
  Synthetic oligonucleotide
gaagcctatcgcaaggctgacgaagctctgggcgctgctcagaaagctcagcagactg

SEQ ID NO: 650 - DNA, 55 bp, Artificial sequence
  Synthetic oligonucleotide
tgacggtgcccaggtctacgtcaccgaagtcagccagttggacacctcggaagtc

SEQ ID NO: 651 - DNA, 57 bp, Artificial sequence
  Synthetic oligonucleotide
acatggcgacgcctgtcccgagaacttcatcttccaaggtaatgccttcgtcggctt

SEQ ID NO: 652 - DNA, 56 bp, Artificial sequence
  Synthetic oligonucleotide
aagcatgacctggacatcaaacccacggtcatcagtcatcgcctgcactttcccga

SEQ ID NO: 653 - DNA, 57 bp, Artificial sequence
  Synthetic oligonucleotide
ttgattggccacaatgccgctgacgccgcagatgcagatccagttattgttgttgca

SEQ ID NO: 654 - DNA, 54 bp, Artificial sequence
  Synthetic oligonucleotide
atgttcgagggcttctcgtggccgatcggtaccagccagttcagcgaccaactg

SEQ ID NO: 655 - DNA, 60 bp, Artificial sequence
  Synthetic oligonucleotide
caagtggtgttgctgatgtcgccggccttggccttgccttcatcgtttgtgcggtagatg

SEQ ID NO: 656 - DNA, 58 bp, Artificial sequence
  Synthetic oligonucleotide
cagtctgctgagctttctgagcagcgcccagagcttcgtcagccttgcgataggcttc

SEQ ID NO: 657 - DNA, 55 bp, Artificial sequence
  Synthetic oligonucleotide
gacttccgaggtgtccaactggctgacttcggtgacgtagacctgggcaccgtca

SEQ ID NO: 658 - DNA, 57 bp, Artificial sequence
  Synthetic oligonucleotide
aagccgacgaaggcattaccttggaagatgaagttctcgggacaggcgtcgccatgt

SEQ ID NO: 659 - DNA, 56 bp, Artificial sequence
  Synthetic oligonucleotide
tcgggaaagtgcaggcgatgactgatgaccgtgggtttgatgtccaggtcatgctt

SEQ ID NO: 660 - DNA, 57 bp, Artificial sequence
  Synthetic oligonucleotide
tgcaacaacaataactggatctgcatctgcggcgtcagcggcattgtggccaatcaa

SEQ ID NO: 661 - DNA, 24 bp, Artificial sequence
  Synthetic oligonucleotide
agcgacctccaccagacattgaaa

SEQ ID NO: 662 - DNA, 24 bp, Artificial sequence
  Synthetic oligonucleotide
aggcaaagagaagagtggtgcaga

SEQ ID NO: 663 - DNA, 24 bp, Artificial sequence
  Synthetic oligonucleotide
tgaccctgaagttcatctgcacca

SEQ ID NO: 664 - DNA, 24 bp, Artificial sequence
  Synthetic oligonucleotide
attggacgaaccactgaattgccg

SEQ ID NO: 665 - DNA, 24 bp, Artificial sequence
  Synthetic oligonucleotide
tgtaactcgccttgatcgttggga

SEQ ID NO: 666 - DNA, 24 bp, Artificial sequence
  Synthetic oligonucleotide
aggccaccacttcaagaactctgt

SEQ ID NO: 667 - DNA, 24 bp, Artificial sequence
  Synthetic oligonucleotide
gctgcttgctttgttcaaactgcc

SEQ ID NO: 668 - DNA, 24 bp, Artificial sequence
  Synthetic oligonucleotide
ccctcagcaaattgttctgctgct

SEQ ID NO: 669 - DNA, 24 bp, Artificial sequence
  Synthetic oligonucleotide
tcttgtagttgccgtcgtccttga

SEQ ID NO: 670 - DNA, 24 bp, Artificial sequence
  Synthetic oligonucleotide
aacagacgggcacacactacttga

SEQ ID NO: 671 - DNA, 24 bp, Artificial sequence
  Synthetic oligonucleotide
tcctgcaactttatccgcctccat

SEQ ID NO: 672 - DNA, 24 bp, Artificial sequence
  Synthetic oligonucleotide
atcgtcttgagtccaacccggtaa

SEQ ID NO: 673 - DNA, 55 bp, Artificial sequence
  Synthetic oligonucleotide
aacctggacgctttatgggattgtctgaccggatgggtggagtacccgctcgttt

SEQ ID NO: 674 - DNA, 56 bp, Artificial sequence
  Synthetic oligonucleotide
tttgttccttgggttcttgggagcagcaggaagcactatgggcgcagcctcaatga

SEQ ID NO: 675 - DNA, 56 bp, Artificial sequence
  Synthetic oligonucleotide
acatgaagcagcacgacttcttcaagtccgccatgcccgaaggctacgtccaggag

SEQ ID NO: 676 - DNA, 56 bp, Artificial sequence
  Synthetic oligonucleotide
accagatctgagcctgggagctctctggctaactagggaacccactgcttaagcct

SEQ ID NO: 677 - DNA, 57 bp, Artificial sequence
  Synthetic oligonucleotide
aaacgacgagcgtgacaccacgatgcctgtagcaatggcaacaacgttgcgcaaact

SEQ ID NO: 678 - DNA, 56 bp, Artificial sequence
  Synthetic oligonucleotide
acctcgctctgctaatcctgttaccagtggctgctgccagtggcgataagtcgtgt

SEQ ID NO: 679 - DNA, 55 bp, Artificial sequence
  Synthetic oligonucleotide
aaacgagcgggtactccacccatccggtcagacaatcccataaagcgtccaggtt

SEQ ID NO: 680 - DNA, 56 bp, Artificial sequence
  Synthetic oligonucleotide
tcattgaggctgcgcccatagtgcttcctgctgctcccaagaacccaaggaacaaa

SEQ ID NO: 681 - DNA, 56 bp, Artificial sequence
  Synthetic oligonucleotide
ctcctggacgtagccttcgggcatggcggacttgaagaagtcgtgctgcttcatgt

SEQ ID NO: 682 - DNA, 56 bp, Artificial sequence
  Synthetic oligonucleotide
aggcttaagcagtgggttccctagttagccagagagctcccaggctcagatctggt

SEQ ID NO: 683 - DNA, 57 bp, Artificial sequence
  Synthetic oligonucleotide
agtttgcgcaacgttgttgccattgctacaggcatcgtggtgtcacgctcgtcgttt

SEQ ID NO: 684 - DNA, 56 bp, Artificial sequence
  Synthetic oligonucleotide
acacgacttatcgccactggcagcagccactggtaacaggattagcagagcgaggt

(19) United States
(12) Patent Application Publication
(10) Pub. No.: US 2006/0172325 A1
(43) Pub. Date: Aug. 3, 2006

(54) Detection of nucleic acids

(75) Inventors: Michael J. Brownstein, Rockville, MD (US); Charlie Xiang, Germantown, MD (US); Zhi-Qing Qi, Rockville, MD (US)

Correspondence Address: KLARQUIST SPARKMAN, LLP, 121 S.W. SALMON STREET, SUITE #1600, PORTLAND, OR, 97204-2988, US

(73) Assignee: The Government of the U.S.A. as represented by the Secretary of the Dept. of Health & Human Services

(21) Appl. No.: 11/299,025

(22) Filed: Dec. 9, 2005

Related U.S. Application Data
(60) Provisional application No. 60/635,239, filed on Dec. 9, 2004.

(51) Int. Cl. C12Q 1/68; C12M 1/34

(52) U.S. Cl. 435/6; 435/287.2

(57) ABSTRACT

Disclosed herein are oligonucleotide microarrays, wherein the microarrays comprise a plurality of target-specific oligonucleotide probes, including both sense and antisense probe pairs for each target nucleic acid. Such arrays are useful for high throughput detection of target nucleic acids in a sample, particularly when coupled to multiplex PCR. Also disclosed herein are methods of using the disclosed microarrays.

### CROSS REFERENCE TO RELATED APPLICATION

This application claims the benefit of U.S. Provisional Application No. 60/635,239, filed Dec. 9, 2004, which is incorporated herein by reference in its entirety.

### FIELD OF THE DISCLOSURE

This disclosure relates to high throughput, microarray-based methods of detecting target nucleic acids in a sample, and in particular to multiplex PCR coupled with microarrays for the qualitative identification of multiple target nucleic acids. It further relates to oligonucleotide microarrays for use in such methods.

### BACKGROUND

Molecular methods are commonly used to detect specific nucleic acids in a sample. For example, a PCR-based assay, with primers specific for a target sequence, can be used to detect genomic nucleic acids (or transcripts derived therefrom) of pathogens of interest.

Although PCR amplification followed by separation and characterization of DNA products by gel electrophoresis is a simple and sensitive method, this approach has a number of inherent shortcomings. Highly sensitive PCR amplification tends to generate nonspecific DNA products, which complicate interpretation of the results. Additionally, in a typical method for detecting pathogens in a sample, PCR reactions for each pathogen must be run separately from one another due to differences in amplification conditions. Furthermore, in cases where multiplex PCR coupled with a microarray is used for the qualitative detection of several pathogens, the generation of nonspecific DNA products can be a significant problem (see, e.g., Elnifro et al., Clin. Microbiol. Rev. 13:559-70, 2000).

Thus, there remains a need for the development of a rapid, high-throughput method for qualitative identification of multiple target nucleic acids that is sensitive, highly discriminating and robust.

### SUMMARY OF THE DISCLOSURE

A novel method for high throughput qualitative detection of multiple target nucleic acids (including rare targets) in a sample, based on multiplex PCR followed by microarray analysis, has been developed. In the microarrays described herein, both sense and antisense oligonucleotide probe pairs corresponding to the target nucleic acid are printed on the microarray. This has the advantage of enabling the detection of balanced versus unbalanced multiplex PCR reactions. In an unbalanced reaction, certain primers participate in side reactions that result in the depletion of dNTPs. This limits the number of primers that can be multiplexed. Prediction and control of side reactions is not generally possible, and they render multiplex results less reliable. In a balanced multiplex PCR reaction, signals from both the sense and antisense probes are the same; in an unbalanced reaction the signals can be different, but at least one target-specific probe still gives a relatively strong signal. By printing sense and antisense probes for each target nucleic acid and examining the microarray for concordance, reliability is improved.

This disclosure provides methods and arrays useful for the rapid, qualitative detection of one or more targets, such as pathogens, in a sample (e.g., an environmental or a biological sample). For instance, the array-based methods provided herein can be used to rapidly identify the presence of a pathogen in an environmental sample, such as suspect powder. The array-based methods provided herein can also be used to rapidly identify the presence of a pathogen in a biological sample, such as blood. The disclosed methods and arrays can also be used for the rapid, qualitative detection of rare mRNAs expressed in cells, including both pathogenic and non-pathogenic cells.

The foregoing and other features and advantages will become more apparent from the following detailed description of several embodiments, which proceeds with reference to the accompanying figures.

### BRIEF DESCRIPTION OF THE FIGURES

The patent or application file contains at least one figure executed in color.

FIG. 1 is a series of digital images captured from scans of an oligonucleotide microarray, showing detection of target KSHV and EBV nucleic acids in samples from BC-1 cells infected by both KSHV and EBV. Serial dilutions were used to prepare the indicated quantity of input sample DNA. Red spots are oligonucleotide probes representing different ORFs of the KSHV and EBV genomes, as listed in Tables 3 and 4. Sense and antisense probe pairs were spotted in duplicate, in rows, following the order listed in the respective Tables, with a blank row between the KSHV and EBV probes. Green and yellow spots are oligonucleotide probes representing human housekeeping genes and other control elements. After serial dilution of input DNA samples, some viral genes (both KSHV and EBV) are detectable using as little as 1 pg of input DNA.

FIG. 2 a digital image captured from a scan of an oligonucleotide microarray, showing detection of target KSHV and EBV nucleic acids in samples from BC-1 cells infected by both KSHV and EBV. Also illustrated are the effects of unbalanced multiplex PCR reactions. Red spots are oligonucleotide probes representing different ORFs of the KSHV and EBV genomes, as listed in Tables 3 and 4. Sense and antisense probe pairs were spotted in duplicate, in rows, following the order listed in the respective Tables, with a blank row between the KSHV and EBV probes. The ORF 7 sense probe detected target KSHV nucleic acid, while the antisense probe did not. The opposite was seen with the ORF 8 probe pair, the sense probe did not detect KSHV nucleic acid, while the antisense probe did.

FIG. 3 is a photograph of a stained agarose gel, showing detection of target KSHV and EBV nucleic acids in samples from BC-1 cells infected by both KSHV and EBV using a standard gel-based assay system. Serial dilutions were used to prepare the indicated quantity of input sample DNA. This method can only detect signals in samples containing 100 pg or greater of input sample DNA.

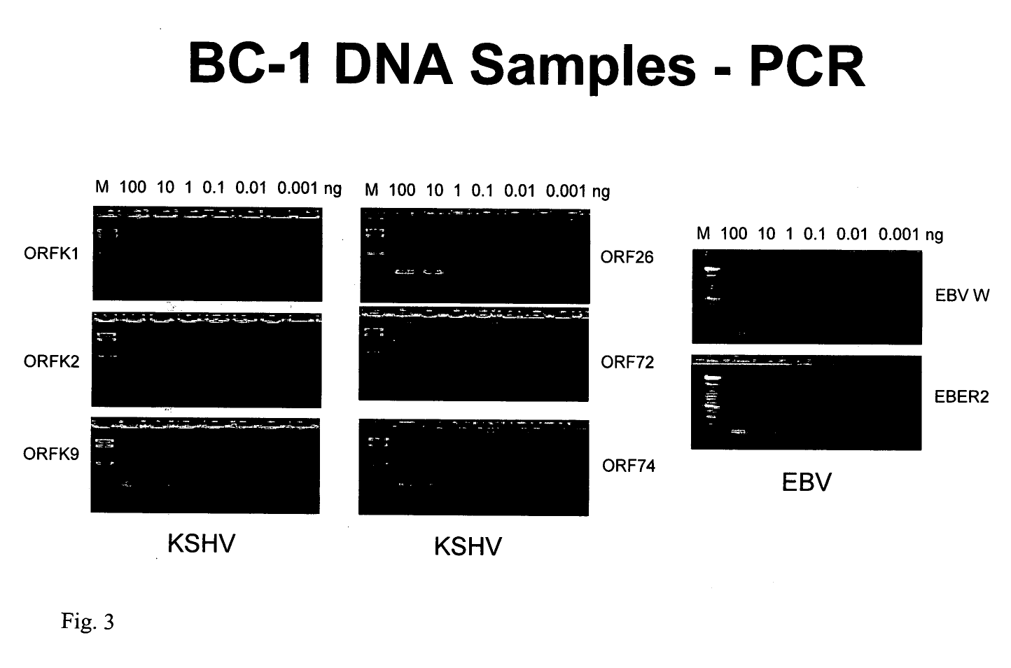

FIG. 4 is a series of digital images captured from scans of an oligonucleotide microarray, showing detection of target KSHV nucleic acids in samples from BCBL-1 cells infected by KSHV. Serial dilutions were used to prepare the indicated quantity of input sample DNA. Red spots are oligonucleotide probes representing different ORFs of the KSHV genome, as listed in Table 3. Sense and antisense probe pairs were spotted in duplicate, in rows, following the order listed in the Table. Green and yellow spots are oligonucleotide probes representing human house-keeping genes and other control elements. After serial dilution of input DNA samples, some KSHV genes are detectable using as little as 0.1 pg of input DNA.

FIG. 5 is a series of digital images captured from scans of an oligonucleotide microarray, showing detection of target EBV nucleic acids in samples from B95-8 cells infected by EBV. Serial dilutions were used to prepare the indicated quantity of input sample DNA. Red spots are oligonucleotide probes representing different ORFs of the EBV genome, as listed in Table 4. Sense and antisense probe pairs were spotted in duplicate, in rows, following the order listed in the Table. Green and yellow spots are oligonucleotide probes representing human house-keeping genes and other control elements. After serial dilution of input DNA samples, some EBV genes are detectable using as little as 1 pg of input DNA.

FIG. 6 is a digital image captured from a scan of an oligonucleotide microarray, showing detection of target Ebola nucleic acids in a blood sample from a primate infected by Ebola Zaire. Red spots are oligonucleotide probes representing different ORFs of the Ebola Zaire genome, as listed in Table 6. Green and yellow spots are oligonucleotide probes representing human house-keeping genes and other control elements.

FIG. 7 is a series of digital images captured from scans of an oligonucleotide microarray, showing detection of target P. aeruginosa nucleic acids in blood samples spiked with the indicated amount of P. aeruginosa bacteria. Red spots are oligonucleotide probes representing different ORFs of the P. aeruginosa genome, as listed in Table 8. Sense and antisense probe pairs were spotted in two rows, following the order listed in the Table; also included were a pair of oligonucleotide probes representing control elements. Some P. aeruginosa genes are detectable when the sample is spiked with a single bacterium.

FIG. 8 is a series of digital images captured from scans of an oligonucleotide microarray, showing detection of target HIV-based retroviral vector nucleic acids in blood samples spiked with the indicated amount of vector material. Red spots are oligonucleotide probes representing different ORFs, as listed in Table 10. Sense and antisense probe pairs were spotted in two rows, following the order listed in the Table; also included were a pair of oligonucleotide probes representing control elements. Some HIV-based retroviral vector ORFs are detectable when the sample is spiked with a single copy of the vector.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. In the accompanying sequence listing:

SEQ ID NOs: 1-62 show the nucleic acid sequence of several representative KSHV oligonucleotide primers.

SEQ ID NOs: 63-124 show the nucleic acid sequence of several representative EBV oligonucleotide primers.

SEQ ID NOs: 125-186 show the nucleic acid sequence of several representative KSHV oligonucleotide probes.

SEQ ID NOs: 187-248 show the nucleic acid sequence of several representative EBV oligonucleotide probes.

SEQ ID NOs: 249-440 show the nucleic acid sequence of several representative pathogen-specific oligonucleotide primers.

SEQ ID NOs: 441-632 show the nucleic acid sequence of several representative pathogen-specific oligonucleotide probes.

SEQ ID NOs: 633-646 show the nucleic acid sequence of several representative Pseudomonas aeruginosa-specific oligonucleotide primers.

SEQ ID NOs: 647-660 show the nucleic acid sequence of several representative Pseudomonas aeruginosa-specific oligonucleotide probes.

SEQ ID NOs: 661-672 show the nucleic acid sequence of several representative HIV-based retroviral vector-specific oligonucleotide primers.

SEQ ID NOs: 673-684 show the nucleic acid sequence of several representative HIV-based retroviral vector-specific oligonucleotide probes.

### DETAILED DESCRIPTION

I. Abbreviations

aa: amino-allyl

BAL: bronchoalveolar lavage

cDNA: complementary DNA

DNA: deoxyribonucleic acid

EBV: Epstein-Barr virus

KSHV: Kaposi's sarcoma-associated herpesvirus

ORF: open reading frame

PCR: polymerase chain reaction

RNA: ribonucleic acid

RT-PCR: reverse transcription-polymerase chain reaction

II. Terms

Unless otherwise noted, technical terms are used according to conventional usage. Definitions of common terms in molecular biology may be found in Benjamin Lewin, Genes VII, published by Oxford University Press, 2000 (ISBN 019879276X); Kendrew et al. (eds.), The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341); and other similar references.

In order to facilitate review of the various embodiments of this disclosure, the following explanations of specific terms are provided:

Addressable: Capable of being reliably and consistently located and identified, as in an addressable location on an array.

Amplification: An increase in the amount of (number of copies of) a nucleic acid sequence, wherein the increased sequence is the same as or complementary to the existing nucleic acid template. An example of amplification is the polymerase chain reaction (PCR), in which a biological sample collected from a subject is contacted with a pair of oligonucleotide primers, under conditions that allow for the hybridization (annealing) of the primers to nucleic acid template in the sample. The primers are extended under suitable conditions (though nucleic acid polymerization). If additional copies of the nucleic acid are desired, the first copy is dissociated from the template, and additional copies of the primers (usually contained in the same reaction mixture) are annealed to the template, extended, and dissociated repeatedly to amplify the desired number of copies of the nucleic acid.

The products of amplification may be characterized by, for instance, electrophoresis, restriction endonuclease cleavage patterns, hybridization, ligation, and/or nucleic acid sequencing, using standard techniques.

Other examples of in vitro amplification techniques include reverse-transcription PCR (RT-PCR), strand displacement amplification (see U.S. Pat. No. 5,744,311); transcription-free isothermal amplification (see U.S. Pat. No. 6,033,881); repair chain reaction amplification (see WO 90/01069); ligase chain reaction amplification (see EP-A-320 308); gap filling ligase chain reaction amplification (see U.S. Pat. No. 5,427,930); coupled ligase detection and PCR (see U.S. Pat. No. 6,027,889); and NASBA™ RNA transcription-free amplification (see U.S. Pat. No. 6,025,134).

Animal: Living multi-cellular vertebrate organisms, a category that includes, for example, mammals and birds. The term mammal includes both human and non-human mammals. Similarly, the term “subject” includes both human and veterinary subjects, for example, humans, non-human primates, dogs, cats, horses, and cows.

Antisense and sense: Double-stranded DNA (dsDNA) has two strands, a 5′ to 3′ strand, referred to as the plus strand, and a 3′ to 5′ strand, referred to as the minus strand. Because RNA polymerase adds nucleic acids in a 5′ to 3+ direction, the minus strand of the DNA serves as the template for the RNA during transcription. Thus, the RNA formed will have a sequence complementary to the minus strand, and identical to the plus strand (except that the base uracil is substituted for thymine).

Antisense molecules are molecules that are specifically hybridizable or specifically complementary to either RNA or the plus strand of DNA. Sense molecules are molecules that are specifically hybridizable or specifically complementary to the minus strand of DNA.

Array: An arrangement of molecules, particularly biological macromolecules (such as nucleic acids), in addressable locations on a solid support. The individual molecules placed on the array are termed “probes.” The array may be regular (arranged in uniform rows and columns, for instance) or irregular. The number of addressable locations on the array can vary, for example from a few (such as three) to more than 50, 100, 200, 500, 1000, 10,000, or more. A “microarray” is an array that is miniaturized so as to require microscopic examination for evaluation.

Within an array, each arrayed probe is addressable, in that its location can be reliably and consistently determined within the at least two dimensions of the array surface. In ordered arrays the location of each probe sample can be assigned to the sample at the time when it is spotted onto the array surface, and a key may be provided in order to correlate each location with the appropriate target. Often, ordered arrays are arranged in a symmetrical grid pattern, but samples could be arranged in other patterns (e.g., in radially distributed lines, spiral lines, or ordered clusters). Addressable arrays are computer readable, in that a computer can be programmed to correlate a particular address on the array with information (such as hybridization or binding data, including for instance signal intensity). In some examples of computer readable formats, the individual “spots” on the array surface will be arranged regularly in a pattern (e.g., a Cartesian grid pattern) that can be correlated to address information by a computer.

The sample application “feature” or “spot” on an array may assume many different shapes. Thus, though the term “feature” or “spot” is used, it refers generally to a localized deposit of nucleic acid, and is not limited to a round or substantially round region. For instance, substantially square regions of mixture application can be used with arrays encompassed herein, as can be regions that are substantially rectangular (such as a slot blot-type application), or triangular, oval, or irregular. The shape of the array support itself is also immaterial, though it is usually substantially flat and may be rectangular or square in general shape.

Binding or interaction: An association between two substances or molecules, such as the hybridization of one nucleic acid molecule to another (or itself). The disclosed oligonucleotide arrays are used to detect binding of an amplified target nucleic acid sequence (the target) to an immobilized nucleic acid molecule (the probe) in one or more features of the array. A target “binds” to an immobilized probe in a feature on an array if, after incubation of the (labeled) target (usually in solution or suspension) with or on the array for a period of time (usually 5 minutes or more, for instance 10 minutes, 20 minutes, 30 minutes, 60 minutes, 90 minutes, 120 minutes or more, for instance over night or even 24 hours), a detectable amount of that labeled target associates with a probe of the array to such an extent that it is not removed by being washed with a relatively low stringency buffer (e.g., higher salt, such as 3×SSC or higher, and room temperature washes). Washing can be carried out, for instance, at room temperature, but other temperatures (either higher or lower) also can be used. Targets will bind probe nucleic acid molecules within different features on the array to different extents, based at least on sequence homology, and the term “bind” encompasses both relatively weak and relatively strong interactions. Thus, some binding will persist after the array is washed in a more stringent buffer (e.g., lower salt, such as about 0.5 to about 1.5×SSC, and 55-65° C. washes).

Where the two substances or molecules are both nucleic acids, binding of the target to a probe nucleic acid molecule on the array can be discussed in terms of the specific complementarity between the probe and the target nucleic acids.

cDNA (complementary DNA): A piece of DNA lacking internal, non-coding segments (introns) and transcriptional regulatory sequences. cDNA can also contain untranslated regions (UTRs) that are responsible for translational control in the corresponding RNA molecule. cDNA is usually synthesized in the laboratory by reverse transcription from messenger RNA extracted from cells.

DNA (deoxyribonucleic acid): A long chain polymer which comprises the genetic material of most living organisms (some viruses have genes comprising ribonucleic acid (RNA)). The repeating units in DNA polymers are four different nucleotides, each of which comprises one of the four bases, adenine, guanine, cytosine and thymine bound to a deoxyribose sugar to which a phosphate group is attached. Triplets of nucleotides (referred to as codons) code for each amino acid in a polypeptide. The term codon is also used for the corresponding (and complementary) sequences of three nucleotides in the mRNA into which the DNA sequence is transcribed.

Unless otherwise specified, any reference to a DNA molecule is intended to include the reverse complement of that DNA molecule. Except where single-strandedness is required by the text herein, DNA molecules, though written to depict only a single strand, encompass both strands of a double-stranded DNA molecule. Thus, a reference to the nucleic acid molecule that encodes a specific protein, or a fragment thereof, encompasses both the sense strand and its reverse complement. For instance, it is contemplated that probes or primers can be generated from the reverse complement sequence of the disclosed nucleic acid molecules.

Fluorophore: A chemical compound, which when excited by exposure to a particular wavelength of light, emits light (i.e., fluoresces), for example at a different wavelength than that to which it was exposed. Fluorophores can be described in terms of their emission profile, or “color.” Green fluorophores, for example Cy3, FITC, and Oregon Green, are characterized by their emission at wavelengths generally in the range of 515-540 λ. Red fluorophores, for example Texas Red, Cy5 and tetramethylrhodamine, are characterized by their emission at wavelengths generally in the range of 590-690 λ.

Encompassed by the term “fluorophore” as it is used herein are luminescent molecules, which are chemical compounds which do not require exposure to a particular wavelength of light to fluoresce; luminescent compounds naturally fluoresce. Therefore, the use of luminescent signals eliminates the need for an external source of electromagnetic radiation, such as a laser. An example of a luminescent molecule includes, but is not limited to, aequorin (Tsien, Ann. Rev. Biochem. 67:509-44, 1998).

Examples of fluorophores are provided in U.S. Pat. No.5,866,366. These include: 4-acetamido-4′-isothiocyanatostilbene-2,2′disulfonic acid, acridine and derivatives such as acridine and acridine isothiocyanate, 5-(2′-aminoethyl)aminonaphthalene-1-sulfonic acid (EDANS), 4-amino-N-[3-vinylsulfonyl)phenyl]naphthalimide-3,5 disulfonate (Lucifer Yellow VS), N-(4-anilino-1-naphthyl)maleimide, anthranilamide, Brilliant Yellow, coumarin and derivatives such as coumarin, 7-amino-4-methylcoumarin (AMC, Coumarin 120), 7-amino-4-trifluoromethylcouluarin (Coumaran 151); cyanosine; 4′,6-diaminidino-2-phenylindole (DAPI); 5′,5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6-dichlorotriazin-2-yl)aminofluorescein (DTAF), 2′7′-dimethoxy-4′5′-dichloro-6-carboxyfluorescein (JOE), fluorescein, fluorescein isothiocyanate (FITC), and QFITC (XRITC); fluorescamine; IR144; IR1446; Malachite Green isothiocyanate; 4-methylumbelliferone; ortho cresolphthalein; nitrotyrosine; pararosaniline; Phenol Red; B-phycoerythrin; o-phthaldialdehyde; pyrene and derivatives such as pyrene, pyrene butyrate and succinimidyl 1-pyrene butyrate; Reactive Red 4 (Cibacron .RTM. Brilliant Red 3B-A); rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, sulforhodamine B, sulforhodamine 101 and sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid and terbium chelate derivatives.

Other fluorophores include thiol-reactive europium chelates that emit at approximately 617 nm (Heyduk and Heyduk, Analyt. Biochem. 248:216-27, 1997; J. Biol. Chem. 274:3315-22, 1999).

Additional fluorophores include cyanine, merocyanine, styryl, and oxonyl compounds, such as those disclosed in U.S. Pat. Nos. 5,268,486; 5,486,616; 5,627,027; 5,569,587; and 5,569,766, and in published PCT patent application no. US98/00475. Specific examples of fluorophores disclosed in one or more of these patent documents include Cy3 and Cy5, for instance.

Still other fluorophores include GFP, Lissamine™, diethylaminocoumarin, fluorescein chlorotriazinyl, naphthofluorescein, 4,7-dichlororhodamine and xanthene (as described in U.S. Pat. No. 5,800,996) and derivatives thereof. Other fluorophores are known to those skilled in the art, for example those available from Molecular Probes (Eugene, Oreg.).

Particularly useful fluorophores have the ability to be attached to (coupled with) a nucleotide, such as a modified nucleotide, are substantially stable against photobleaching, and have high quantum efficiency.

Hybridization: Oligonucleotides and their analogs hybridize by hydrogen bonding, which includes Watson-Crick, Hoogsteen or reversed Hoogsteen hydrogen bonding, between complementary bases. Generally, nucleic acid consists of nitrogenous bases that are either pyrimidines (cytosine (C), uracil (U), and thyrnine (T)) or purines (adenine (A) and guanine (G)). These nitrogenous bases form hydrogen bonds between a pyrimidine and a purine, and the bonding of the pyrimidine to the purine is referred to as “base pairing.” More specifically, A will hydrogen bond to T or U, and G will bond to C. “Complementary” refers to the base pairing that occurs between to distinct nucleic acid sequences or two distinct regions of the same nucleic acid sequence.

“Specifically hybridizable,” “specifically hybridizes” and “specifically complementary” are terms which indicate a sufficient degree of complementarity such that stable and specific binding occurs between an oligonucleotide and its DNA or RNA target. An oligonucleotide need not be 100% complementary to its target DNA or RNA sequence to be specifically hybridizable. An oligonucleotide is specifically hybridizable when binding of the oligonucleotide to the target DNA or RNA molecule interferes with the normal function of the target DNA or RNA, and there is a sufficient degree of complementarity to avoid non-specific binding of the oligonucleotide to non-target sequences under conditions in which specific binding is desired, or under conditions in which an assay is performed.

Hybridization conditions resulting in particular degrees of stringency will vary depending upon the nature of the hybridization method of choice and the composition and length of the hybridizing nucleic acid sequences. Generally, the temperature of hybridization and the ionic strength (especially the Na⁺ and/or Mg⁺⁺ concentration) of the hybridization buffer will determine the stringency of hybridization, though wash times also influence stringency. Calculations regarding hybridization conditions required for attaining particular degrees of stringency are discussed by Sambrook et al. (ed.), Molecular Cloning: A Laboratory Manual, 2^{nd} ed., vol. 1-3, Cold Spring Harbor Laboratory Press, Cold Spring Harbor, N.Y., 1989, chapters 9 and 11; and Ausubel et al. Short Protocols in Molecular Biology, 4^{th} ed., John Wiley & Sons, Inc., 1999.

Isolated/purified: An “isolated” or “purified” biological component (such as a nucleic acid, peptide or protein) has been substantially separated, produced apart from, or purified away from other biological components in the cell of the organism in which the component naturally occurs, that is, other chromosomal and extrachromosomal DNA and RNA, proteins, lipids, and so forth. Nucleic acids, peptides and proteins that have been “isolated” thus include nucleic acids and proteins purified by standard purification methods. The term also embraces nucleic acids, peptides and proteins prepared by recombinant expression in a host cell as well as chemically synthesized nucleic acids or proteins.

The terms “isolated” and “purified” do not require absolute purity; rather, it is intended as a relative term. Thus, for example, an isolated biological component is one in which the biological component is more enriched than the biological component is in its natural environment within a cell. Preferably, a preparation is isolated or purified such that the biological component represents at least 50%, such as at least 70%, at least 90%, at least 95%, or greater of the total biological component content of the preparation.

Label: A detectable compound or composition that is conjugated or otherwise attached directly or indirectly to another molecule to facilitate detection of that molecule. Specific, non-limiting examples of labels include radioactive isotopes, enzyme substrates, co-factors, ligands, chemiluminescent or fluorescent markers or dyes, haptens, and enzymes. Methods for labeling and guidance in the choice of labels appropriate for various purposes are discussed, for example, in Sambrook et al. (ed.), Molecular Cloning: A Laboratory Manual, 2^{nd} ed., vol. 1-3, Cold Spring Harbor Laboratory Press, Cold Spring Harbor, N.Y., 1989 and Ausubel et al. Short Protocols in Molecular Biology, 4^{th} ed., John Wiley & Sons, Inc., 1999.

Microorganism: A microscopic organism, a category that includes, for example, bacteria, viruses, protozoa, and some plants and animals.

Modified nucleotide: A modified nucleotide is a nucleotide to which a chemical moiety has been added, usually one that gives an additional functionality to the modified nucleotide. Generally, the modification comprises a functional group or a leaving group, and permits coupling of the nucleotide to a detectable molecule, such as a fluorophore or hapten.

For instance, one specific class of modifications are those that add a reactive amine group to the nucleotide; an amino-allyl group is one such amine modification. Amine groups are reactive with a wide spectrum of other chemical groups, which will be known to one of ordinary skill in the art. By way of example, amine groups are reactive with intermediate N-hydroxysuccinimide (NHS) esters, such as those on NHS ester cyanine dyes. Amine groups also can be reacted with peptide molecules (such as antigenic fragments or antibody or antibody fragment) or biotin (for instance, to which a fluorescent dye can then be coupled), for instance. Examples of amine-reactive fluorophores that can be coupled to amine modified-nucleotides include, but are not limited to, fluorescein, BODIPY, rhodamine, Texas Red, cyanine dyes, and their derivatives. Reaction of amine-reactive fluorophores usually proceeds at pH values in the range of pH 7-10.

Alternatively, thiol-reactive fluorophores can be used to generate a fluorescently-labeled nucleotide or oligonucleotide. Thus, also contemplated herein are nucleotides (and oligonucleotides) containing a thiol group as its modification. Reaction of fluors with thiols usually proceeds rapidly at or below room temperature (RT) in the physiological pH range (pH 6.5-8.0) to yield chemically stable thioesters. Examples of thiol-reactive fluorophores include, but are not limited to: fluorescein, BODIPY, cumarin, rhodamine, Texas Red and their derivatives.

Other functional groups that can be added to a nucleotide to make a modified nucleotide include alcohols and carboxylic acids. These reactive functional groups also can be used to couple a fluorophore to the nucleotide or oligonucleotide.

In particular embodiments, fluorescently-labeled nucleotides/oligonucleotides have a high fluorescence yield, and retain the critical features of the nucleotide/oligonucleotide, primarily the ability to bind to a complementary strand of a nucleic acid molecule and prime a polymerizing reaction. The term also includes nucleotides containing modified bases, modified sugar moieties and modified phosphate backbones, for example as described in U.S. Pat. No. 5,866,336.

Examples of modified base moieties which can be used to modify nucleotides at any position on its structure include, but are not limited to: 5-fluorouracil, 5-bromouracil, 5-chlorouracil, 5-iodouracil, hypoxanthine, xanthine, acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N-6-sopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid, pseudouracil, queosine, 2-thiocytosine, 5-methyl-2-thiouracil, 2-thiouracil, 4-thiouracil, 5-methyluracil, uracil-5-oxyacetic acid methylester, uracil-S-oxyacetic acid, 5-methyl-2-thiouracil, 3-(3-amino-3-N-2-carboxypropyl)uracil, and 2,6-diaminopurine.

Examples of modified sugar moieties which may be used to modify nucleotides at any position on its structure include, but are not limited to: arabinose, 2-fluoroarabinose, xylose, and hexose, or a modified component of the phosphate backbone, such as phosphorothioate, a phosphorodithioate, a phosphoramidothioate, a phosphoramidate, a phosphordiamidate, a methylphosphonate, an alkyl phosphotriester, or a formacetal or analog thereof.

Also included in the term “modified nucleotide” are branched nucleotides bearing more than one modification. Examples of branched nucleotides are disclosed, for instance, in Horn and Urdea (Nuc. Acids Res. 17:6959-67, 1989) and Nelson et al. (Nuc. Acids Res. 17:7179-86, 1989).

In certain embodiments, modifications to nucleotides allow for incorporation of the nucleotide into a growing nucleic acid chain, for instance through in vitro chemical synthesis (e.g., by phosphoramidite synthesis).

Multiplex PCR: Polymerase chain reaction in a single reaction tube that uses multiplex primers to produce more than one PCR product that can be detected.

Multiplex primers: More than one pair of primers that are used simultaneously to amplify more than one target and/or control nucleic acid molecule in a single reaction tube.

Nucleic acid sequence (or polynucleotide): A deoxyribonucleotide or ribonucleotide polymer in either single or double stranded form, and unless otherwise limited, encompasses known analogues of natural nucleotides that hybridize to nucleic acids in a manner similar to naturally occurring nucleotides, and includes polynucleotides encoding full length proteins and/or fragments of such full length proteins which can function as a therapeutic agent. A polynucleotide is generally a linear nucleotide sequence, including sequences of greater than 100 nucleotide bases in length. In one embodiment, a nucleic acid is labeled (for example, biotinylated, fluorescently labeled or radiolabled nucleotides).

Nucleotide: “Nucleotide” includes, but is not limited to, a monomer that includes a base linked to a sugar, such as a pyrimidine, purine or synthetic analogs thereof, or a base linked to an amino acid, as in a peptide nucleic acid (PNA). A nucleotide is one monomer in an oligonucleotide/polynucleotide. A nucleotide sequence refers to the sequence of bases in an oligonucleotide/polynucleotide.

The major nucleotides of DNA are deoxyadenosine 5′-triphosphate (dATP or A), deoxyguanosine 5′-triphosphate (dGTP or G), deoxycytidine 5′-triphosphate (dCTP or C) and deoxythymidine 5′-triphosphate (dTTP or T). The major nucleotides of RNA are adenosine 5′-triphosphate (ATP or A), guanosine 5′-triphosphate (GTP or G), cytidine 5′-triphosphate (CTP or C) and uridine 5′-triphosphate (UTP or U). Inosine is also a base that can be integrated into DNA or RNA in a nucleotide (dITP or ITP, respectively).

Oligonucleotide: A nucleic acid molecule generally comprising a length of 300 bases or fewer. The term often refers to single-stranded deoxyribonucleotides, but it can refer as well to single- or double-stranded ribonucleotides, RNA:DNA hybrids and double-stranded DNAs, among others. The term “oligonucleotide” also includes oligonucleosides, that is, an oligonucleotide minus the phosphate. In some examples, oligonucleotides are about 10 to about 90 bases in length, for example, 12, 13, 14, 15, 16, 17, 18, 19 or 20 bases in length. Other oligonucleotides are about 25, about 30, about 35, about 40, about 45, about 50, about 55, about 60 bases, about 65 bases, about 70 bases, about 75 bases or about 80 bases in length.

Oligonucleotides may be single-stranded, for example, for use as probes or primers, or may be double-stranded, for example, for use in the construction of a mutant gene. Oligonucleotides can be either sense or antisense oligonucleotides. An oligonucleotide can be modified as discussed herein in reference to nucleic acid molecules. Oligonucleotides can be obtained from existing nucleic acid sources (for example, genomic or cDNA), but can also be synthetic (for example, produced by laboratory or in vitro oligonucleotide synthesis).

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Pathogen: Any disease producing microorganism, a category that includes, for example: Bacillus anthracis, Clostridium botulinum, Brucella species, Burkholderia mallei, Burkholderia pseudomallei, Chlamydia psittaci, Vibrio cholerae, Clostridium perfringens, Coxiella burnetii, Escherichia coli (e.g., E. coli 0157:H7), Nipah Virus, Salmonella species, Shigella, Francisella tularensis, Yersinia pestis, Rickettsia prowazekii, Salmonella typhi, Variola major, Alphaviruses e.g., Venezuelan Equine Encephalitis, Eastern Equine Encephalitis and Western Equine Encephalitis), Bunyaviruses (e.g., Hantavirus, Rift Valley Fever and Crimean-Congo Hemorrhagic Fever), Flaviviruses (e.g., Dengue), Filoviruses (e.g., Ebola and Marburg), Arenaviruses (e.g., Guanarito, Junin, Lassa Fever, Lymphocytic Choriomeningitis Virus, and Machupo), SARS-associated coronavirus (SARS-CoV), and Cryptosporidium parvum.

Polymerase chain reaction (PCR): A method for amplifying specific DNA segments which exploits certain features of DNA replication. For instance replication requires a primer, and specificity is determined by the sequence and size of the primer. One primer is complementary to the sense-strand at one end of the DNA sequence to be amplified and the other primer is complementary to the antisense-strand at the other end of the DNA sequence to be amplified. The PCR amplifies specific DNA segments by cycles of template denaturation; primer addition; primer annealing; and replication using a thermostable DNA polymerase. Because the newly synthesized DNA strands subsequently serve as additional templates for the same primer sequences, successive rounds of primer annealing, strand elongation and dissociation produce rapid and highly specific amplification of the desired sequence. PCR can be used, for instance, to detect a defined target sequence in a DNA sample.

Polymerization: Synthesis of a new nucleic acid chain (oligonucleotide or polynucleotide) by adding nucleotides to the hydroxyl group at the 3′-end of a pre-existing RNA or DNA primer using a pre-existing DNA strand as the template. Polymerization usually is mediated by an enzyme such as a DNA or RNA polymerase. Specific examples of polymerases include the large proteolytic fragment of the DNA polymerase I of the bacterium E. coli (usually referred to as Klenow polymerase), E. coli DNA polymerase I, and bacteriophage T7 DNA polymerase. Polymerization of a DNA strand complementary to an RNA template (e.g., a cDNA complementary to a mRNA) can be carried out using reverse transcriptase (in a reverse transcription reaction).

For in vitro polymerization reactions, it is necessary to provide to the assay mixture an amount of required cofactors such as M⁺⁺, and dATP, dCTP, dGTP, dTTP, ATP, CTP, GTP, UTP or other nucleoside triphosphates, in sufficient quantity to support the degree of amplification desired. The amounts of deoxyribonucleotide triphosphates substrates required for polymerizing reactions are well known to those of ordinary skill in the art. Nucleoside triphosphate analogues or modified nucleoside triphosphates can be substituted or added to those specified above.

Polypeptide: A polymer in which the monomers are amino acid residues which are joined together through amide bonds. When the amino acids are alpha-amino acids, either the L-optical isomer or the D-optical isomer can be used. The terms “polypeptide” or “protein” as used herein are intended to encompass any amino acid sequence and include modified sequences such as glycoproteins. The term “polypeptide” is specifically intended to cover naturally occurring proteins, as well as those which are recombinantly or synthetically produced. The term “residue” or “amino acid residue” includes reference to an amino acid that is incorporated into a peptide, polypeptide, or protein.

Conservative amino acid substitutions are those substitutions that, when made, least interfere with the properties of the original protein, that is, the structure and especially the function of the protein is conserved and not significantly changed by such substitutions. Examples of conservative substitutions are shown below:

| Original | Conservative |
|---|---|
| Residue | Substitutions |
| Ala | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Conservative substitutions generally maintain (a) the structure of the polypeptide backbone in the area of the substitution, for example, as a sheet or helical conformation, (b) the charge or hydrophobicity of the molecule at the target site, or (c) the bulk of the side chain.

The substitutions which in general are expected to produce the greatest changes in protein properties will be non-conservative, for instance changes in which (a) a hydrophilic residue, for example, seryl or threonyl, is substituted for (or by) a hydrophobic residue, for example, leucyl, isoleucyl, phenylalanyl, valyl or alanyl; (b) a cysteine or proline is substituted for (or by) any other residue; (c) a residue having an electropositive side chain, for example, lysyl, arginyl, or histadyl, is substituted for (or by) an electronegative residue, for example, glutamyl or aspartyl; or (d) a residue having a bulky side chain, for example, phenylalanine, is substituted for (or by) one not having a side chain, for example, glycine.

Probes and primers: As used herein, a probe comprises an isolated nucleic acid molecule, optionally attached to a solid support. Probes are relatively short nucleic acid molecules, for instance DNA oligonucleotides 10 nucleotides or more in length, such as about 15, 25, 35, 55, 75, or 95 nucleotides or more in length. Probes can be annealed to complementary amplified target nucleic acids by nucleic acid hybridization to form hybrids between the probe and the amplified target nucleic acids.

Primers are relatively short nucleic acid molecules, for instance DNA oligonucleotides 10 nucleotides or more in length, for example that hybridize to contiguous complementary nucleotides or a sequence to be amplified. Longer DNA oligonucleotides may be about 15, 20, 25, 30, 50, 75, or 90 nucleotides or more in length. Primers can be annealed to a complementary target DNA strand by nucleic acid hybridization to form a hybrid between the primer and the target DNA strand, and then the primer extended along the target DNA strand by a DNA polymerase enzyme. Primer pairs can be used for amplification of a nucleic acid sequence, for example, by the PCR or other nucleic acid amplification methods known in the art, as described above.

Methods for preparing and using nucleic acid probes and primers are described, for example, in Sambrook et al. (ed.), Molecular Cloning: A Laboratory Manual, 2^{nd} ed., vol. 1-3, Cold Spring Harbor Laboratory Press, Cold Spring Harbor, N.Y., 1989; Ausubel et al. Short Protocols in Molecular Biology, 4^{th} ed., John Wiley & Sons, Inc., 1999; and Innis et al. PCR Protocols, A Guide to Methods and Applications, Academic Press, Inc., San Diego, Calif., 1990. Amplification primer pairs can be derived from a known sequence, for example, by using computer programs intended for that purpose such as Primer (Version 0.5, © 1991, Whitehead Institute for Biomedical Research, Cambridge, Mass.). One of ordinary skill in the art will appreciate that the specificity of a particular probe or primer increases with its length. Thus, in order to obtain greater specificity, probes and primers can be selected that comprise at least 20, 25, 30, 35, 40, 45, 50, 75, 90 or more consecutive nucleotides of a target nucleotide sequence.

Recombinant: A recombinant nucleic acid is one that has a sequence that is not naturally occurring or has a sequence that is made by an artificial combination of two otherwise separated segments of sequence. This artificial combination can be accomplished by chemical synthesis or, more commonly, by the artificial manipulation of isolated segments of nucleic acids, for example, by genetic engineering techniques.

RNA: A typically linear polymer of ribonucleic acid monomers, linked by phosphodiester bonds. Naturally occurring RNA molecules fall into three classes, messenger (mRNA, which encodes proteins), ribosomal (rRNA, components of ribosomes), and transfer (tRNA, molecules responsible for transferring amino acid monomers to the ribosome during protein synthesis). Total RNA refers to a heterogeneous mixture of all three types of RNA molecules.

Sample: A portion, piece, or segment that is representative of the whole from which the sample is obtained. This term encompasses any material, including for instance samples obtained from an animal, a plant, or the environment.

An “environmental sample” includes a sample obtained from inanimate objects or reservoirs within an indoor or outdoor environment. Environmental samples include, but are not limited to: soil, water, dust, and air samples; bulk samples, including building materials, furniture, and landfill contents; and other reservoir samples, such as animal refuse, harvested grains, and foodstuffs. It is to be understood that environmental samples can and often do contain biological components.

A “biological sample” is a sample obtained from a plant or animal. As used herein, biological samples include all samples useful for detection of pathogen infection in subjects, including, but not limited to: cells, tissues, and bodily fluids, such as blood; derivatives and fractions of blood (such as serum); extracted galls; biopsied or surgically removed tissue, including tissues that are, for example, unfixed, frozen, fixed in formalin and/or embedded in paraffin; tears; milk; skin scrapes; surface washings; urine; sputum; cerebrospinal fluid; prostate fluid; semen; pus; bone marrow aspirates; bronchoalveolar lavage (BAL); saliva; cervical swabs; vaginal swabs; and oropharyngeal wash.

Sequence identity: The similarity between two nucleic acid sequences, or two amino acid sequences, is expressed in terms of the similarity between the sequences, otherwise referred to as sequence identity. Sequence identity is frequently measured in terms of percentage identity (or similarity or homology); the higher the percentage, the more similar the two sequences are.

Methods of alignment of sequences for comparison are well known in the art. Various programs and alignment algorithms are described in: Smith and Waterman (Adv. Appl. Math., 2:482, 1981); Needleman and Wunsch (J. Mol. Biol., 48:443, 1970); Pearson and Lipman (Proc. Natl. Acad. Sci., 85:2444, 1988); Higgins and Sharp (Gene, 73:237-44, 1988); Higgins and Sharp (CABIOS, 5:151-53, 1989); Corpet et al. (Nuc. Acids Res., 16:10881-90, 1988); Huang et al. (Comp. Appls Biosci., 8:155-65, 1992); and Pearson et al. (Meth. Mol. Biol., 24:307-31, 1994). Altschul et al. (Nature Genet., 6:119-29, 1994) presents a detailed consideration of sequence alignment methods and homology calculations.

The alignment tools ALIGN (Myers and Miller, CABIOS 4:11-17, 1989) or LFASTA (Pearson and Lipman, 1988) may be used to perform sequence comparisons (Internet Program © 1996, W. R. Pearson and the University of Virginia, “fasta20u63” version 2.0u63, release date December 1996). ALIGN compares entire sequences against one another, while LFASTA compares regions of local similarity. These alignment tools and their respective tutorials are available on the Internet at the National Center for Supercomputing Applications website.

Alternatively, the NCBI Basic Local Alignment Search Tool (BLAST) (Altschul et al., J. Mol. Biol., 215:403-10, 1990; Gish. and States, Nature Genet., 3:266-72, 1993; Madden et al., Meth. Enzymol., 266:131-41, 1996; Altschul et al., Nucleic Acids Res., 25:3389-402, 1997; and Zhang and Madden, Genome Res., 7:649-56, 1997) is available from several sources, including the National Center for Biotechnology Information (NCBI, Bethesda, Md.) and on the Internet, for use in connection with the sequence analysis programs blastp, blastn, blastx, tblastn, and tblastx. It can be accessed through the NCBI website. A description of how to determine sequence identity using this program also is available on the NCBI website; usually, default settings will be used for most comparisons.

An alternative indication that two nucleic acid molecules are closely related is that the two molecules hybridize to each other under stringent conditions. Stringent conditions are sequence-dependent and are different under different environmental parameters. Generally, stringent conditions are selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Conditions for nucleic acid hybridization and calculation of stringencies can be found, for example, in Sambrook et al., Molecular Cloning: A Laboratory Manual, Cold Spring Harbor Laboratory Press, Cold Spring Harbor, N.Y., 2001 and Tijssen, Hybridization With Nucleic Acid Probes, Part I: Theory and Nucleic Acid Preparation, Laboratory Techniques in Biochemistry and Molecular Biology, Elsevier Science Ltd., NY, N.Y., 1993.

For purposes of the present disclosure, “stringent conditions” encompass conditions under which hybridization will only occur if there is less than 25% mismatch between the hybridization molecule and the target sequence. “Stringent conditions” may be broken down into particular levels of stringency for more precise definition. Thus, as used herein, “moderate stringency” conditions are those under which molecules with more than 25% sequence mismatch will not hybridize; conditions of “medium stringency” are those under which molecules with more than 15% mismatch will not hybridize, and conditions of “high stringency” are those under which sequences with more than 10% mismatch will not hybridize. Conditions of “very high stringency” are those under which sequences with more than 6% mismatch will not hybridize.

Solid support: “Solid support” includes, but is not limited to, glass, silicon, metals (e.g., gold), polymer films (e.g., polymers having a substantially non-porous surface); polymer filaments (e.g., mesh and fabrics); polymer beads; polymer foams; polymer frits; and polymer threads. Polymers can include, but are not limited to, cellulosic substrates, such as nitrocellulose, nylon, TEFLON , polypropylene, polyethylene, polybutylene, polyisobutylene, polybutadiene, polyisoprene, polyvinylpyrrolidine, polytetrafluroethylene, polyvinylidene difluoride, polyfluoroethylene-propylene, polyethylenevinyl alcohol, polymethylpentene, polycholorotrifluoroethylene, polysulfomes, biaxially oriented polypropylene (BOPP), hydroxylated BOPP, aminated BOPP, thiolated BOPP, etyleneacrylic acid, thylene methacrylic acid, and blends of copolymers thereof.

Stripping: Bound target molecules can be stripped from an array, for instance a cDNA array, in order to use the same array for another probe interaction analysis (e.g., to determine gene expression level in a different cell sample or determine sensitivity using a different amount of input DNA). Any process that will remove substantially all of the prior target molecule from the array, without also significantly removing the immobilized nucleic acid probes of the array, can be used. By way of example only, one method for stripping an array is by boiling it in stripping buffer (e.g., very low or no salt with 0.1% SDS), for instance for about an hour or more. The stripped array may be washed, for instance in an equilibrating or low stringency buffer, prior to incubation with another target molecule.

Where a stripability enhancer (such as the nucleotide analog of the STRIPABLE™ and STRIP-EZ™ system from Ambion (Austin, Tex.)) is used, the procedures provided by the manufacturer for use with this product provide a good starting point for tailoring probing and stripping conditions for use with arrays. Addition of stripability enhancers to probes for use with arrays is optional.

Target nucleic acid sequence: A nucleic acid sequence to which an antisense or sense oligonucleotide primer or probe specifically hybridizes. A target nucleic acid sequence can be amplified, for example, by using a pair of primers complementary to the target nucleic acid sequence and a DNA polymerase enzyme in a PCR or other nucleic acid amplification method known to one of skill in the art.

Optionally, a detectable label or other reporter molecule can be attached to the amplified target nucleic acid. Typical labels include radioactive isotopes, enzyme substrates, co-factors, ligands, chemiluminescent or fluorescent markers or dyes, haptens, and enzymes. Methods for labeling and guidance in the choice of labels appropriate for various purposes are discussed, for example, in Sambrook et al. (ed.), Molecular Cloning: A Laboratory Manual, 2^{nd} ed., vol. 1-3, Cold Spring Harbor Laboratory Press, Cold Spring Harbor, N.Y., 1989 and Ausubel et al. Short Protocols in Molecular Biology, 4^{th} ed., John Wiley & Sons, Inc., 1999.

Template: A nucleic acid polymer that can serve as a substrate for the synthesis of a complementary nucleic acid strand. A template nucleic acid molecule may be either DNA or RNA. Nucleic acid templates may be in a double-stranded or single-stranded form. If the nucleic acid is double-stranded at the start of the polymerization reaction, it may be treated to denature the two strands into a single-stranded, or partially single-stranded, form. Methods are known to render double-stranded nucleic acids into single-stranded, or partially single-stranded, forms, such as by heating to about 90°-100° C. for about 1 to 10 minutes, or by alkali treatment, such as treatment at a pH of 12 or greater.

Variant oligonucleotides and variant analogs: A variant of an oligonucleotide or an oligonucleotide analog is a nucleic acid oligomer having one or more base substitutions, one or more base deletions, and/or one or more base insertions, so long as the oligomer substantially retains the activity of the original oligonucleotide or analog, or has sufficient complementarity to a target sequence.

A variant oligonucleotide or analog may also hybridize with the target DNA or RNA, under stringency conditions as described above. A variant oligonucleotide or analog also exhibits sufficient complementarity with the target DNA or RNA of the original oligonucleotide or analog as described herein.

As used herein, the singular terms “a,” “an,” and “the” include plural referents unless context clearly indicates otherwise. Similarly, the word “or” is intended to include “and” unless the context clearly indicates otherwise. Also, as used herein, the term “comprises” means “includes.” Hence “comprising A or B” means including A, B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

III. Overview of Several Embodiments

Provided herein in various embodiments are oligonucleotide arrays that are useful for the qualitative detection of multiple target nucleic acids (including rare targets) in a sample. In one embodiment, an oligonucleotide array comprises a plurality of single-stranded nucleic acid probe pairs affixed at discrete addressable locations on a solid support, wherein each of the probe pairs includes an antisense nucleic acid probe sequence specifically complementary to the sense strand of a double-stranded target nucleic acid, and a sense nucleic acid probe sequence specifically complementary to the antisense strand of the double-stranded target nucleic acid. In another embodiment, each antisense nucleic acid probe sequence of a probe pair consists essentially of the complement of the corresponding sense nucleic acid probe sequence. In a specific example of the provided arrays, each antisense nucleic acid probe sequence of a probe pair consists essentially of at least one of the sequences shown in SEQ ID NOs: 429-519 and each sense nucleic acid probe sequence of the probe pair consists essentially of at least one of the sequences shown in SEQ ID NOs: 520-609.

In yet another embodiment, the number of locations on the array is from about 50 to about 1,000. In a further embodiment the solid support is flexible; specific, non-limiting examples include nylon. In yet a further embodiment, the solid support is rigid; specific, non-limiting examples include glass.

Also provided herein in various embodiments are methods that are useful for detecting target nucleic acids in a sample. In one embodiment, the method comprises extracting total nucleic acid from the sample, hybridizing a plurality of target-specific primers to the total nucleic acid, amplifying target-specific nucleic acids from the total nucleic acid utilizing the target-specific primers to produce amplified target-specific nucleic acid molecules, contacting the amplified target-specific nucleic acid molecules with an oligonucleotide array as described herein under conditions sufficient to produce a hybridization pattern, detecting the hybridization pattern, and identifying the target nucleic acids in the sample based on the hybridization pattern. In another embodiment, the method further comprises reverse transcribing a plurality of target-specific cDNAs complementary with target transcripts contained in the total nucleic acid prior to amplifying target-specific DNAs and cDNAs.

In a specific example of the provided method, the target nucleic acids comprise one or more nucleic acids from one or more pathogens, such as Bacillus anthracis, Clostridium botulinum, Yersinia pestis, Variola major, Francisella tularensis, a Filovirus, an Arenavirus, or combinations of two or more thereof. In a further specific example of the provided method, the plurality of target-specific primers are selected from the group listed in Table 5.

In another specific example of the provided method, the amplification utilizes polymerase chain reaction. In yet another specific example of the provided method, the amplified targets are labeled targets, such as an amino-allyl dNTP. In a further specific example of the provided method, a detectable label is conjugated to the amino-allyl dNTP prior to hybridizing the amplified target-specific nucleic acid molecules to the array. Specific, non-limiting examples of detectable labels include a fluorescent dye or biotin.

Further embodiments are a kit for identifying a pathogen in a sample, comprising an array as described herein, including one or more reagents for generating a labeled target, and optionally a hybridization buffer and/or a wash medium.

IV. High Throughput Microarrays for Detecting Target Nucleic Acids

DNA microarray technology has become one of the most important tools for high throughput studies in clinical diagnosis and medical research, with applications in areas including pathogen detection, gene discovery, gene expression, and genetic mapping. Much progress has been made for making high quality microarrays through improving the surface materials and fabrication techniques, but little has been achieved for the qualitative detection of multiple nucleic acids in a single sample. Without such advances, the application of DNA microarray technology is limited in certain areas including clinical diagnosis and medical research. Gene expression studies and clinical diagnosis of pathogens using multiplex PCR reactions and DNA microarray analysis have in the past often been unpredictable and unreliable due to biases produced by differences in amplification conditions and “unbalanced” PCR reactions. Amplification of products other than the desired target nucleic acids can unbalance a PCR reaction by using up one or more primers.

The present disclosure provides a new strategy for a high throughput, microarray-based method of detecting target nucleic acids in a sample, and in particular for multiplex PCR followed by microarray analysis for the qualitative identification of multiple target nucleic acids, including rare targets. In the microarrays described herein, both sense and antisense oligonucleotide probe pairs corresponding to the target molecule are printed on the array. This has the advantage of enabling the detection of balanced versus unbalanced multiplex PCR reactions. In an unbalanced reaction, certain primers participate in side reactions that result in the depletion of dNTPs. This limits the number of primers that can be multiplexed. Prediction and control of side reactions is not generally possible, and they render multiplex results less reliable. As illustrated in the Examples and accompanying figures, in a balanced multiplex PCR reaction, signals from both the sense and antisense probes are the same; in an unbalanced reaction the signals can be different, but at least one target-specific probe still gives a relatively strong signal. By printing sense and antisense probes for each target nucleic acid and examining the microarray for concordance, reliability is improved.

A first application of this method is the rapid, qualitative identification of one or more pathogens in a sample (e.g., an environmental or a biological sample) of interest. For instance, the array-based methods provided herein can be used to rapidly identify the presence of a pathogen in an environmental sample, such as suspect powder. The array-based methods provided herein can also be used to rapidly identify the presence of a pathogen in a biological sample, such as blood.

The disclosed methods can also be used for the detection of rare mRNAs expressed in cells. More broadly, the disclosed methods can be applied to rapidly identify and/or detect any nucleic acid sequence in a sample, and are particularly beneficially used to detect rare nucleic acids.

V. Synthesis of Oligonucleotide Primers and Probes

In vitro methods for the synthesis of oligonucleotides are well known to those of ordinary skill in the art; such methods can be used to produce primers and probes for the disclosed methods. The most common method for in vitro oligonucleotide synthesis is the phosphoramidite method, formulated by Letsinger and further developed by Caruthers (Caruthers et al., Chemical synthesis of deoxyoligonucleotides, in Methods Enzymol. 154:287-313, 1987). This is a non-aqueous, solid phase reaction carried out in a stepwise manner, wherein a single nucleotide (or modified nucleotide) is added to a growing oligonucleotide. The individual nucleotides are added in the form of reactive 3+-phosphoramidite derivatives. See also, Gait (Ed.), Oligonucleotide Synthesis. A practical approach, IRL Press, 1984.

In general, the synthesis reactions proceed as follows: A dimethoxytrityl or equivalent protecting group at the 5′ end of the growing oligonucleotide chain is removed by acid treatment. (The growing chain is anchored by its 3′ end to a solid support such as a silicon bead.) The newly liberated 5′ end of the oligonucleotide chain is coupled to the 3′-phosphoramidite derivative of the next deoxynucleoside to be added to the chain, using the coupling agent tetrazole. The coupling reaction usually proceeds at an efficiency of approximately 99%; any remaining unreacted 5′ ends are capped by acetylation so as to block extension in subsequent couplings. Finally, the phosphite triester group produced by the coupling step is oxidized to the phosphotriester, yielding a chain that has been lengthened by one nucleotide residue. This process is repeated, adding one residue per cycle. See, e.g., U.S. Pat. Nos. 4,415,732, 4,458,066, 4,500,707, 4,973,679, and 5,132,418. Oligonucleotide synthesizers that employ this or similar methods are available commercially (e.g., the PolyPlex oligonucleotide synthesizer from Gene Machines, San Carlos, Calif.). In addition, many companies will perform such synthesis (e.g., Sigma-Genosys, The Woodlands, Tex.; Qiagen Operon, Alameda, Calif.; Integrated DNA Technologies, Coralville, Iowa; and TriLink BioTechnologies, San Diego, Calif.).

Modified nucleotides, such as amino-allyl dNTPs or dNTPs carrying a fluorescent dye (such as Cy3 or Cy5), can be incorporated into an oligonucleotide essentially as described above for non-modified nucleotides. Though most of the examples presented herein refer to the addition of a fluorescent label (particularly Cy3 or Cy5) to the modified nucleotide that is incorporated in an amplified target nucleic acid sequence used in the described methods, other detectable molecules are contemplated.

DNA molecules containing a primary amino group (e.g., attached to the C6 or C2 carbon) can be coupled with a standard peptide or can interact with any intermediate N-hydroxysuccinimide (NHS) ester. In an embodiment disclosed herein, amine modified dT and dC nucleotides are added in place of thymidine and cytidine residues during oligonucleotide synthesis. After deprotection of the modified group, the primary amine on (for instance) the C6 moiety is spatially separated from the oligonucleotide by a spacer arm, and can be reacted with a label molecule or attached to an enzyme or any other reactive peptide or protein. Thus, in particular embodiments, the provided amplified target nucleic acid sequences are linked to a hapten such as biotin, or a fluorescent dye. For instance, any NHS-ester dyes can be used in DNA labeling with the provided amine modified amplified target nucleic acid sequences.

VI. Amplification of Target Nucleic Acids

Nucleic acids are amplified from target gene sequences (e.g., nucleic acids from pathogenic or other microorganisms, or rare nucleic acids expressed in cells) prior to detection. Any nucleic acid amplification method can be used. In one specific, non-limiting example, PCR is used to amplify the target nucleic acid sequences. In other specific, non-limiting examples, RT-PCR, one-step RT-PCR, transcription-mediated amplification (TMA), or ligase chain reaction can be used to amplify the target nucleic acid sequences. Techniques for nucleic acid amplification are well-known to those of skill in the art.

In one embodiment, target DNA sequences are amplified. In another embodiment, target RNA sequences are reverse transcribed prior to amplification using RT-PCR. In one specific, non-limiting example, amplification of a pathogen-specific nucleic acid sequence, for example a specific nucleic acid sequence from Bacillus anthracis, Clostridium botulinum, Yersinia pestis, Variola major, Francisella tularensis, a Filovirus, or an Arenavirus, can be used to detect the presence of one or more of these pathogens in a sample.

Any type of thermal cycler apparatus, for example a PTC- 100® Peltier Thermal Cycler (MJ Research, Inc.; San Francisco, Calif.), a Robocyclerφ 40 Temperature Cycler (Stratagene; La Jolla, Calif.), or a GeneAmp® PCR System 9700 (Applied Biosystems; Foster City, Calif.) can be used to amplify nucleic acid sequences.

In one embodiment, pathogen-specific primers, which specifically bind to unique regions in the genomes of their respective pathogens, are used to produce amplified pathogen-specific nucleic acids. Specific, non-limiting examples of pathogen-specific primers include, but are not limited to, those shown in SEQ ID NOs: 249-428.

At least two primers are utilized in the amplification reaction. One or both of the primers can be end-labeled (for example, radiolabled, fluorescently-labeled, enzymatically-labeled, or biotinylated). In one embodiment, the resulting amplified target nucleic acid sequence is labeled. In another embodiment, the resulting amplified target nucleic acid sequence is labeled by incorporating fluorescent dye-conjugated nucleotides such as Cy3-/Cy5-dUTP/dCTP, or other modified nucleotides, like amino-allyl dUTP, during polymerization of the amplified target nucleic acid sequence (e.g., during PCR or reverse transcription of cDNA from mRNA). Methods for labeling nucleic acids are well known to those of skill in the art. Radioactive and fluorescent labeling methods, as well as other methods known in the art, are suitable for use with the present disclosure.

VII. Arrays for Detection of Amplified Target Nucleic Acid Sequences

Arrays can be used to detect the presence of amplified target nucleic acid sequences, such as target nucleic acid sequences from pathogenic or other microorganisms, or rare nucleic acids expressed in cells, using specific oligonucleotide probes. The arrays described herein are used to detect the presence of amplified target nucleic acids. A pre-determined set of probes are attached to the surface of a solid support for use in detection of the amplified target nucleic acid sequences. The arrays include at least one sense and at least one antisense probe for each target nucleic acid. In one embodiment, each sense probe consists essentially of the complement of the corresponding antisense probe. In another embodiment, each sense probe and its corresponding antisense probe are not complementary. Additionally, if an internal control nucleic acid sequence was amplified in the amplification reaction, an oligonucleotide probe can be included to detect the presence of this amplified nucleic acid.

The oligonucleotide probes attached to the array specifically hybridize to amplified target nucleic acids. In one specific, non-limiting example, the array includes a plurality of pathogen-specific oligonucleotide probes, including at least one sense and at least one antisense probe sequence for each target, printed separately on the array or in a single feature. A hybridization complex is formed when amplified target nucleic acids, for example amplified pathogen-specific target nucleic acids, hybridize to a plurality of cognate oligonucleotide probes chemically linked to a solid support. Specific, non-limiting examples of pathogen-specific sense and antisense oligonucleotide probes are shown in SEQ ID NOs: 429-609. One of skill in the art will be able to identify other pathogen-specific sense and antisense oligonucleotide probes that can be attached to the surface of a solid support for the detection of other amplified pathogen-specific target nucleic acid sequences. For instance, the genomes of numerous pathogens are well known to those of skill in the art and available in public databases. In one embodiment the hybridization complex forms a hybridization pattern.

The arrays of the present disclosure can be prepared by a variety of approaches which are known to those working in the field. Pursuant to one type of approach, the complete oligonucleotide probe sequences are synthesized separately and then attached to a solid support (see U.S. Pat. No. 6,013,789). In another embodiment, the sequences can be synthesized directly onto the support to provide the desired array (see U.S. Pat. No. 5,554,501). Suitable methods for covalently coupling oligonucleotides to a solid support and for directly synthesizing the oligonucleotides onto the support would be readily apparent to those working in the field; a summary of suitable methods can be found in Matson et al., Anal. Biochem. 217:306-10, 1994. In one embodiment, the oligonucleotides are synthesized onto the support using conventional chemical techniques for preparing oligonucleotides on solid supports (for example, see PCT applications WO 85/01051 and WO 89/10977, or U.S. Pat. No. 5,554,501).

The oligonucleotide probes can be attached to the solid support by either the 3′-end of the oligonucleotide or by the 5′-end of the oligonucleotide. In one embodiment, the oligonucleotides are attached to the solid support by the 3′-end. However, one of skill in the art will be able to determine whether the use of the 3′-end or the 5′-end of the oligonucleotide is suitable for attaching to the solid support. In general, the internal complementarity of an oligonucleotide probe in the region of the 3′-end and the 5′-end determines attachment to the support. Generally, the end of the probe with the most internal complementarity is attached to the support, thereby leaving the end with the least internal complementarity to bind to amplified target nucleic acid sequences. The oligonucleotide probe sequences on the array can be directly attached to the solid support. Alternatively, the oligonucleotide probe sequences can be attached to the support by non-probe sequences that serve as spacers or linkers between the probe sequences and the solid support.

In general, suitable characteristics of the material that can be used to form the solid support include, amenability to surface activation, such that upon activation the surface of the support is capable of having a biomolecule (e.g., an oligonucleotide probe) covalently attached thereto, amenability to “in situ” synthesis of biomolecules, and amenability to blocking of areas on the support not occupied by the biomolecules. The solid support can be formed from glass, silicon, metals (e.g., gold), polymer films (e.g., polymers having a substantially non-porous surface); polymer filaments (e.g., mesh and fabrics); polymer beads; polymer foams; polymer frits; and polymer threads. Polymers can include, but are not limited to, cellulosic substrates, such as nitrocellulose, nylon, TEFLON™, polypropylene, polyethylene, polybutylene, polyisobutylene, plybutadiene, polyisoprene, polyvinylpyrrolidine, polytetrafluroethylene, polyvinylidene difluroide, polyfluoroethylene-propylene, polyethylenevinyl alcohol, polymethylpentene, polycholorotrifluoroethylene, polysulfornes, BOPP, hydroxylated BOPP, aminated BOPP, thiolated BOPP, etyleneacrylic acid, thylene methacrylic acid, and blends of copolymers thereof (see U.S. Pat. No. 5,985,567).

In one embodiment, the solid support is glass. In one specific, non-limiting example, the glass is coated with poly-L-lysine. In another embodiment, the solid support is polypropylene. Polypropylene is chemically inert and hydrophobic. Polypropylene has good chemical resistance to a variety of organic acids (for instance, formic acid), organic agents (for instance, acetone or ethanol), bases (for instance, sodium hydroxide), salts (for instance, sodium chloride), oxidizing agents (for instance, peracetic acid), and mineral acids (for instance, hydrochloric acid). Polypropylene also provides a low fluorescence background, which minimizes background interference and increases the sensitivity of the signal of interest. In one specific, non-limiting example, a polypropylene support (e.g., BOPP) is first surface aminated by exposure to an ammonia plasma generated by radiofrequency plasma discharge. The reaction of a phosphoramidite-activated nucleotide with the aminated polypropylene support, followed by oxidation (for example, with iodine), provides a base stable amidate bond to the support.

A suitable array can be produced using automated means to synthesize oligonucleotide probes in the features of the array by laying down the precursors for the four bases in a predetermined pattern. Briefly, a multiple-channel automated chemical delivery system is employed to create oligonucleotide probe populations in parallel rows (corresponding in number to the number of channels in the delivery system) across the solid support. Following completion of oligonucleotide synthesis in a first direction, the support can then be rotated by 90° to permit synthesis to proceed within a second set of rows that are now perpendicular to the first set. This process creates a multiple-channel array whose intersection generates a plurality of discrete cells.

VIII. Assaying Oligonucleotide Arrays

Labeled amplified target nucleic acids, such as nucleic acid sequences from pathogenic or other microorganisms, or rare nucleic acids expressed in cells, are applied to the oligonucleotide array under suitable hybridization conditions to form a hybridization complex. Hybridization conditions for a given combination of oligonucleotide probes and amplified target nucleic acid sequences can be optimized routinely in an empirical manner, so they are close to the Tₘ of the expected duplexes, thereby maximizing the discriminating power of the method. Identification of the location in the array, such as a feature, in which binding occurs, permits a rapid and accurate identification of target nucleic acid sequences present in the amplified material.

The hybridization conditions are selected to permit discrimination between matched and mismatched oligonucleotide probes and amplified target nucleic acid sequences. Hybridization conditions can be chosen to correspond to those known to be suitable in standard procedures for hybridization to filters and then optimized for use with the arrays of the disclosure. For example, conditions suitable for hybridization of one type of target nucleic acid sequence would be adjusted for the use of other target sequences for the array. In particular, temperature is controlled to substantially eliminate formation of duplexes between sequences other than complementary target/probe sequences. A variety of known hybridization solvents can be employed, the choice being dependent on considerations known to one of skill in the art (see, e.g., U.S. Pat. No. 5,981,185).

Once the amplified target nucleic acids have been hybridized with the oligonucleotide probes present on the array, the presence of the hybridization complex is detected. The developing and detection can include the use of a wash medium. Examples of wash media include, sodium saline citrate, sodium saline phosphate, tetramethylammonium chloride, sodium saline citrate in ethylenediaminetetra-acetic, sodium saline citrate in sodium dodecyl sulfate, sodium saline phosphate in ethylenediaminetetra-acetic, sodium saline phosphate in sodium dodecyl sulfate, tetramethylammonium chloride in ethylenediaminetetra-acetic, tetramethylammonium chloride in sodium dodecyl sulfate, or combinations thereof. However, other suitable wash media may also be used. Exemplary wash conditions include, for example, washing with 0.5×SSC, 0.01% SDS for 10 minutes at room-temperature, followed by 0.06×SSC for 10 minutes at room-temperature. The hybridized complex can then be placed on a detection device, such as described below.

IX. Computer Assisted Detection and Analysis of Array Hybridization

The data generated by assaying the disclosed oligonucleotide arrays can be gathered (and analyzed) using known computerized systems. For instance, the array can be read by a computerized “reader” or scanner and quantification of the binding of target sequences to individual addresses on the array carried out using computer algorithms. Likewise, where a control probe has been used, computer algorithms can be used to normalize the hybridization signals in the different features of the array. Such analyses of an array can be referred to as “automated detection,” in that the data is being gathered by an automated reader system.

In the case of labels that emit detectable electromagnetic wave or particles, the emitted light (e.g., fluorescence or luminescence) or radioactivity can be detected by very sensitive cameras, confocal scanners, image analysis devices, radioactive film or a phosphoimager, which capture the signals (such as a color image) from the array. A computer with image analysis software detects this image, and analyzes the intensity of the signal for each probe location in the array. Signals, particularly normalized signals, can be compared between features on a single array (such as between sense and antisense probes), or between arrays (such as a single array that is sequentially interrogated with multiple different target molecule preparations), or between the labels of different targets (or combinations of targets) on a single array.

Computer algorithms also can be used for comparison between features on a single array or on multiple arrays. In addition, the data from an array can be stored in a computer readable form.

Certain examples of automated array readers (scanners) will be controlled by a computer and software programmed to direct the individual components of the reader (e.g., mechanical components such as motors, analysis components such as signal interpretation and background subtraction). Optionally, software also can be provided to control a graphic user interface and one or more systems for sorting, categorizing, storing, analyzing, or otherwise processing the data output of the reader.

To “read” an array, an array that has been assayed with a detectable target to produce binding (e.g., a binding pattern) can be placed into (or onto, or below, etc., depending on the location of the detector system) the reader and a detectable signal indicative of target binding detected by the reader. Those addresses at which the target has bound to an immobilized nucleic acid mixture provide a detectable signal, e.g., in the form of electromagnetic radiation. These detectable signals could be associated with an address identifier signal, identifying the site of the “positive” hybridized spot. The reader gathers information from each of the addresses, associates it with the address identifier signal, and recognizes addresses with a detectable signal as distinct from those not producing such a signal. Certain readers are also capable of detecting intermediate levels of signal, between no signal at all and a high signal, such that quantification of signals at individual addresses is enabled.

Certain readers that can be used to collect data from the arrays, especially those that have been interrogated using a fluorescently tagged molecule, will include a light source for optical radiation emission. The wavelength of the excitation light will usually be in the UV or visible range, but in some situations may be extended into the infra-red range. A beam splitter can direct the reader-emitted excitation beam into the object lens, which for instance may be mounted such that it can move in the x, y and z directions in relation to the surface of the array support. The objective lens focuses the excitation light onto the array, and more particularly onto the (oligonucleotide) targets on the array. Light at longer wavelengths than the excitation light is emitted from addresses on the array that contain fluorescently labeled target molecules (i.e., those addresses containing a nucleic acid molecule within a spot containing a nucleic acid molecule to which the target binds).

In certain embodiments, the array can be movably disposed within the reader as it is being read, such that the array itself moves (for instance, rotates) while the reader detects information from each address. Alternatively, the array may be stationary within the reader while the reader detection system moves across or above or around the array to detect information from the addresses of the array. Specific movable-format array readers are known and described, for instance in U.S. Pat. No. 5,922,617. Examples of methods for generating optical data storage focusing and tracking signals are also known (see, e.g., U.S. Pat. No. 5,461,599).

For the electronics and computer control, a detector (e.g., a photomultiplier tube, avalanche detector, Si diode, or other detector having a high quantum efficiency and low noise) converts the optical radiation into an electronic signal. An op-amp first amplifies the detected signal and then an analog-to-digital converter digitizes the signal into binary numbers, which are then collected by a computer.

X. Oligonucleotide Array Kits

Target-specific oligonucleotide arrays as disclosed herein, such as for detecting nucleic acid sequences from pathogenic or other microorganisms, or rare nucleic acids expressed in cells, can be supplied in the form of a kit for use in nucleic acid analyses. In such a kit, at least one array is provided, wherein the array includes a plurality of target-specific oligonucleotide probes, including both sense and antisense probe sequences. The kit also includes instructions, usually written instructions, to assist the user in probing the array. Such instructions can optionally be provided on a computer readable medium.

Kits may additionally include one or more buffers for use during assay of the provided array. For instance, such buffers may include a low stringency wash, a high stringency wash, and/or a stripping solution. These buffers may be provided in bulk, where each container of buffer is large enough to hold sufficient buffer for several probing or washing or stripping procedures. Alternatively, the buffers can be provided in pre-measured aliquots, which would be tailored to the size and style of array included in the kit. Certain kits may also provide one or more containers in which to carry out array-assaying reactions.

Kits may in addition include either labeled or unlabeled control target molecules, to provide for internal tests of the labeling procedure or interrogation of the oligonucleotide array, or both. The control target molecules may be provided suspended in an aqueous solution or as a freeze-dried or lyophilized powder, for instance. The container(s) in which the controls are supplied can be any conventional container that is capable of holding the supplied form, for instance, microfuge tubes, ampoules, or bottles. In some applications, control probes may be provided in pre-measured single use amounts in individual, typically disposable, tubes, or equivalent containers.

The amount of each control target supplied in the kit can be any particular amount, depending for instance on the market to which the product is directed. For instance, if the kit is adapted for research or clinical use, sufficient control target(s) likely will be provided to perform several controlled analyses of the array. Likewise, where multiple control targets are provided in one kit, the specific targets provided will be tailored to the market and the accompanying kit.

In some embodiments, kits may also include the reagents necessary to carry out one or more amplifications and/or target-labeling reactions. The specific reagents included will be chosen in order to satisfy the end user's needs, depending on the type of target molecule (e.g., DNA or RNA) and the method of labeling (e.g., radiolabel incorporated during target synthesis, attachable fluorescent dye conjugated nucleotides such as Cy3-/Cy5-dUTP/dCTP, or other modified nucleotides, like amino-allyl dUTP).

The subject matter of the present disclosure is further illustrated by the following non-limiting Examples.

### EXAMPLES

### Example 1

### Amplification and Detection of KSHV/EBV-Specific Nucleic Acids in a Sample

This example demonstrates how KSHV/EBV-specific nucleic acids can be amplified from a sample and detected using specific probes on an oligonucleotide array.

Production of Primers and Probes

The KSHV and EBV genomes were screened for virus-specific sequences. These sequences were blasted against the human genome to ensure that no highly similar sequences are present in the human genome. On the basis of this analysis, virus-specific oligonucleotide probes were designed using Primer Quest software (Integrated DNA Technologies, Inc., Coralville, Iowa), that correspond to the target virus-specific genomic sequences (each about 55 base pairs in length, with a Tₘ of 72-73° C. and a percent GC content of 45-50). Both sense and antisense versions of each virus-specific oligonucleotide probe were prepared. Primers with sequences that flank those of the target virus-specific genomic sequences were also prepared (each about 23 base pairs in length, with a Tₘ of 55° C.). All primers and probes were synthesized by Qiagen Operon (Alameda, Calif.) and were dissolved in DEPC treated H₂O at a concentration of 1 μg/μl.

Cell Lines

BC-1, BCLB-1 and B95-8 cells were cultured in RPMI medium plus 15% fetal bovine serum. The cells were infected by either KSHV, EBV or both. Tetradecanoyl phorbol acetate was used to induce viral replication at 12 hour, 24 hour and 36 hour time points.

Nucleic Acid Extraction

Total DNA was extracted from BCLB-1 (infected by KSHV), B95-8 (infected by EBV) and BC-1 (infected by KSHV and EBV) cells using TRIzol reagent (Invitrogen, Carlsbad, Calif.) according to the manufacturer's instructions.

PCR Amplification

PCR amplification was performed in a 25 μl volume containing 2.5 μl of 10× reaction buffer (Invitrogen, Carlsbad, Calif.), 0.125 μl of Taq DNA polymerase (Invitrogen, Carlsbad, Calif.), 400 pg of carrier DNA (ltp 4), 150 μM of each of dATP, dGTP and dCTP, 120 μM of dTTP, 60 μM of amino-allyl dUTP (Sigma, St. Louis, Mo.), 0.20 to 0.50 μM of each forward primer (SEQ ID NOs: 1-31 and 63-93; Table 1 and Table 2), 0.20 to 0.50 μM of each reverse primer (SEQ ID NOs: 32-62 and 94-124; Table 1 and Table 2), sterile double-distilled water, and 1 μl of extracted (template) DNA, containing between 100 ng and 1 pg of DNA.

The PCR thermocycling program consisted of one cycle of 2 minutes at 96° C.; forty cycles of 95° C. for 30 seconds, 55° C. for 30 seconds, and 72° C. for 15 seconds (amplification); and one cycle of 15 seconds at 72° C. (final extension).

TABLE 1

| | | | |
|---|---|---|---|
| Gene | | | SEQ |
| name | | | ID |
| KSHV | Direction | Primer | NO: |

| | | | |
|---|---|---|---|
| ORF 4 | Forward | CATCCATGAAAGCGAAAGG | 1 |
| ORF 6 | Forward | CAGGTTCCCACCTGTTCTG | 2 |
| ORF 7 | Forward | AGTCCCGGGTAAGGCAAG | 3 |
| ORF 8 | Forward | TCAAGGCCATCGAGCTGT | 4 |
| ORF K2 | Forward | TCCCTGAAGCCTCCCTAA | 5 |
| ORF K3 | Forward | AATCACGCCCATGGAACC | 6 |
| ORF 22 | Forward | CCGTCCCTTCCTCCATTC | 7 |
| ORF 31 | Forward | GACACCATCTCGGCCATT | 8 |
| ORF 33 | Forward | ACGCACGTCGAGAATGTG | 9 |
| ORF 34 | Forward | GCATAATTGCGGGTCAGG | 10 |
| ORF 36 | Forward | CCCATGCATTCTGGTGGA | 11 |
| ORF 37 | Forward | GCGTGTCCTCGCAAAAAG | 12 |
| ORF 40 | Forward | CGACGGTGACCTTTGAGG | 13 |
| ORF 43 | Forward | CAAGATGGCGGGCATAGT | 14 |
| ORF 44 | Forward | GGCGTGGACTTTGTGTCC | 15 |
| ORF 49 | Forward | GGGTACGTGGCAGTCTGG | 16 |
| ORF K10 | Forward | GGCCTGTCTGGTTGAGGA | 17 |
| ORF 61 | Forward | CTGTCCCCATGGTCCTTAG | 18 |
| ORF 63 | Forward | GCTGCACTACCCCCAATG | 19 |
| ORF 64 | Forward | TCTTGGTGAGGGGACCAC | 20 |
| ORF K13 | Forward | GCACTTGGCGCTGTAGGT | 21 |
| ORF 72 | Forward | TGACGTCCGTCGCTAAGA | 22 |
| ORF 73 | Forward | TCGTCCTCCTCCTCGTCA | 23 |
| ORF 74 | Forward | TGGAAATGGATTGGTCACCT | 24 |
| ORF 75 | Forward | TGCCCAGACACACCACTG | 25 |
| ORFK1 | Forward | ACTCGGCTTTTGCGACTG | 26 |
| ORFK2 | Forward | CCATCGGCGAGCTTTTTA | 27 |
| ORF26 | Forward | GCAGTGCTACCCCCATTTT | 28 |
| out & in | | | |
| ORFK9-1 & | Forward | TCTGCGCCATTCAAAACA | 29 |
| K9-3 | | | |
| ORF72 | Forward | TCCAGAATGCGCAGATCA | 30 |
| ORF74 | Forward | CCACAGGCTTGTGCAGACT | 31 |
| ORF 4 | Reverse | TGTACGTGTCGGTGCGTTA | 32 |
| ORF 6 | Reverse | TTGGAGCATCTTCCACAGG | 33 |
| ORF 7 | Reverse | TCTGTCTCCGTCGCAGGT | 34 |
| ORF 8 | Reverse | CAGATCCTCGCGCAAACC | 35 |
| ORF K2 | Reverse | TGGAGCTTCTGACGAAGACC | 36 |
| ORF K3 | Reverse | GGGCGATGGGGCTTATAG | 37 |
| ORF 22 | Reverse | GCAGCTGTCGGTGAGGAC | 38 |
| ORF 31 | Reverse | GCTTGGCAATGCAGTCGT | 39 |
| ORF 33 | Reverse | GCCCAGGGCCTTAAGTTT | 40 |
| ORF 34 | Reverse | TGGTTGAGTCCATTCTCCTTG | 41 |
| ORF 36 | Reverse | GCAGCAGGTTGCACTTCA | 42 |
| ORF 37 | Reverse | TGAAGCGGGCTTTAATACTGA | 43 |
| ORF 40 | Reverse | CGTTCGACTGGCAGGAGT | 44 |
| ORF 43 | Reverse | CCAGCGTGACTCAGGAGAT | 45 |
| ORF 44 | Reverse | GACGGCCGAATCTCACTG | 46 |
| ORF 49 | Reverse | GGCCCCTTAAAGATCACCTG | 47 |
| ORF K10 | Reverse | TGGACCAGAACAATCTTTAGTGC | 48 |
| ORF 61 | Reverse | GCCTACAGACGGTCCAAAG | 49 |
| ORF 63 | Reverse | TCCGAGGGTATGCCAGAG | 50 |
| ORF 64 | Reverse | TTTTGGATGCCCACAAGG | 51 |
| ORF K13 | Reverse | GAGCTGTGTGCGAGGGATA | 52 |
| ORF 72 | Reverse | GCGGCAGACTCCTTTTCC | 53 |
| ORF 73 | Reverse | GCGAGGATAATGGGGACA | 54 |
| ORF 74 | Reverse | GGGAAACAAAAACATCAACACT | 55 |
| ORF 75 | Reverse | ACCATCGCCACCACTCCT | 56 |
| ORFK1 | Reverse | TGGCTGTGCACACAAGGT | 57 |
| ORFK2 | Reverse | GCCCGCTGCTATTTTTCA | 58 |
| ORF26 | Reverse | AATAGCGTGCCCCAGTTG | 58 |
| out & in | | | |
| ORFK9-1 & | Reverse | GGATTGGATAGTATGTCAAGTCAACA | 60 |
| K9-3 | | | |
| ORF72 | Reverse | TCCGCAGGATACCCACTC | 61 |
| ORF74 | Reverse | ACAGTGCAGCGGATGTCA | 62 |

TABLE 2

| | | | |
|---|---|---|---|
| Gene | | | SEQ |
| name | | | ID |
| EBV | Direction | Primer | NO: |

| | | | |
|---|---|---|---|
| BNRF1 | Forward | GGGGCCCGTTTATTATGG | 63 |
| BYRF1 | Forward | GCGTTACATGGGGGACAA | 64 |
| BFRF3 | Forward | TTCGGGAGGCTCAAAGAA | 65 |
| BPLF1 | Forward | ACCGGAAGGGCTCAGAGT | 66 |
| BORF2 | Forward | AGACCCCGAGGCTGATGT | 67 |
| BMRF1 | Forward | AGCCGTCCTGTCCAAGTG | 68 |
| BSLF1 | Forward | TGACTGGCCTCAGCCCTA | 69 |
| BLRF1 | Forward | CCTGACTGAAGCCCAGGA | 70 |
| BRLF1 | Forward | TGGTGGCAGGAATCATCA | 71 |
| BRRF1 | Forward | CTGTGGCCCTCTGCAAGT | 72 |
| BKRF1 | Forward | TGGAAAGCATCGTGGTCA | 73 |
| BKRF4 | Forward | TGTCGGACGAGGAGGAAG | 74 |
| BBRF1 | Forward | CTTTGGGAAAGCGAGCTG | 75 |
| BBLF1 | Forward | TATTCGAGCCCCTCGTTG | 76 |
| BGLF5 | Forward | GCTGTCTGCCACCAGGTC | 77 |
| BGLF4 | Forward | TGCAGGCCGACAGGTAGT | 78 |
| DNA pkg. | Forward | CTTCGTGCACACCAAGGA | 79 |
| BDLF3 | Forward | CCCAGCCGCAAATATCAG | 80 |
| BDLF2 | Forward | GCGAGTAGGGCCAGGAAC | 81 |
| BDLF1 | Forward | CGGGGGCATAACACTGAG | 82 |
| BXLF2 | Forward | GCCAAAGACCAGGCTCAA | 83 |
| BXLF1 | Forward | CCCTTGCCACCATTCTTTT | 84 |
| BILF2 | Forward | ATGCGCAAGGGTCACATT | 85 |
| BALF4 | Forward | GCGTCAGCCCATCTTTTG | 86 |
| BALF2 | Forward | GCCACAGGTACAGGCTTGA | 87 |
| EBV W | Forward | AGCGGGTGCAGTAACAGG | 88 |
| BLRF2 | Forward | AGCCGCTTACAGCTCGAC | 89 |
| EBNA-1 | Forward | TGGAAAGCATCGTGGTCA | 90 |
| EBER-2 | Forward | GGACCTACGCTGCCCTAGA | 91 |
| EBNA-2 | Forward | CTACCAGAGGGGGCCAAG | 92 |
| LMP-1 | Forward | GGTGCGCCTAGGTTTTGA | 93 |
| BNRF1 | Reverse | CGCCACTAGCAGCAGGTT | 94 |
| BYRF1 | Reverse | CCGTGTTTTCCCCAACAA | 95 |
| BFRF3 | Reverse | CTTGTCTATGGCGCGTTG | 96 |
| BPLF1 | Reverse | TGACACCATCCCCGTCTC | 97 |
| BORF2 | Reverse | CCGGTGCATCTGGAAGAA | 98 |
| BMRF1 | Reverse | CACAGCGTCAGGGGAGAC | 99 |
| BSLF1 | Reverse | GCCTGAGGCATACCCACA | 100 |
| BLRF1 | Reverse | AATCCGTCAGCAGCGTGT | 101 |
| BRLF1 | Reverse | TGCAATTTTTGGGCCATT | 102 |
| BRRF1 | Reverse | CATGCCATCCTGGGATATT | 103 |
| BKRF1 | Reverse | GGCGACCCAAGTTCCTTC | 104 |
| BKRF4 | Reverse | CCCTCAGATGGGTCTTCG | 105 |
| BBRF1 | Reverse | CCACCGGTGAAAGCTCTG | 106 |
| BBLF1 | Reverse | TGGACGGGGGAATAATCA | 107 |
| BGLF5 | Reverse | GCTCGTCCTACCGTGGAG | 108 |
| BGLF4 | Reverse | GCAGATAATGCCACGGTCA | 109 |
| DNA pkg. | Reverse | TGGTGTTGGAGACGGTGA | 110 |
| BDLF3 | Reverse | CAAAGGGACGTCCAATGC | 111 |
| BDLF2 | Reverse | AGCGAGGTATGCGGTGAG | 112 |
| BDLF1 | Reverse | AGACGGTCCCGGACTACG | 113 |
| BXLF2 | Reverse | GTGGCCCTGTCCATCAAC | 114 |
| BXLF1 | Reverse | CCGGAGCTTCATGTACCAG | 115 |
| BILF2 | Reverse | CTTCCAACAACGGAACTCA | 116 |
| BALF4 | Reverse | CGGACTCCGTGACCAACC | 117 |
| BALF2 | Reverse | CTGTGCCCCAGGAACATC | 118 |
| EBV W | Reverse | GCCCATTCGCCTCTAAAGT | 119 |
| BLRF2 | Reverse | TTCCATTTCATTGCGGGTA | 120 |
| EBNA-1 | Reverse | GGCGACCCAAGTTCCTTC | 121 |
| EBER-2 | Reverse | CAGACACCGTCCTCACCA | 122 |
| EBNA-2 | Reverse | GCCCTGGAGAGGTCAGGT | 123 |
| LMP-1 | Reverse | ACACCACCACGATGACTCC | 124 |

Purification of PCR Products and Dye Conjugation

Following the PCR, amplified virus-specific genomic sequences were purified with the QiaQuick PCR Purification Kit (Qiagen, Valencia, Calif.) according to the manufacturer's instructions, vacuum-dried and eluted in 9 μl of sterile double-distilled water. To the eluate, 1 μl of 1M sodium bicarbonate buffer pH 9.0 was added, followed by 4.5 μl NHS-cye dye (Cy3 or Cy5, Pharmacia, Piscataway, N.J.). The conjugation mixture was then incubated at room-temperature for one hour in the dark and quenched with 4 M hydroxylamine.

To remove unincorporated cye dyes, the conjugation mixture was purified with the QiaQuick PCR Purification Kit (Qiagen, Valencia, Calif.) according to the manufacturer's instructions, and Cy3- and Cy5-labeled products were combined. To the combined Cy3- and Cy5-labeled products, 60 μl of sterile double-distilled water was added, followed by 500 μl of PB buffer (Qiagen, Valencia, Calif.). The mixture was applied to a QiaQuick column and spun at 13,000 rpm for 1 minute. The flow-through was reloaded onto the same column for a second spin and then discarded. The column was washed twice with 500 μl of PE buffer (Qiagen, Valencia, Calif.) and spun at 13,000 rpm for 1 minute. Dye-conjugated amplified virus-specific genomic sequences were eluted from the column with 20 μl of EB buffer (Qiagen, Valencia, Calif.) for 1 minute room-temperature, followed by centrifugation at 13,000 rpm for 1 minute. The elution step was repeated two additional times.

Production of Oligonucleotide Microarrays

Oligonucleotide probes (SEQ ID NOs: 125-248; Table 3 and Table 4) were solubilized in 50% DMSO and spotted in duplicate on poly-L-lysine coated slides or Ultra GAPS slides (Coming, Acton, Mass.) using an OmniGrid arrayer (Gene Machines, San Carlos, Calif.) at a concentration of 50 μM. Slides were processed for hybridization according to Xiang and Brownstein (Fabrication of cDNA microarrays, in: Methods in Molecular Biology, vol. 224, Functional Genomics, Methods and Protocols, M.J. Brownstein and A. Khodursky (eds.), Humana Press Inc., 2003).

TABLE 3

| Gene | | | SEQ |
|---|---|---|---|
| name | | | ID |
| KSHV | Direction | Oligonucleotide | NO: |
| ORF 4 | Forward | CGTCTACACCCACTTCCCAAGATGATGCTACGCCTTCAATACCTAGTGTACAGAC | 125 |
| ORF 6 | Forward | GTACAATGACCTAGAGATTCTCGGAAACTTTGCCACCTTCAGGGAGAGAGAGGAG | 126 |
| ORF 7 | Forward | GAGAGGTGACCAGATCTGTCCTGGAAATCTCAAACCTGATCTATTGGAGCTCTGG | 127 |
| ORF 8 | Forward | GTAGCGTGTTTGACCTGGAGACGATGTTCAGGGAGTACAACTACTACACACATCG | 128 |
| ORF K2 | Forward | GTGGACTGTAGTGCGTCTTAGTCAGCTTATTGAGCTCTTCCTGTATGTCCCATCC | 129 |
| ORF K3 | Forward | CTGTGGAAGGATATCAACTAGAGAGGAGGGTCCAGCCTTATTATGGCAGGAGAC | 130 |
| ORF 22 | Forward | GCTATGGTCGTCGAACATATGTATACCGCCTACACTTATGTGTACACACTCGGCG | 131 |
| ORF 31 | Forward | GGTACGCCATGGTCTGTAGCATGTATCTGCACGTTATCGTCTCCATCTATTCGAC | 132 |
| ORF 33 | Forward | CCTGGGTCCTCTTACGAATGTCTGACTACTTCAGCCGCTTGCTGATATATGAGTG | 133 |
| ORF 34 | Forward | CAACTACGGGCGACTATCTAATCATCCCATCGTATGACATACCGGCGATCATCAC | 134 |
| ORF 36 | Forward | GACTGCTAGGATTCGTGCAGCCTTGCATACCCTGTAGATCGATTGTGTATCCTAG | 135 |
| ORF 37 | Forward | CACAACTCGACCACGGAGTCTGACGTCTACGTACTTACTGATCCTCAAGATACTC | 136 |
| ORF 40 | Forward | GAACGTGGCACAGCTCTATCTATAGGGAATGTGCGATCTCGGCTATCGAGATATG | 137 |
| ORF 43 | Forward | CAGTCATAGTCTATGCTCACCTCTGAGTAGCCCGGAATATAGAGGGCGCTTAAAC | 138 |
| ORF 44 | Forward | GCTCCACGGTCTAGTGGCATACGCATCCACTATAGACACCTATATAATCCAGGG | 139 |
| ORF 49 | Forward | GACGGACAGGGTATCTAACTCCTGAAGTATCTGATCCCAGGACGGGTAATGATAC | 140 |
| ORF K10 | Forward | CTTCTTCCCACGTACATATATCCTCTCCTTGAAGGTTCGAGAGCGTAAGAGGGAG | 141 |
| ORF 61 | Forward | GTATCATACAACCTCACGGCCGATAGGTAGCCACAGTTAAGTGTGTCCTCGTAAG | 142 |
| ORF 63 | Forward | CACTTCTCCGTTACAGGACTGGCTTATAGTCGCCTATGGTAACAAGGAAGGACTG | 143 |
| ORF 64 | Forward | CACTCCTAGGATACGGGTCGGTGCAGGACTACAAGGAGACGGTACAGATAATATC | 144 |
| ORF K13 | Forward | CATACAGTACACCCAGTGTAAGAATGTCTGTGGTGTGCTGCGAGACCCTATAGTG | 145 |
| ORF 72 | Forward | CCTCTGTTCGCCACGCCAACTTCTCAAGGAGTTCTTTCTCCTGGTCTATAAGTTC | 146 |
| ORF 73 | Forward | CGTCCTCCTCATCTGTCTCCTGCTCCTCCTCATCATCCTTATTGTCATTGTCATC | 147 |
| ORF 74 | Forward | GGAGCGATAGATATACTGCTCCTGGGTATCTGCCTAAACTCGCTGTGTCTTAGC | 148 |
| ORF 75 | Forward | GGGATCATCCTTCTCAGGGAGATGCATTCTTTGGAAGTAGTGGTAGAGATGGAGC | 149 |
| ORFK1 | Forward | CAATCTGGGCATCGACAGAGCATTTGGATTACATGGCGTGCACAACCTGTCTTAC | 150 |
| ORFK2 | Forward | GGCAGCTAGTCTCATTAAATCCTATTAACCCGCAGTGATCAGTATCGTTGATGGC | 151 |
| ORF26 | Forward | AGCCGAAAGGATTCCACCATTGTGCTCGAATCCAACGGATTTGACCTCGTGTTCC | 152 |
| out & in | | | |
| ORFK9-1 & | Forward | CTGGTATACGGAAGCGGGTGCGCTCTTCGTCTTCCCACTCTACTCCGGGAAATTT | 153 |
| K9-3 | | | |
| ORF72 | Forward | CTGTAGAACGGAAACATCGCATCCCAATATGCTTGCCAGCTGAGGAACTACC | 154 |
| ORF74 | Forward | TTCAGTGTTGTGTGCGTCAGTCTAGTGAGGTACCTCCTGGTGGCATATTCTACG | 155 |
| ORF 4 | Reverse | GTCTGTACACTAGGTATTGAAGGCGTAGCATCATCTTGGGAAGTGGGTGTAGACG | 156 |
| ORF 6 | Reverse | CTCCTCTCTCTCCCTGAAGGTGGCAAAGTTTCCGAGAATCTCTAGGTCATTGTAC | 157 |
| ORF 7 | Reverse | CCAGAGCTCCAATAGATCAGGTTTGAGATTTCCAGGACAGATCTGGTCACCTCTC | 158 |
| ORF 8 | Reverse | CGATGTGTGTAGTAGTTGTACTCCCTGAACATCGTCTCCAGGTCAAACACGCTAC | 159 |
| ORF K2 | Reverse | GGATGGGACATACAGGAAGAGCTCAATAAGCTGACTAAGACGCACTACAGTCCAC | 160 |
| ORF K3 | Reverse | GTCTCCTGCCATAATAAGGCTGGACCCTCCTCTCTAGTTGATATCCTTCCACAG | 161 |
| ORF 22 | Reverse | CGCCGAGTGTGTACACATAAGTGTAGGCGGTATACATATGTTCGACGACCATAGC | 162 |
| ORF 31 | Reverse | GTCGAATAGATGGAGACGATAACGTGCAGATACATGCTACAGACCATGGCGTACC | 163 |
| ORF 33 | Reverse | CACTCATATATCAGCAAGCGGCTGAAGTAGTCAGACATTCGTAAGAGGACCCAGG | 162 |
| ORF 34 | Reverse | GTGATGATCGCCGGTATGTCATACGATGGGATGATTAGATAGTCGCCCGTAGTTG | 165 |
| ORF 36 | Reverse | CTAGGATACACAATCGATCTACAGGGTATGCAAGGCTGCACGAATCCTAGCAGTC | 166 |
| ORF 37 | Reverse | GAGTATCTTGAGGATCAGTAAGTACGTAGACGTCAGACTCCGTGGTCGAGTTGTG | 167 |
| ORF 40 | Reverse | CATATCTCGATAGCCGAGATCGCACATTCCCTATAGATAGAGCTGTGCCACGTTC | 168 |
| ORF 43 | Reverse | GTTTAAGCGCCCTCTATATTCCGGGCTACTCAGAGGTGAGCATAGACTATGACTG | 169 |
| ORF 44 | Reverse | CCCTGGATTATATAGGTGTCTATAGTGGATGCGTATGCCACTAGACCGTGGAGC | 170 |
| ORF 49 | Reverse | GTATCATTACCCGTCCTGGGATCAGATACTTCAGGAGTTAGATACCCTGTCCGTC | 171 |
| ORF K10 | Reverse | CTCCCTCTTACGCTCTCGAACCTTCAAGGAGAGGATATATGTACGTGGGAAGAAG | 172 |
| ORF 61 | Reverse | CTTACGAGGACACACTTAACTGTGGCTACCTATCGGCCGTGAGGTTGTATGATAC | 173 |
| ORF 63 | Reverse | CAGTCCTTCCTTGTTACCATAGGCGACTATAAGCCAGTCCTGTAACGGAGAAGTG | 174 |
| ORF 64 | Reverse | GATATTATCTGTACCGTCTCCTTGTAGTCCTGCACCGACCCGTATCCTAGGAGTG | 175 |
| ORF K13 | Reverse | CACTATAGGGTCTCGCAGCACACCACAGACATTCTTACACTGGGTGTACTGTATG | 176 |
| ORF 72 | Reverse | GAACTTATAGACCAGGAGAAAGAACTCCTTGAGAAGTTGGCGTGGCGAACAGAGG | 177 |
| ORF 73 | Reverse | GATGACAATGACAATAAGGATGATGAGGAGGAGCAGGAGACAGATGAGGAGGACG | 178 |
| ORF 74 | Reverse | GCTAAGACACAGCGAGTTTAGGCAGATACCCAGGAGCAGTATATCTATCGCTCC | 179 |
| ORF 75 | Reverse | GCTCCATCTCTACCACTACTTCCAAAGAATGCATCTCCCTGAGAAGGATGATCCC | 180 |
| ORFK1 | Reverse | GTAAGACAGGTTGTGCACGCCATGTAATCCAAATGCTCTGTCGATGCCCAGATTG | 181 |
| ORFK2 | Reverse | GCCATCAACGATACTGATCACTGCGGGTTAATAGGATTTAATGAGACTAGCTGCC | 182 |
| ORF26 | Reverse | GGAACACGAGGTCAAATCCGTTGGATTCGAGCACAATGGTGGAATCCTTTCGGCT | 183 |
| out & in | | | |
| ORFK9-1 & | Reverse | AAATTTCCCGGAGTAGAGTGGGAAGACGAAGAGCGCACCCGCTTCCGTATACCAG | 184 |
| K9-3 | | | |
| ORF72 | Reverse | GGTAGTTCCTCAGCTGGCAAGCATATTGGGATGCGATGTTTCCGTTCTACAG | 185 |
| ORF74 | Reverse | CGTAGAATATGCCACCAGGAGGTACCTCACTAGACTGACGCACACAACACTGAA | 186 |

TABLE 4

| Gene | | | SEQ |
|---|---|---|---|
| name | | | ID |
| EBV | Direction | Oligonucleotide | NO: |
| BNRF1 | Forward | GATGGAGAGGCAAACATACAGGAGGAAAGGCTATATGAGCTACTCTCTGACCCAC | 187 |
| BYRF1 | Forward | GATAGTCTTGGAAACCCGTCACTCTCAGTAATTCCCTCGAATCCCTACCAGGAAC | 188 |
| BFRF3 | Forward | GTTACCTGGTATTTCTGACATCCCAGTTCTGCTACGAAGAGTACGTGCAGAGGAC | 189 |
| BPLF1 | Forward | CCCTGACCTGTCCCAGGGTCTTCAGGTTAAACAGATATTGAGAGGAGACAAAGAG | 190 |
| BORF2 | Forward | CTTAAGGCCGAGTCAGTTACACACACAGTAGCCGAATATCTGGAGGTCTTCTCTG | 191 |
| BMRF1 | Forward | CTCATCTCAAGGGAGGAGTGCTGCAGGTAAACCTTCTGTCTGTAAACTATGGAGG | 192 |
| BSLF1 | Forward | CTGACTCATGAAGGTGACCGTGATGGCCTGTGATGTGTAGTAGAGTACCAGAAAC | 193 |
| BLRF1 | Forward | CTCCATCTGGGCACTTCTGACGCTTGTCTTAGTCATTATAGCCTCAGCCATCTAC | 194 |
| BRLF1 | Forward | GAATCTGTCAGTGACCACTATCAGGTGGTCTAACACGTAGCGCATCACTATAGGG | 195 |
| BRRF1 | Forward | CTATAACTACATTCAGGGATCTATAGCCACCATCTCCCAGCTTCTGCACCTCGAG | 196 |
| BKRF1 | Forward | CATTGCAGAAGGTTTAAGAGCTCTCCTGGCTAGGAGTCACGTAGAAAGGACTACC | 197 |
| BKRF4 | Forward | GAGGACGTGAGTGACACTGATGAGTCTGACTACTCAGATGAAGACGAGGAGATTG | 198 |
| BBRF1 | Forward | GCAGCGTCTCTACGTCAGATACTCGTCAGACACGATCTCTATATTATTGGGCCC | 199 |
| BBLF1 | Forward | CCTCCCTTCTTCGGCCGCTATTAGCTTAGTAGTCTCCAGGTTAAACTCCTCATAG | 200 |
| BGLF5 | Forward | CTTACGGACATCTTTAAGATTCCAGGCCTCATCCTGCGTCAACAGATAGTCACCC | 201 |
| BGLF4 | Forward | GAATCATGTCACACACCATGAGCTCGTGATACAGCTCCGTCACAGAGTCATAGAG | 202 |
| DNA pkg. | Forward | CCATGTACCTCCTGACTAATGAGAAGTCCAAGGCCTTTGAGAGGCTCATCTACG | 203 |
| BDLF3 | Forward | CAAACACCAGTGTCCAGAGAGGAAGACCGTAAGATAAAGATGGCTGCCTCTCATC | 204 |
| BDLF2 | Forward | GGCTCAGCTAGGGTCTCTGCCTCTCCATCATAGACATCTTCCTTGAATCTCATTC | 205 |
| BDLF1 | Forward | CGTAGGTCTGACCTGGAACAATCTTGGTGAGTATCAAACTGTCCACGCTAACCTC | 206 |
| BXLF2 | Forward | CTCATCTCCCTTCTCGGTCACTCGCTTGTAGGTGCCCATCAGAAATTTAGAAGTC | 207 |
| BXLF1 | Forward | GAGAAGAGGGCCTGCGGAAATTAGACTCATCCTCAGACTCACAGTCAGATTTGTC | 208 |
| BILF2 | Forward | GGGATTATCAGAGAGACGGAGGTGTTGGAGTCATTTACCCATTCTAGGGTAAGGC | 209 |
| BALF4 | Forward | GTAGATGGTATCCATCTGGTCAGTTTCGTAGCTGTCAACGGAGAACTTCTCCTCG | 210 |
| BALF2 | Forward | CGTTGATGATGTAGTTCTCCCTCCTGGTAGTGGACTTGATGAAGCTGTTCTGGAG | 211 |
| EBV W | Forward | GATTTGGACCCGAAATCTGACACTTTAGAGCTCTGGAGGACTTTAAA | 212 |
| BLRF2 | Forward | GGCCGTTTGGCGTCTCAGGCTATGAAGAAGATTGAAGACAAGGTTCGGAAATCTG | 213 |
| EBNA-1 | Forward | CATTGCAGTAGGTTTAAGAGCTCTCCTGGCTAGGAGTCACGTAGAAAGGACTACC | 214 |
| EBER-2 | Forward | TTTGCTAGGGAGGAGACGTGTGTGGCTGTAGCCACCCGTCCCGGGTACAAGT | 215 |
| EBNA-2 | Forward | GTAGAAGGGTCCTCGTCCAGCAAGAAGAGGAGGTGGTTAGCGGTTCACCTTCAG | 216 |
| LMP-1 | Forward | CTCGTTGGAGTTAGAGTCAGATTCATGGCCAGAATCATCGGTAGCTTGTTGAGGG | 217 |
| BNRF1 | Reverse | GTGGGTCAGAGAGTAGCTCATATAGCCTTTCCTCCTGTATGTTTGCCTCTCCATC | 218 |
| BYRF1 | Reverse | GTTCCTGGTAGGGATTCGAGGGAATTACTGAGAGTGACGGGTTTCCAAGACTATC | 219 |
| BFRF3 | Reverse | GTCCTCTGCACGTACTCTTCGTAGCAGAACTGGGATGTCAGAAATACCAGGTAAC | 220 |
| BPLF1 | Reverse | CTCTTTGTCTCCTCTCAATATCTGTTTAACCTGAAGACCCTGGGACAGGTCAGGG | 221 |
| BORF2 | Reverse | CAGAGAAGACCTCCAGATATTCGGCTACTGTGTGTGTAACTGACTCGGCCTTAAG | 222 |
| BMRF1 | Reverse | CCTCCATAGTTTACAGACAGAAGGTTTACCTGCAGCACTCCTCCCTTGAGATGAG | 223 |
| BSLF1 | Reverse | GTTTCTGGTACTCTACTACACATCACAGGCCATCACGGTCACCTTCATGAGTCAG | 224 |
| BLRF1 | Reverse | GTAGATGGCTGAGGCTATAATGACTAAGACAAGCGTCAGAAGTGCCCAGATGGAG | 225 |
| BRLF1 | Reverse | CCCTATAGTGATGCGCTACGTGTTAGACCACCTGATAGTGGTCACTGACAGATTC | 226 |
| BRRF1 | Reverse | CTCGAGGTGCAGAAGCTGGGAGATGGTGGCTATAGATCCCTGAATGTAGTTATAG | 227 |
| BKRF1 | Reverse | GGTAGTCCTTTCTACGTGACTCCTAGCCAGGAGAGCTCTTAAACCTTCTGCAATG | 228 |
| BKRF4 | Reverse | CAATCTCCTCGTCTTCATCTGAGTAGTCAGACTCATCAGTGTCACTCACGTCCTC | 229 |
| BBRF1 | Reverse | GGGCCCAATAATATAGAGATCGTGTCTGACGAGTATCTGACGTAGAGACGCTGC | 230 |
| BBLF1 | Reverse | CTATGAGGAGTTTAACCTGGAGACTACTAAGCTAATAGCGGCCGAAGAAGGGAGG | 231 |
| BGLF5 | Reverse | GGGTGACTATCTGTTGACGCAGGATGAGGCCTGGAATCTTAAAGATGTCCGTAAG | 232 |
| BGLF4 | Reverse | CTCTATGACTCTGTGACGGAGCTGTATCACGAGCTCATGGTGTGTGACATGATTC | 233 |
| DNA pkg. | Reverse | CGTAGATGAGCCTCTCAAAGGCCTTGGACTTCTCATTAGTCAGGAGGTACATGG | 234 |
| BDLF3 | Reverse | GATGAGAGGCAGCCATCTTTATCTTACGGTCTTCCTCTCTGGACACTGGTGTTTG | 235 |
| BDLF2 | Reverse | GAATGAGATTCAAGGAAGATGTCTATGATGGAGAGGCAGAGACCCTAGCTGAGCC | 236 |
| BDLF1 | Reverse | GAGGTTAGCGTGGACAGTTTGATACTCACCAAGATTGTTCCAGGTCAGACCTACG | 237 |
| BXLF2 | Reverse | GACTTCTAAATTTCTGATGGGCACCTACAAGCGAGTGACCGAGAAGGGAGATGAG | 238 |
| BXLF1 | Reverse | GACAAATCTGACTGTGAGTCTGAGGATGAGTCTAATTTCCGCAGGCCCTCTTCTC | 239 |
| BILF2 | Reverse | GCCTTACCCTAGAATGGGTAAATGACTCCAACACCTCCGTCTCTCTGATAATCCC | 240 |
| BALF4 | Reverse | CGAGGAGAAGTTCTCCGTTGACAGCTACGAAACTGACCAGATGGATACCATCTAC | 241 |
| BALF2 | Reverse | CTCCAGAACAGCTTCATCAAGTCCACTACCAGGAGGGAGAACTACATCATCAACG | 242 |
| EBV W | Reverse | TTTAAAGTCCTCCAGAGCTCTAAAGTGTCAGATTTCGGGTCCAAATC | 243 |
| BLRF2 | Reverse | CAGATTTCCGAACCTTGTCTTCAATCTTCTTCATAGCCTGAGACGCCAAACGGCC | 244 |
| EBNA-1 | Reverse | GGTAGTCCTTTCTACGTGACTCCTAGCCAGGAGAGCTCTTAAACCTTCTGCAATG | 245 |
| EBER-2 | Reverse | ACTTGTACCCGGGACGGGTGGCTACAGCCACACACGTCTCCTCCCTAGCAAA | 246 |
| EBNA-2 | Reverse | CTGAAGGTGAACCGCTTACCACCTCCTCTTCTTGCTGGACGAGGACCCTTCTAC | 247 |
| LMP-1 | Reverse | CCCTCAACAAGCTACCGATGATTCTGGCCATGAATCTGACTCTAACTCCAACGAG | 248 |

Hybridization of Dye-Conjugated PCR Products With the Microarray

Eluted dye-conjugated amplified virus-specific genomic sequences were vacuum-dried down and eluted in 9.5 μl of sterile double-distilled water. To the eluate, 2 μl of poly A, 1 μl of yeast tRNA, 2.25 μl of 20% SCC, and 0.25 μl of 10% SDS was added. The mixture was heated at 98° C. for 2 minutes, snap cooled on wet ice and centrifuged for 5 minutes. The oligonucleotide microarrays were prehybridized with prehybridization buffer (5×SSC, 0.1% SDS and 1% BSA) at 42° C. for 45 minutes and then dried at 800 rpm for 2 minutes. The 4 μl denatured samples were then spotted onto the oligonucleotide microarrays. The slides were covered with a cover slip and incubated for 30 minutes at 65° C. on a water bath.

The slides were washed sequentially with 1×SSC, 0.1% SDS for 10 minutes at room-temperature and 0.1×SSC for 10 minutes at room-temperature, then dried by centrifuging at 800 rpm for 2 minutes.

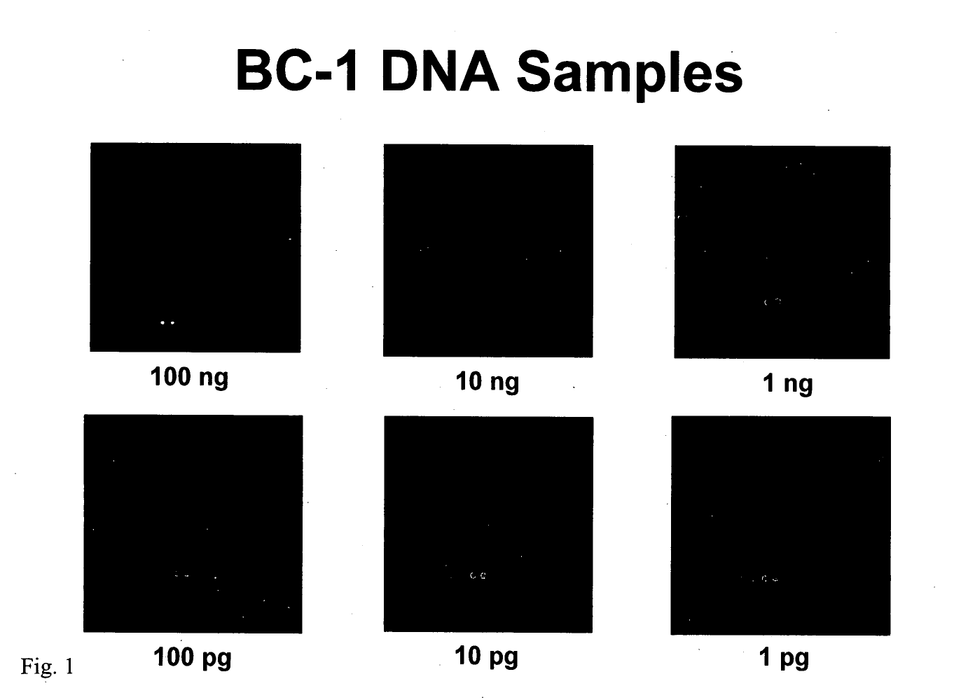

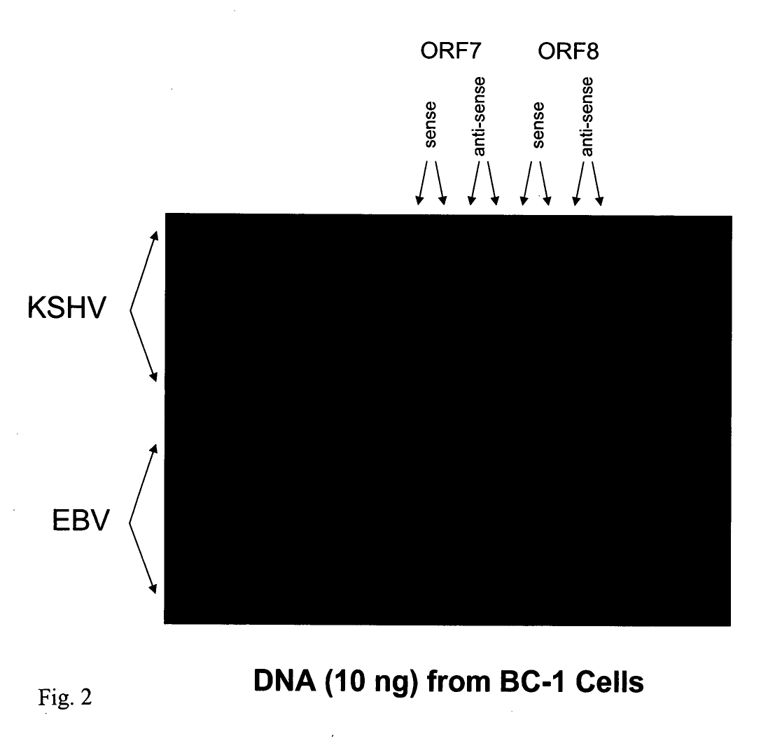

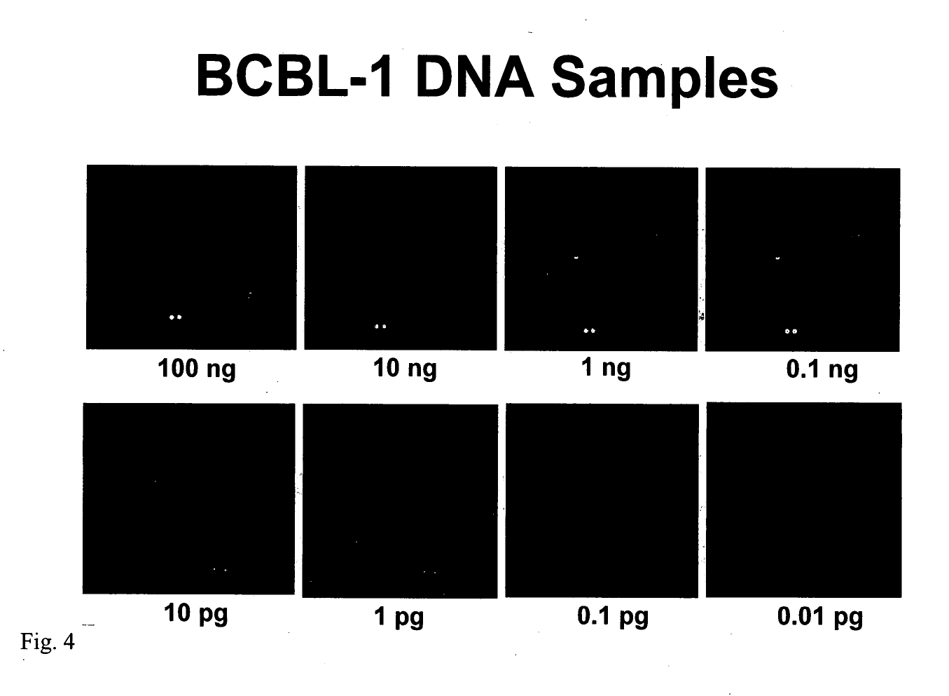

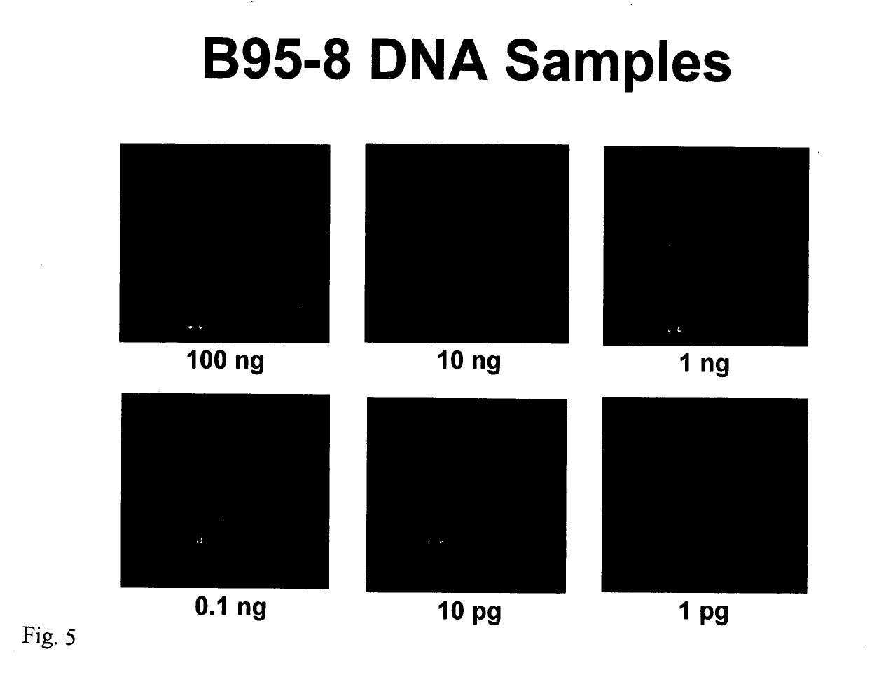

The slides were scanned on a GenePix 4000A scanner (Axon, Foster City, Calif.) at 10 μm resolution to capture tif images (FIGS. 1, 2, 4, and 5). The PMT voltage was 680 for detection at 635 nm and 700 for detection at 535 nm.

### Example 2

### Amplification and Detection of Pathogen-Specific Nucleic Acids in a Sample

This example demonstrates how pathogen-specific nucleic acids can be amplified from a sample and detected using specific probes on an oligonucleotide array.

Production of Primers and Probes

The genomes of the following pathogens were screened for pathogen-specific sequences: Variola major, Vaccinia virus, Ebola virus, Marburg virus, Bacillus anthracis, Clostridium botulinum, Francisella tularensis, Lassa Fever virus, Lymphocytic Choriomeningitis virus, Junin virus, Machupo virus, Guanarito virus, Crimean-Congo Hemorrhagic Fever virus, Hantavirus, Rift Valley Fever virus, Dengue virus, Yersinia pestis, West Nile virus, and SARS-CoV. These sequences were blasted against the human genome to ensure that no highly similar sequences are present in the human genome. On the basis of this analysis, pathogen-specific oligonucleotide probes were designed using Primer Quest software (Integrated DNA Technologies, Inc., Coralville, Iowa), that correspond to the target pathogen-specific genomic sequences (each about 55 base pairs in length, with a Tₘ of 72-73° C. and a percent GC content of 45-50). Both sense and antisense versions of each pathogen-specific oligonucleotide probe were prepared. Primers with sequences that flank those of the target pathogen-specific genomic sequences were also prepared (each about 23 base pairs in length, with a Tₘ of 55° C.). All primers and probes were synthesized by Qiagen Operon (Alameda, Calif.) and were dissolved in DEPC treated H₂O at a concentration of 1 μg/μl.

Primate Nucleic Acid Samples

Primate RNA samples were from the U.S. Army Medical Research Institute of Infectious Diseases, Fort Detrick, Md. Total RNA was extracted from the blood of primates infected with Ebola Zaire using TRIzol reagent (Invitrogen, Carlsbad, Calif.) according to the manufacturer's instructions.

RT-PCR Amplification

RT-PCR amplification was performed in a 25 μl volume containing 5 μl of 5× OneStep RT-PCR buffer (Qiagen, Valencia, Calif.), 1 μl of OneStep RT-PCR enzyme (Qiagen, Valencia, Calif.), 5U of RNase inhibitor (Qiagen, Valencia, Calif.), 200 μM of each of dATP, dGTP and dCTP, 120 μM of dTTP, 60 μM of amino-allyl dUTP (Sigma, St. Louis, Mo.), 0.20 to 0.50 μM of each forward primer (SEQ ID NOs: 249-344; Table 5), 0.20 to 0.50 μM of each reverse primer (SEQ ID NOs: 345-440; Table 5), sterile double-distilled water, and 1 μl of extracted (template) RNA, containing between 100 ng and 1 pg of RNA.

The RT-PCR thermocycling program consisted of one cycle of 30 minutes at 50° C. (reverse transcription); one cycle of 15 minutes at 95° C.; forty cycles of 94° C. for 30 seconds, 55° C. for 30 seconds, and 72° C. for 15 seconds (amplification); and one cycle of 15 seconds at 72° C. (final extension).

TABLE 5

| | | | |
|---|---|---|---|
| | | | SEQ |
| Gene | | | ID |
| Name | Direction | Primer | NO: |

Variola major

| | | | |
|---|---|---|---|
| VAR1S | sense | AGACAAGACGTCGGGACCAATTAC | 249 |
| VAR2S | sense | ATGAGGATGCCGAGTATGGA | 250 |
| VAR3S | sense | CCGCTAATGGAAACGCAGTGAATG | 251 |
| VAR4S | sense | GCGTTGAACGAGTGCAAGTAGAAC | 252 |
| VAR5S | sense | TTGCGAGAAGGGATCGTTGGATAC | 253 |
| VAR6S | sense | TAAATCAACCGCCAATGATGCG | 254 |
| VAR7S | sense | TATTTGAAATCGCGACTCCGGGAC | 255 |
| VAR8S | sense | AAATCAACCGCCAATGATGCGG | 256 |
| VAR9S | sense | TTGAGCGGGTCATCTGGTTTAGG | 257 |
| VAR10S | sense | AGATTGCTCTTTCAGTGGCTGGT | 258 |

Vaccinia virus

| | | | |
|---|---|---|---|
| VAC1S | sense | ATCGCGACTCCGGAACCAATTA | 259 |
| VAC2S | sense | AAATCGCGACTCCGGAACCAAT | 260 |
| VAC3S | sense | GACGAGACTCCGGAACCAATTACT | 261 |
| VAC4S | sense | GCCATTCTTCCCATGGATGTTTCC | 262 |
| VAC5S | sense | CAAACGCGGTGACATGTGTGAT | 263 |
| VAC6S | sense | GGCATCAAGACGTGGCAAACAA | 264 |
| VAC7S | sense | GGAAGAGTATTGTACGGGACTATGCG | 265 |
| VAC8S | sense | ATCTCCAGTTGAACCGAACACCTC | 266 |
| VAC9S | sense | AGACGATGTGAGAAGACTGAAGAGGA | 267 |
| VAC10S | sense | CATTGACTGCTAGAGATGCCGGT | 268 |

Ebola virus

| | | | |
|---|---|---|---|
| NPS | sense | ACTATCGGCAATTGCACTCGGA | 269 |
| VP35S | sense | ACAGGGTTTGTGCTGAGATGGT | 270 |
| VP40S | sense | CAGGCAGTGTGTCATCAGCATT | 271 |
| GP-1S | sense | CGCTGGCAACAACAACACTCAT | 272 |
| GP-2S | sense | TGCCTTCCATAAAGAGGGTGCT | 273 |
| VP30S | sense | ACTCTATGTGCTGTGATGACGAGG | 274 |
| VP24S | sense | TGTGGGCATTGAGAGTCATCCT | 275 |
| LS | sense | TCTTCAAGGGACCCTGGCTAGTAT | 276 |
| VP30-1S | sense | GTTCGAGCACGATCATCATCCAGA | 277 |
| NP-VPS | sense | AGTCAAAGAGAGTGCCAGAGCA | 278 |
| M-VP24S | sense | GCCTTATCCGACTCGCAATG | 279 |

Marburg virus

| | | | |
|---|---|---|---|
| M-LS | sense | TGCCGTATGACTGCAAGGAACT | 280 |
| M-GPS | sense | GGCCCTGGAATCGAAGGACTTTAT | 281 |
| M-VP35S | sense | GGACTACAATGCAGCCCTTGTCTA | 282 |
| M-VP40S | sense | CTGCCTCTCGGGATTATGAGCAAT | 283 |

Bacillus anthracis

| | | | |
|---|---|---|---|
| CapBS | sense | GCAACCGATTAAGCGCCGTAAA | 284 |
| CapCS | sense | TTGCGGCAACGCTAATTACAGG | 285 |
| CapA1S | sense | GTAGCAGGAGCTATTGCAACGA | 286 |
| CapA2S | sense | TGACTATGTGGGTGCTGGTGAA | 287 |
| vrrAS | sense | GCAGCAACTACAGCAGCATCAA | 288 |
| pagS | sense | GGAAATGCAGAAGTGCATGCGT | 289 |
| lef1S | sense | AGCAACCCTAGGTGCGGATTTA | 290 |
| lef2S | sense | CAGCAATTACTTTGAGTGGTCCCG | 291 |
| cyaS | sense | TTGCACCTGACCATAGAACGGT | 292 |
| lef3S | sense | ACCCTAGGTGCGGATTTAGTTGA | 293 |

Clostridium botulinum

| | | | |
|---|---|---|---|
| bont/aS | sense | CGCGAAATGGTTATGGCTCTAC | 294 |
| bont/bS | sense | AGCTACTAATGCGTCACAGGCA | 295 |
| bont/eS | sense | AGACAGGTTCTTAACTGAAAGTTCT | 296 |
| bont/fS | sense | TCGGCTATCATAAGAGGTGCTTGC | 297 |

Francisella tularensis

| | | | |
|---|---|---|---|
| TUL41S | sense | TCTAGGGTTAGGTGGCTCTGATGA | 298 |
| 16sS | sense | GGAATTACTGGGCGTAAAGGGTCT | 299 |
| TUL42S | sense | ACGCAAGCTACTGCTAAGCAAAC | 300 |
| tetCS | sense | GGATATCGTCCATTCCGACAGCAT | 301 |
| repAS | sense | TTAGCATACCCTGCTTGTTCGCC | 302 |

Lassa Fever virus

| | | | |
|---|---|---|---|
| gpc1S | sense | AGTATGAGGCAATGAGCTGCGA | 303 |
| gpc2S | sense | TTTGCAACGTGTGGCCTTGTTG | 304 |
| L-NPS | sense | CCGAAACTAAATAGCGCTGGTTCC | 305 |
| gpS | sense | CAGGAAAGGGAAACTGGGACTGTA | 306 |
| gpc3S | sense | TATGACCATGCCTCTCTCTTGCAC | 307 |

Lymphocytic Choriomeningitis virus

| | | | |
|---|---|---|---|
| LC-CS | sense | AGCAGGACTCCAAGTATTCACACG | 308 |
| LC-NPS | sense | TGGTCTTAAGCTGTCAAGGCTCTG | 309 |
| LC-GNC1S | sense | ACCTCAGTATCAGAGGGAACTCCA | 310 |
| LC-GNC2S | sense | GTACAACGTCATTGAGCGGAGTCT | 311 |

Junin virus

| | | | |
|---|---|---|---|
| SLS | sense | AAGTGCTGGGCTCATAGTTGGT | 312 |
| PLMGS | sense | CCAGTGATGACCAGGTTAGCCTTA | 313 |

Machupo virus

| | | | |
|---|---|---|---|
| MPLMG1S | sense | CCTCTAGTGACGATCAGATCACTCTT | 314 |
| MPLMG2S | sense | TCTATGTGGGAGGTGAGAGACTGT | 315 |

Guanarito virus

| | | | |
|---|---|---|---|
| GV1S | sense | TATTGAAGGCCCGCCAACTGAT | 316 |
| GV2S | sense | GGGAACAGTCCAGAATCTTTGAGC | 317 |
| GSSS | sense | TCTGTTCGTCCAGTCCAACCATAC | 318 |
| GNP-S | sense | AACAAGGAGGCTAACTCCACGAAG | 319 |

Crimean-Congo Hemorrhagic Fever virus

| | | | |
|---|---|---|---|
| CCVS | sense | TGAGATGGGTGTCTGCTTTGGA | 320 |

Hantavirus

| | | | |
|---|---|---|---|
| MSS1S | sense | ACCTCAATCAACAAGCCCAGGT | 321 |
| MSS2S | sense | ATTGGTACGGGCTGTACTGCAT | 322 |
| G1/G2S | sense | AGCCATCTGCTATGGTGCAGAA | 323 |
| MSPG-S | sense | GGCTGCAAGTGCATCAGAGAATGT | 324 |

Rift Valley Fever virus

| | | | |
|---|---|---|---|
| LG2-1S | sense | ATGGGTCAGGGATTGTGCAGAT | 325 |
| LG2-2S | sense | ACTCAGCACTGCACATGAGGTT | 326 |

Dengue virus

| | | | |
|---|---|---|---|
| NCR1S | sense | TGTGGGTAGGGCTAGAGTATCACA | 327 |
| NCR2S | sense | TCCTGGTGGTAAGGACTAGAGGTT | 328 |
| NCR3S | sense | AAGAGGAGATCTACCTGTCTGGCT | 329 |
| NCR4S | sense | TTACCAAATTCCCTGGAGGAGCTG | 330 |
| NCR5S | sense | TCCTGCGCAAACTGTGCATT | 331 |

Yersinia pestis

| | | | |
|---|---|---|---|
| rpoB1S | sense | GTGCGTTGGAAATCGAAGAGATGC | 332 |
| rpoB2S | sense | CTGTACAACGCACGTATCATCCCT | 333 |

West Nile virus

| | | | |
|---|---|---|---|
| WNV1S | sense | TGTACTTCCACAGAAGAGACCTGC | 334 |
| WNV2S | sense | CCATTTCTTCCGTAGCTTCCCTGA | 335 |
| WNV3S | sense | CTTCGCCACATCACTACACTTCCT | 336 |

SARS-CoV

| | | | |
|---|---|---|---|
| SGPS | sense | ACCACCTTATGTCCTTCCCACAAG | 337 |
| ORF 1aS | sense | GTAGCAGAGGCTGTTGTGAAGACT | 338 |
| SMPS | sense | ATTAATTGGGTGACTGGCGGGA | 339 |
| NP1S | sense | ACTTCCCTACGGCGCTAACAAA | 340 |
| EPS | sense | CATTCGTTTCGGAAGAAACAGGTACG | 341 |
| SSS | sense | CTTGTTAAAGACCCACCGAATGTGC | 342 |
| SARs1S | sense | CACCTACACACCTCAGCGTTGATA | 343 |
| SARs2S | sense | AGCTAACGAGTGTGCGCAAGTA | 344 |

Variola major

| | | | |
|---|---|---|---|
| VAR1A | antisense | GTTGGCGACACAGTAGATGGTTCA | 345 |
| VAR2A | antisense | GCACATATGCTCTCGTATCCGACT | 346 |
| VAR3A | antisense | GTCGCTGTCTTTCTCTTCTTCGCT | 347 |
| VAR4A | antisense | GCGATATACGCGACTGTTCCCTTT | 348 |
| VAR5A | antisense | GAAGCTCTTCGCCGCGACTTT | 349 |
| VAR6A | antisense | TTGGCGACACAGTAGATGGTTCAT | 350 |
| VAR7A | antisense | AATTGGTCCCGACGTCTTGTCT | 351 |
| VAR8A | antisense | AAATTGCCACGGCCGACAA | 352 |
| VAR9A | antisense | CCCTTCCAGATTGCCTCTCTGTT | 353 |
| VAR10A | antisense | AACGTAGTAGCTATAGCCGCGTCTCC | 354 |

Vaccinia virus

| | | | |
|---|---|---|---|
| VAC1A | antisense | AATTGGTTCCGGAGTCTCGTCT | 355 |
| VAC2A | antisense | AATTGGTTCCGGAGTCTCGTCT | 356 |
| VAC3A | antisense | GATCCGCATCATCGGTGGTTGATT | 357 |
| VAC4A | antisense | AACTCGGAAGCTCGTCATGTAGAC | 358 |
| VAC5A | antisense | TTCGAGAAACCCTTCTGTGGCT | 359 |
| VAC6A | antisense | ACGGATGGTCGTCGTATTCAGT | 360 |
| VAC7A | antisense | ATCACACATGTCACCGCGTTTG | 361 |
| VAC8A | antisense | ATCTCCAGTTGAACCGAACACCTC | 362 |
| VAC9A | antisense | TCGGTGTTTCGTATATCCCTGAATCC | 363 |
| VAC10A | antisense | TCAGTGTCATTTGTAGGCGATGTCA | 364 |

Ebola virus

| | | | |
|---|---|---|---|
| NPA | antisense | TCAAGTTCGCGAGACTCTGCAT | 365 |
| VP35A | antisense | AACACTTTGAGGGACGGTCTCA | 366 |
| VP40A | antisense | TATGAAGCAAGCATGATGGCGG | 367 |
| GP-1A | antisense | GGGTTGCATTTGGGTTGAGCAT | 368 |
| GP-2A | antisense | CAGAAATGCAACGACACCTTCAGC | 369 |
| VP30A | antisense | TCGGTCCCATTGTTGCCATAGT | 370 |
| VP24A | antisense | CCTTGACACGTTGTGTTCGCAT | 371 |
| LA | antisense | AATCCAGAGGTTTGCCGAGTGT | 372 |
| VP30-1A | antisense | GTCTTTAGGTGCTGGAGGAACTGT | 373 |
| NP-VPA | antisense | TAGCAAGGCTTCTGCGAGTGTT | 374 |
| M-VP24A | antisense | CACTTGTGTGGTGCCATGATGC | 375 |

Marburg virus

| | | | |
|---|---|---|---|
| M-LA | antisense | GCCAGACGATTAACAGAGGGATGT | 376 |
| M-GPA | antisense | GTCCTTTCCTCGGTTGTGACTCTT | 377 |
| M-VP35A | antisense | GCCGCTCAATTTCATGGACTCTTG | 378 |
| M-VP40A | antisense | CCGAGTCGATTTACACGGACGAAT | 379 |

Bacillus anthracis

| | | | |
|---|---|---|---|
| CapBA | antisense | CGGGTTGAACTGCCATACATTCAC | 380 |
| CapCA | antisense | CCTGGAACAATAACTCCAATACCACGG | 381 |
| CapA1A | antisense | TGTACGTTGTACCCATGTCGCA | 382 |
| CapA2A | antisense | CGAACCTGGTTGTTCTTTCGTTGC | 383 |
| VrrAA | antisense | ACGATGCTTGGTAGGTTACGCA | 384 |
| PagA | antisense | ACACGTTGTAGATTGGAGCCGT | 385 |
| lef1A | antisense | CCTGCTCGAGTATCTGGTGA | 386 |
| lef2A | antisense | TGCATACCTACATCACCATGACCG | 387 |
| cyaA | antisense | TCCCTTTGTAGCCACACCACTT | 388 |
| lef3A | antisense | TCCTGCTCGAGTATCTGGTGAT | 389 |

Clostridium botulinum

| | | | |
|---|---|---|---|
| bont/aA | antisense | CCTCAAAGCTTACTTCTAACCCACTCA | 390 |
| bont/bA | antisense | TTCCCATGAGCAACCCAAAGTC | 391 |
| bont/eA | antisense | AGTTCTTGCTGACTCTCTCCCAAG | 392 |
| bont/fA | antisense | TTCTTGTATTGGGAGCAGGACCTG | 393 |

Francisella tularensis

| | | | |
|---|---|---|---|
| TUL41A | antisense | AGCAGCTTGCTCAGTAGTAGCTGT | 394 |
| 16sA | antisense | TACGCATTTCACCGCTACACCA | 395 |
| TUL42A | antisense | ACCTTCTGGAGCTTGCCATTGT | 396 |
| tetCA | antisense | GGGTGCGCATAGAAATTGCATC | 397 |
| repAA | antisense | TACGCATTTCACCGCTACACCA | 398 |

Lassa Fever virus

| | | | |
|---|---|---|---|
| gpc1A | antisense | TGAGTCAAGAGCAATGTAGCTCCC | 399 |
| gpc2A | antisense | GCAGGAGAGAGGCATGGTCATATT | 400 |
| L-NPA | antisense | GGCCTGCCCTCAATATCTATCCAT | 401 |
| gpA | antisense | TCTGACAATGTCCAGGTGAAGGTC | 402 |
| gpc3A | antisense | TGGTGTTGGTCAAGGTCAGTTCT | 403 |

Lymphocytic Choriomeningitis virus

| | | | |
|---|---|---|---|
| LC-CA | antisense | TCAGAACCTTGACAGCTCAAGACC | 404 |
| LC-NPA | antisense | CAGTGTGCATCTTGCATAACCAGC | 405 |
| LC-GNC1A | antisense | GTTCTACACTGGCTCTGAGCACTT | 406 |
| LC-GNC2A | antisense | TAGTTGGGATGAGAAAGCCTCAGC | 407 |

Junin virus

| | | | |
|---|---|---|---|
| SLA | antisense | ATGTGGGAGACCTCAACACTAAGC | 408 |
| PLMGA | antisense | GGTTCCTATCACACTCTTTGGGCT | 409 |

Machupo virus

| | | | |
|---|---|---|---|
| MPLMG1A | antisense | CGATGACACTCTTGGGACTGACAA | 410 |
| MPLMG2A | antisense | CAAATAAGGCCACAGTTGGTGCAG | 411 |

Guanarito virus

| | | | |
|---|---|---|---|
| GV1A | antisense | AATGCCGTGTGAGTGCCTACTT | 412 |
| GV2A | antisense | CGTGGAGTTGGCTTCCTTGTTT | 413 |
| GSSA | antisense | CACAGCAGATTCTTGGATCCCTCA | 414 |
| GNPA | antisense | TGAATGGGAATGGTGTCGGGAA | 415 |

Crimean-Congo Hemorrhagic Fever virus

| | | | |
|---|---|---|---|
| CCVA | antisense | CTTGGCACACGGATTGTTGGTT | 416 |

Hantavirus

| | | | |
|---|---|---|---|
| MSS1A | antisense | TGCATTGTCATTCCAGCTTGGC | 417 |
| MSS2A | antisense | ACGGCTGTAACGGACTGTGATT | 418 |
| G1/G2A | antisense | AACCACCCTTCCCTGACACTTT | 419 |
| MSPG-A | antisense | CCCAGATCAGTATGCATTGGGACA | 420 |

Rift Valley Fever virus

| | | | |
|---|---|---|---|
| LG2-1A | antisense | TGCAAGGCTCAACTCTCTGGAT | 421 |
| LG2-2A | antisense | AGTCTGACCAGAGTCCATTGTTCC | 422 |

Dengue virus

| | | | |
|---|---|---|---|
| NCR1A | antisense | TCAGGTCTCTCCTGTGGAAGTACA | 423 |
| NCR2A | antisense | TGCCTGGAATGATGCTGTAGAGAC | 424 |
| NCR3A | antisense | TTTGTCCACATCTCCACGTCCA | 425 |
| NCR4A | antisense | AAGAGATCCCAATAGCACCGGAAG | 426 |
| NCR5A | antisense | TTCGCGTCTTGTTCTTCCACCA | 427 |

Yersinia pestis

| | | | |
|---|---|---|---|
| rpoB1A | antisense | TCGATGCCACCAGAAACCAGAA | 428 |
| rpoB2A | antisense | GCAATTTACGGCGACGGTCAATAC | 429 |

West Nile virus

| | | | |
|---|---|---|---|
| WNV1A | antisense | AAACACGGTTCCAGACCTCCAA | 430 |
| WNV2A | antisense | CCACCACGATGTAAGAGTCACCAA | 431 |
| WNV3A | antisense | CGTCCTTCATGATCAGTTCCGTGA | 432 |

SARS-CoV

| | | | |
|---|---|---|---|
| SGPA | antisense | TCATGACAAATTGCTGGCGCTG | 433 |
| ORF 1aA | antisense | CACACTCTGCATCGTCCTCTTCTT | 434 |
| SMPA | antisense | GCAAACAGCCTGAAGGAAGCAA | 435 |
| NP1A | antisense | GCACGGTGGCAGCATTGTTATT | 436 |
| EPA | antisense | ATTGCAGCAGTACGCACACAATC | 437 |
| SSA | antisense | TAGTAGTCGTCGTCGGCTCATCATA | 438 |
| SARs1A | antisense | CGAATAGCTTCTTCGCGGGTGATA | 439 |
| SARs2A | antisense | AAGCAGTTGTAGCATCACCGGA | 440 |

Purification of RT-PCR Products and Dye Conjugation

Following the RT-PCR, amplified pathogen-specific genomic sequences were purified with the QiaQuick PCR Purification Kit (Qiagen, Valencia, Calif.) according to the manufacturer's instructions, vacuum-dried and eluted in 9 μl of sterile double-distilled water. To the eluate, 1 μl of 1M sodium bicarbonate buffer pH 9.0 was added, followed by 4.5 μl NHS-cye dye (Cy3 or Cy5, Pharmacia, Piscataway, N.J.). The conjugation mixture was then incubated at room-temperature for one hour in the dark and quenched with 4 M hydroxylamine.

To remove unincorporated cye dyes, the conjugation mixture was purified with the QiaQuick PCR Purification Kit (Qiagen, Valencia, Calif.) according to the manufacturer's instructions, and Cy3- and Cy5-labeled products were combined. To the combined Cy3- and Cy5-labeled products, 60 μl of sterile double-distilled water was added, followed by 500 μl of PB buffer (Qiagen, Valencia, Calif.). The mixture was applied to a QiaQuick column and spun at 13,000 rpm for 1 minute. The flow-through was reloaded onto the same column for a second spin and then discarded. The column was washed twice with 500 μl of PE buffer (Qiagen, Valencia, Calif.) and spun at 13,000 rpm for 1 minute. Dye-conjugated amplified virus-specific genomic sequences were eluted from the column with 20 μl of EB buffer (Qiagen, Valencia, Calif.) for 1 minute room-temperature, followed by centrifugation at 13,000 rpm for 1 minute. The elution step was repeated two additional times.

Production of Oligonucleotide Microarrays

Oligonucleotide probes (SEQ ID NOs: 441-632; Table 6) were solubilized in 50% DMSO and spotted in duplicate on poly-L-lysine coated slides or Ultra GAPS slides (Corning, Acton, Mass.) using an OmniGrid arrayer (Gene Machines, San Carlos, Calif.) at a concentration of 50 μM. Slides were processed for hybridization according to Xiang and Brownstein (Fabrication of cDNA microarrays, in: Methods in Molecular Biology, vol. 224, Functional Genomics, Methods and Protocols, M. J. Brownstein and A. Khodursky (eds.), Humana Press Inc., 2003).

TABLE 6

| | | | |
|---|---|---|---|
| | | | SEQ |
| Gene | Direction & | | ID |
| Name | Position | Oligonucleotide | NO: |

Variola major

| | | | |
|---|---|---|---|
| VAR1S | sense/A1 | CAACCGCCAATGATGCGCACAATGATAATGAACCATCTACTGTGTCGCCAACAACTG | 441 |
| VAR2S | sense/A2 | ATCTCTCATCTGTATTCAGAGTCGGATACGAGAGCATATGTGCGTCCGGAAGTTGTT | 442 |
| VAR3S | sense/A3 | AGGTAAGGACTCTCCCGCTATCACTCGTGTAGAAGCTCTGGCTATGATCAAAGACTG | 443 |
| VAR4S | sense/A4 | CAAATCTCGCGTTGAACGAGTGCAAGTAGAACTTACTGACAAAGTTAAGGTGCGAGT | 444 |
| VAR5S | sense/A5 | GGCATTAGAACCTAATTACGACGTAGAAAGTCGCGGCGAAGAGCTTCCGCTATCTAC | 445 |
| VAR6S | sense/A6 | CAACCGCCAATGATGCGCACAATGATAATGAACCATCTACTGTGTCGCCAACAACTGTA | 446 |
| VAR7S | sense/A7 | ACAGTAAGTACATCATCTGGAGAATCCACAACAGACAAGACGTCGGGACCAATTACT | 447 |
| VAR8S | sense/A8 | TGCGGATCTTTATGATACGCACAATGATAATGAACCATCTACTGTGTCACCAACAACTGT | 448 |
| VAR9S | sense/A9 | GGATTTGTGGTGTCCCATACCACTAGATTTCCTCGTCCTATGGAACGAGAAGGTG | 449 |
| VAR10S | sense/A10 | CTACATTCCCGGGAGACGCGGCTATAGCTACTACGTTTACGGTATAGCCTCTAG | 450 |

Vaccinia virus

| | | | |
|---|---|---|---|
| VAC1S | sense/A11 | ACAGTAAGTGCATCATCTGGAGAATCCACAACAGACGAGACTCCGGAACCAATT | 451 |
| VAC2S | sense/A12 | TGCTCGTCGGTATTCGAAATCGCGACTCCGGAACCAATTACTGATAATGTAGAAGATCA | 452 |
| VAC3S | sense/B1 | ACACTACAGTAAGTACATCATCTGGAATTGTCACTACTAAATCAACCACCGATGATGCGGA | 453 |
| VAC4S | sense/B2 | ATTCTCCTGTTTGATTTCTCTATCGATGCGGCACCTCTCTTAAGAAGTGTAACCGAT | 454 |
| VAC5S | sense/B3 | TCGATGACTACGATTGCACGTCTACAGGTTGCAGCATAGACTTTGTCACAACAGAA | 455 |
| VAC6S | sense/B4 | TTGAACAAGGCGATTATAAAGTGGAAGAGTATTGTACGGGACCACCGACTGTAACA | 456 |
| VAC7S | sense/B5 | GGACCACCGACTGTAACATTAACTGAATACGACGACCATATCAATTTGTACATCGAGCATCCG | 457 |
| VAC8S | sense/B6 | CTGGTGGATACTCTAGTAAAGTCAGGACTGACAGAGGTGTTCGGTTCAACTGGAGA | 458 |
| VAC9S | sense/B7 | CTATCCCGAAAGAACTGATTGCAGTGTTCATCTCCCAACTGCAAGTGAAGGATTGA | 459 |
| VAC10S | sense/B8 | TCATTGACTGCTAGAGATGCCGGTACTTATGTATGTGCATTCTTTATGACATCGCCTACA | 460 |

Ebola virus

| | | | |
|---|---|---|---|
| NPS | sense/B9 | GGAGTAAATGTTGGAGAACAGTATCAACAACTCAGAGAGGCTGCCACTGAGGCTG | 461 |
| VP35S | sense/B10 | CCGAACATGGTCAACCACCACCTGGACCATCACTTTATGAAGAAAGTGCGATTCG | 462 |
| VP40S | sense/B11 | GACCTACAGCTTTGACTCAACTACGGCCGCCATCATGCTTGCTTCATACACTATC | 463 |
| GP-1S | sense/B12 | GAAGAGAGTGCCAGCAGCGGGAAGCTAGGCTTAATTACCAATACTATTGCTGGAG | 464 |
| GP-2S | sense/C1 | GATCGACTTGCTTCCACAGTTATCTACCGAGGAACGACTTTCGCTGAAGGTGTC | 465 |
| VP30S | sense/C2 | GGCTTGGGCAAGATCAGGCAGAACCCGTTCTCGAAGTATATCAACGATTACACAG | 466 |
| VP24S | sense/C3 | CTGATTGACCAGTCTTTGATTGAACCCTTAGCAGGAGCCCTTGGTCTGATCTCTG | 467 |
| LS | sense/C4 | GCGATCCATCTCTGAGACACGACATATCTTTCCTTGCAGGATAACCGCAGCTTTC | 468 |
| VP30-1S | sense/C5 | CCGTCAATCAAGGAGCGCCTCACAAGTGCGCGTTCCTACTGTATTTCATAAGAAGAGAG | 469 |
| NP-VPS | sense/C6 | ATCGTGTCAGAAATGTCCAAACACTCGCAGAAGCCTTGCTAGCAGATGGACTAG | 470 |
| M-VP24S | sense/C7 | CCGACTCGCAATGTTCAAACACTTTGTGAAGCTCTGTTAGCTGATGGTCTTGCT | 471 |

Marburg virus

| | | | |
|---|---|---|---|
| M-LS | sense/C8 | GCGACTTGGAAGAAGCAATGACACAGAGTTAAACTATGTCAGTTGTGCTCTCGACCGG | 472 |
| M-GPS | sense/C9 | GGTCTGCAGGTTGAGGCGTCTAGCCAATCAAACTGCCAAATCCTTGGAACTCTTATT | 473 |
| M-VP35S | sense/C10 | ATGTCAAAGGCGACAAGCACTGATGATATTGTTTGGGACCAACTGATCGTGAAGAAA | 474 |
| M-VP40S | sense/C11 | CCTTTAGCTCATACTGTGGCTGCGTTGCTCACAGGCAGCTATACAATCACCCAATTTAC | 475 |

Bacillus anthracis

| | | | |
|---|---|---|---|
| CapBS | sense/C12 | CGTAAAGAAGGTCCTAATATCGGTGAGCAACGCAGGGTAGTTAAAGAGGCTGCTG | 476 |
| CapCS | sense/D1 | CCGTGGTATTGGAGTTATTGTTCCAGGATTAATTGCAAATACAATTCAAAGACAAGGG | 477 |
| CapA1S | sense/D2 | CGCAGTTATATTAATAGCTGCGACATGGGTACAACGTACAGAAGCAGTAGCACCAGT | 478 |
| CapA2S | sense/D3 | GGTGTTAGGGTTGCTACTCTTGGATTTACAGATGCATTTGTAGCAGGAGCTATTGCAACG | 479 |
| vrrAS | sense/D4 | GTCGTTCAATCTGTAAGCCCTGTCGTCGAACAGTACGGTCCCATTATGCGTAAC | 480 |
| pagS | sense/D5 | ACTGGGACGGCTCCAATCTACAACGTGTTACCAACGACTTCGTTAGTGT | 481 |
| lef1S | sense/D6 | GGCCTGCATTAGATAATGAGCGTTTGAAATGGAGAATCCAATTATCACCAGATACTCGAGC | 482 |
| lef2S | sense/D7 | GGGCGGGCGGTCATGGTGATGTAGGTATGCACGTAAA | 483 |
| cyaS | sense/D8 | AAGTGGTGTGGCTACAAAGGGATTGAATGAACATGGAAAGAGTTCGGATTGGG | 484 |
| lef3S | sense/D9 | GGCCTGCATTAGATAATGAGCGTTTGAAATGGAGAATCCAATTATCACCAGATACTCGAGC | 485 |

Clostridium botulinum

| | | | |
|---|---|---|---|
| bont/aS | sense/D10 | GGTGCAGGCAAATTTGCTACAGATCCAGCAGTAACATTAGCACATGAACTTATACATGCTGG | 486 |
| bont/bS | sense/D11 | TCTAGTAGGACTTTGGGTTGCTCATGGGAATTTATTCCTGTAGATGATGGATGGG | 487 |
| bont/eS | sense/D12 | TGGATCAATCTTGGGAGAGAGTCAGCAAGAACTAAATTCTATGGTAACTGATACCCT | 488 |
| bont/fS | sense/E1 | ATTGCAGATCCTGCAATTTCACTAGCTCATGAATTGATACATGCACTGCATGGA | 489 |

Francisella tularensis

| | | | |
|---|---|---|---|
| TUL41S | sense/E2 | CTTCAGCTAAAGATACTGCTGCTGCTCAGACAGCTACTACTGAGCAAGCTGCTG | 490 |
| 16sS | sense/E3 | CAGGGCTCAACCTTGGAACTGCATTTGATACTGGCAAACTAGAGTACGGTAGAGG | 491 |
| TUL42S | sense/E4 | GCTACGCAAGCTACTGCTAAGCAAACAGGTGTATCTAAGCCAACTGCAAAGGT | 492 |
| tetCS | sense/E5 | CCAGTCACTATGGCGTGCTGCTAGCGCTATATGCGTTGATGCAATTTCTATGCG | 493 |
| repAS | sense/E6 | GCAAATTAGGCGAACAAGCAGGGTATGCTAATATAGTTGATTGCGTATTGTATGTCGA | 494 |

Lassa Fever virus

| | | | |
|---|---|---|---|
| gpc1S | sense/E7 | GAGCTACATTGCTCTTGACTCAGGCCGTGGCAACTGGGACTGTATTATGACTAG | 495 |
| gpc2S | sense/E8 | CTTGTTGGTTTGGTCACTTTCCTCCTGTTGTGTGGTAGGTCTTGCACAACCAGTC | 496 |
| L-NPS | sense/E9 | GCTTGCAGGCTGCAGGTCTAAATGCTGGGTTGACCTATTCTCAACTGATGACAC | 497 |
| gpS | sense/E10 | AAATACAACATGGGAGGACCACTGCCAATTCTCAAGACCGTCTCCTATCGGGTAC | 498 |
| gpc3S | sense/E11 | TACATAAGGGTGGGCAATGAGACAGGACTAGAACTGACCTTGACCAACACCAGTA | 499 |

Lymphocytic Choriomeningitis virus

| | | | |
|---|---|---|---|
| LC-CS | sense/E12 | ATGGATCTTGCTGACCTCTTCAATGCACAGGCTGGGCTGACCTCATCAGTTATAG | 500 |
| LC-NPS | sense/F1 | GATGTTGAGATGACCAAAGAGGCTTCAAGAGAGTATGAAGACAAAGTGTGGGACA | 501 |
| LC-GNC1S | sense/F2 | GGCAGTATCCTGCGACTTCAACAATGGCATAACCATCCAATACAACTTGACATTC | 502 |
| LC-GNC2S | sense/F3 | CGTCATTGAGCGGAGTCTGTGACTGTTTGGCCATACAAGCCATAGTTAGACTTGG | 503 |

Junin virus

| | | | |
|---|---|---|---|
| SLS | sense/F4 | CTCATTGAACTACCCACAGCTTCTGAGAAGTCTTCAACTAACCTGGTCATCAGCT | 504 |
| PLMGS | sense/E5 | AGTGTCAAGACAGAACAGAACTGCTGGAAATGGTGTGCTTCCATGAATTCTTATCA | 505 |

Machupo virus

| | | | |
|---|---|---|---|
| MPLMG1S | sense/F6 | CAGATGCTGCGGAGTGGCTCGAGATGATCTGCTTCCATGAGTTTCTGTCATCTAA | 506 |
| MPLMG2S | sense/F7 | GGGCCCTAGAAGATGATGAGAGTGTTGTTTCTATGCTGCACCAACTGTGGCCTTA | 507 |

Guanarito virus

| | | | |
|---|---|---|---|
| GV1S | sense/F8 | CCGTGGAGTTGGCAGTGTTTCAGCCTTCTTCAGGAAACTATGTACACTGCTTCAG | 508 |
| GV2S | sense/F9 | AAATTTGGCCATCTCTGCAGAGCACACAATGGTGTCATTGTTCCCAAGAAGAA | 509 |
| GSSS | sense/F10 | GGCTCTCCAGAGTTTGATTTGGATTCTTGGGTGGACAATTAAGGGATTGGGACATG | 510 |
| GNP-S | sense/F11 | CTAACTCCACGAAGGAGCCACACTGTGCTCTTCTCGATTGCATCATGTTTCAGTC | 511 |

Crimean-Congo Hemorrhagic Fever virus

| | | | |
|---|---|---|---|
| CCVS | sense/F12 | GGGATCTGGACACACCAAGTCCATTCTTAACCTACGGACAAACACCGAAACCAAC | 512 |

Hantavirus

| | | | |
|---|---|---|---|
| MSS1S | sense/G1 | GAATCAATCATGTGGGCAGCTAGTGCATCAGAAACTGTCTTGGAGCCAAGCTGG | 513 |
| MSS2S | sense/G2 | GTACGGGCTGTACTGCATGCGGACTATACATTGACCAACTTAAACCTGTAGGCAG | 514 |
| G1/G2S | sense/G3 | CAAGAGGCCAGAATACAGTCAAAGTGTCAGGGAAGGGTGGTTATAGTGGCTCAAC | 515 |
| MSPG-S | sense/G4 | CCAAGCTGGAATGACAACGCACATGGTGTTGGTGTTGTCCCAATGCATACTGATC | 516 |

Rift Valley Fever virus

| | | | |
|---|---|---|---|
| LG2-1S | sense/G5 | GCCTTTATGTGTAGGGTATGAGAGAGTGGTTGTGAAGAGAGAACTCTCTGCCAAGCC | 517 |
| LG2-2S | sense/G6 | CTCAGCACTGCACATGAGGTTGTGCCCTTTGCAGTGTTTAAGAACTCAAAGAAGG | 518 |

Dengue virus

| | | | |
|---|---|---|---|
| NCR1S | sense/G7 | GCAGCTGATGTACTTCCACAGGAGAGACCTGAGACTAGCTGCTAATGCTATCTGT | 519 |
| NCR2S | sense/G8 | CCGGCATAACAATAAACAGCATATTGACGCTGGGAGAGACCAGAGATCCTGC | 520 |
| NCR3S | sense/G9 | CAAAGTTGCCTCAGAAGGCTTCCAGTACTCTGACAGAAGATGGTGCTTTGACGG | 521 |
| NCR4S | sense/G10 | GACTGACTTTCAGTCACATCAGCTGTGGGCTACCTTGCTGTCCTTGACATTTGTC | 522 |
| NCR5S | sense/G11 | CGATTCAAGATGTCCAACACAAGGAGAAGCTACACTGGTGGAAGAACAAGACGCG | 523 |

Yersinia pestis

| | | | |
|---|---|---|---|
| rpoB1S | sense/G12 | CAACTGAAACAGGCTAAGAAAGACCTGACTGAAGAGTTGCAGATCCTGGAAGCGG | 524 |
| rpoB2S | sense/H1 | CAACGCACGTATCATCCCTTACCGCGGTTCATGGTTAGATTTCGAGTTTGATCCG | 525 |

West Nile virus

| | | | |
|---|---|---|---|
| WNV1S | sense/H2 | GAAGAACCACGTGGTCCATCCATGCAGGAGGAGAGTGGATGACAACAGAG | 526 |
| WNV2S | sense/H3 | ATGACCTCACACCTGTTGGAAGACTGGTGACCGTGAATCCATTTGTGTCTGTG | 527 |
| WNV3S | sense/H4 | AAATGCTATGTCAAAGGTCCGCAAAGACATCCAGGAATGGAAACCCTCGACGG | 528 |

SARS-CoV

| | | | |
|---|---|---|---|
| SGPS | sense/H5 | CATGGTGTTGTCTTCCTACATGTCACGTATGTGCCATCCCAGGAGAGGAACTTCAC | 529 |
| ORF 1aS | sense/H6 | ACCAGTTTCTGATCTCCTTACCAACATGGGTATTGATCTTGATGAGTGGAGTGTAGCT | 530 |
| SMPS | sense/H7 | GTATTGTAGGCTTGATGTGGCTTAGCTACTTCGTTGCTTCCTTCAGGCTGTTTGCTCG | 531 |
| NP1S | sense/H8 | CATCGTATGGGTTGCAACTGAGGGAGCCTTGAATACACCCAAAGACCACATTGG | 532 |
| EPS | sense/H9 | TTTCTTGCTTTCGTGGTATTCTTGCTAGTCACACTAGCCATCCTTACTGCGCTTC | 533 |
| SSS | sense/H10 | AAATACACACAATCGACGGCTCTTCAGGAGTTGCTAATCCAGCAATGGATCCAATT | 534 |
| SARs1S | sense/H11 | TGACATACCAGGCATACCAAAGGACATGACCTACCGTAGACTCATCTCTATGATGGGT | 535 |
| SARs2S | sense/H12 | AAGTGAGATGGTCATGTGTGGCGGCTCACTATATGTTAAACCAGGTGGAACATCA | 536 |

Variola major

| | | | |
|---|---|---|---|
| VAR1A | antisense/A1 | CAGTTGTTGGCGACACAGTAGATGGTTCATTATCATTGTGCGCATCATTGGCGGTTG | 537 |
| VAR2A | antisense/A2 | AACAACTTCCGGACGCACATATGCTCTCGTATCCGACTCTGAATACAGATGAGAGAT | 538 |
| VAR3A | antisense/A3 | CAGTCTTTGATCATAGCCAGAGCTTCTACACGAGTGATAGCGGGAGAGTCCTTACCT | 539 |
| VAR4A | antisense/A4 | ACTCGCACCTTAACTTTGTCAGTAAGTTCTACTTGCACTCGTTCAACGCGAGATTTG | 540 |
| VAR5A | antisense/A5 | GTAGATAGCGGAAGCTCTTCGCCGCGACTTTCTACGTCGTAATTAGGTTCTAATGCC | 541 |
| VAR6A | antisense/A6 | TACAGTTGTTGGCGACACAGTAGATGGTTCATTATCATTGTGCGCATCATTGGCGGTTG | 542 |
| VAR7A | antisense/A7 | AGTAATTGGTCCCGACGTCTTGTCTGTTGTGGATTCTCCAGATGATGTACTTACTGT | 543 |
| VAR8A | antisense/A8 | ACAGTTGTTGGTGACACAGTAGATGGTTCATTATCATTGTGCGTATCATAAAGATCCGCA | 544 |
| VAR9A | antisense/A9 | CACCTTCTCGTTCCATAGGACGAGGAAATCTAGTGGTATGGGACACCACAAATCC | 545 |
| VAR10A | antisense/A10 | CTAGAGGCTATACCGTAAACGTAGTAGCTATAGCCGCGTCTCCCGGGAATGTAG | 546 |

Vaccinia virus

| | | | |
|---|---|---|---|
| VAC1A | antisense/A11 | AATTGGTTCCGGAGTCTCGTCTGTTGTGGATTCTCCAGATGATGCACTTACTGT | 547 |
| VAC2A | antisense/A12 | TGATCTTCTACATTATCAGTAATTGGTTCCGGAGTCGCGATTTCGAATACCGACGAGCA | 548 |
| VAC3A | antisense/B1 | TCCGCATCATCGGTGGTTGATTTAGTAGTGACAATTCCAGATGATGTACTTACTGTAGTGT | 549 |
| VAC4A | antisense/B2 | ATCGGTTACACTTCTTAAGAGAGGTGCCGCATCGATAGAGAAATCAAACAGGAGAAT | 550 |
| VAC5A | antisense/B3 | TTCTGTTGTGACAAAGTCTATGCTGCAACCTGTAGACGTGCAATCGTAGTCATCGA | 551 |
| VAC6A | antisense/B4 | TGTTACAGTCGGTGGTCCCGTACAATACTCTTCCACTTTATAATCGCCTTGTTCAA | 552 |
| VAC7A | antisense/B5 | CGGATGCTCGATGTACAAATTGATATGGTCGTCGTATTCAGTTAATGTTACAGTCGGTGGTCC | 553 |
| VAC8A | antisense/B6 | TCTCCAGTTGAACCGAACACCTCTGTCAGTCCTGACTTTACTAGAGTATCCACCAG | 554 |
| VAC9A | antisense/B7 | TCAATCCTTCACTTGCAGTTGGGAGATGAACACTGCAATCAGTTCTTTCGGGATAG | 555 |
| VAC10A | antisense/B8 | TGTAGGCGATGTCATAAAGAATGCACATACATAAGTACCGGCATCTCTAGCAGTCAATGA | 556 |

Ebola virus

| | | | |
|---|---|---|---|
| NPA | antisense/B9 | CAGCCTCAGTGGCAGCCTCTCTGAGTTGTTGATACTGTTCTCCAACATTTACTCC | 557 |
| VP35A | antisense/B10 | CGAATCGCACTTTCTTCATAAAGTGATGGTCCAGGTGGTGGTTGACCATGTTCGG | 558 |
| VP40A | antisense/B11 | GATAGTGTATGAAGCAAGCATGATGGCGGCCGTAGTTGAGTCAAAGCTGTAGGTC | 559 |
| GP-1A | antisense/B12 | CTCCAGCAATAGTATTGGTAATTAAGCCTAGCTTCCCGCTGCTGGCACTCTCTTC | 560 |
| GP-2A | antisense/C1 | GACACCTTCAGCGAAAGTCGTTCCTCGGTAGATAACTGTGGAAGCAAGTCGATC | 561 |
| VP30A | antisense/C2 | CTGTGTAATCGTTGATATACTTCGAGAACGGGTTCTGCCTGATCTTGCCCAAGCC | 562 |
| VP24A | antisense/C3 | CAGAGATCAGACCAAGGGCTCCTGCTAAGGGTTCAATCAAAGACTGGTCAATCAG | 563 |
| LA | antisense/C4 | GAAAGCTGCGGTTATCCTGCAAGGAAAGATATGTCGTGTCTCAGAGATGGATCGC | 564 |
| VP30-1A | antisense/C5 | CTCTCTTCUATGAAATACAGTAGGAACGCGCACTTGTGAGGCGCTCCTTGATTGACGG | 565 |
| NP-VPA | antisense/C6 | CTAGTCCATCTGCTAGCAAGGCTTCTGCGAGTGTTTGGACATTTCTGACACGAT | 566 |
| M-VP24A | antisense/C7 | AGCAAGACCATCAGCTAACAGAGCTTCACAAAGTGTTTGAACATTGCGAGTCGG | 567 |

Marburg virus

| | | | |
|---|---|---|---|
| M-LA | antisense/C8 | CCGGTCGAGAGCACAACTGACATAGTTTAACTCTGTGTCATTGCTTCTTCCAAGTCGC | 568 |
| M-GPA | antisense/C9 | AATAAGAGTTCCAAGGATTTGGCAGTTTGATTGGCTAGACGCCTCAACCTGCAGACC | 569 |
| M-VP35A | antisense/C10 | TTTCTTCACGATCAGTTGGTCCCAAACAATATCATCAGTGCTTGTCGCCTTTGACAT | 570 |
| M-VP40A | antisense/C11 | GTAAATTGGGTGATTGTATAGCTGCCTGTGAGCAACGCAGCCACAGTATGAGCTAAAGG | 571 |

Bacillus anthracis

| | | | |
|---|---|---|---|
| CapBA | antisense/C12 | CAGCAGCCTCTTTAACTACCCTGCGTTGCTCACCGATATTAGGACCTTCTTTACG | 572 |
| CapCA | antisense/D1 | CCCTTGTCTTTGAATTGTATTTGCAATTAATCCTGGAACAATAACTCCAATACCACGG | 573 |
| CapA1A | antisense/D2 | ACTGGTGCTACTGCTTCTGTACGTTGTACCCATGTCGCAGCTATTAATATAACTGCG | 574 |
| CapA2A | antisense/D3 | CGTTGCAATAGCTCCTGCTACAAATGCATCTGTAAATCCAAGAGTAGCAACCCTAACACC | 575 |
| VrrAA | antisense/D4 | GTTACGCATAATGGGACCGTACTGTTCGACGACAGGGCTTACAGATTGAACGAC | 576 |
| PagA | antisense/D5 | ACACTAACGAAGTCGTTGGTAACACGTTGTAGATTGGAGCCGTCCCAGT | 577 |
| lef1A | antisense/D6 | GCTCGAGTATCTGGTGATAATTGGATTCTCCATTTCAAACGCTCATTATCTAATGCAGGCC | 578 |
| lef2A | antisense/D7 | TTTACGTGCATACCTACATCACCATGACCGCCCGCCC | 579 |
| cyaA | antisense/D8 | CCCAATCCGAACTCTTTCCATGTTCATTCAATCCCTTTGTAGCCACACCACTT | 580 |
| lef3A | antisense/D9 | GCTCGAGTATCTGGTGATAATTGGATTCTCCATTTCAAACGCTCATTATCTAATGCAGGCC | 581 |

Clostridium botulinum

| | | | |
|---|---|---|---|
| bont/aA | antisense/D10 | GGTGCAGGCAAATTTGCTACAGATCCAGCAGTAACATTAGCACATGAACTTATACATGCTGG | 582 |
| bont/bA | antisense/D11 | CCCATCCATCATCTACAGGAATAAATTCCCATGAGCAACCCAAAGTCCTACTAGA | 583 |
| bont/eA | antisense/D12 | AGGGTATCAGTTACCATAGAATTTAGTTCTTGCTGACTCTCTCCCAAGATTGATCCA | 584 |
| bont/fA | antisense/E1 | TCCATGCAGTGCATGTATCAATTCATGAGCTAGTGAAATTGCAGGATCTGCAAT | 585 |

Francisella tularensis

| | | | |
|---|---|---|---|
| TUL41A | antisense/E2 | CAGCAGCTTGCTCAGTAGTAGCTGTCTGAGCAGCAGCAGTATCTTTAGCTGAAG | 586 |
| 16sA | antisense/E3 | CCTCTACCGTACTCTAGTTTGCCAGTATCAAATGCAGTTCCAAGGTTGAGCCCTG | 587 |
| TUL42A | antisense/E4 | ACCTTTGCAGTTGGCTTAGATACACCTGTTTGCTTAGCAGTAGCTTGCGTAGC | 588 |
| tetCA | antisense/E5 | CGCATAGAAATTGCATCAACGCATATAGCGCTAGCAGCACGCCATAGTGACTGG | 589 |
| repAA | antisense/E6 | TCGACATACAATACGCAATCAACTATATTAGCATACCCTGCTTGTTCGCCTAATTTGC | 590 |

Lassa Fever virus

| | | | |
|---|---|---|---|
| gpc1A | antisense/E7 | CTAGTCATAATACAGTCCCAGTTGCCACGGCCTGAGTCAAGAGCAATGTAGCTC | 591 |
| gpc2A | antisense/E8 | GACTGGTTGTGCAAGACCTACCACACAACAGGAGGAAAGTGACCAAACCAACAAG | 592 |
| L-NPA | antisense/E9 | GTGTCATCAGTTGAGAATAGGTCAACCCAGCATTTAGACCTGCAGCCTGCAAGC | 593 |
| gpA | antisense/E10 | GTACCCGATAGGAGACGGTCTTGAGAATTGGCAGTGGTCCTCCCATGTTGTATTT | 594 |
| gpc3A | antisense/E11 | TACTGGTGTTGGTCAAGGTCAGTTCTAGTCCTGTCTCATTGCCCACCCTTATGTA | 595 |

Lymphocytic Choriomeningitis virus

| | | | |
|---|---|---|---|
| LC-CA | antisense/E12 | CTATAACTGATGAGGTCAGCCCAGCCTGTGCATTGAAGAGGTCAGCAAGATCCAT | 596 |
| LC-NPA | antisense/F1 | TGTCCCACACTTTGTCTTCATACTCTCTTGAAGCCTCTTTGGTCATCTCAACATC | 597 |
| LC-GNC1A | antisense/F2 | GAATGTCAAGTTGTATTGGATGGTTATGCCATTGTTGAAGTCGCAGGATACTGCC | 598 |
| LC-GNC2A | antisense/F3 | CCAAGTCTAACTATGGCTTGTATGGCCAAACAGTCACAGACTCCGCTCAATGACG | 599 |

Junin virus

| | | | |
|---|---|---|---|
| SLA | antisense/F4 | AGCTGATGACCAGGTTAGTTGAAGACTTCTCAGAAGCTGTGGGTAGTTCAATGAG | 600 |
| PLMGA | antisense/F5 | TGATAAGAATTCATGGAAGCACACCATTTCCAGCAGTTCTGTTCTGTCTTGACACT | 601 |

Machupo virus

| | | | |
|---|---|---|---|
| MPLMG1A | antisense/F6 | TTAGATGACAGAAACTCATGGAAGCAGATCATCTCGAGCCACTCCGCAGCATCTG | 602 |
| MPLMG2A | antisense/F7 | TAAGGCCACAGTTGGTGCAGCATAGAAACAACACTCTCATCATCTTCTAGGGCCC | 603 |

Guanarito virus

| | | | |
|---|---|---|---|
| GV1A | antisense/F8 | CTGAAGCAGTGTACATAGTTTCCTGAAGAAGGCTGAAACACTGCCAACTCCACGG | 604 |
| GV2A | antisense/F9 | TTCTTCTTGGGAACAATGACACCATTGTGTGCTCTGCAGAGATGGCCAAATTT | 605 |
| GSSA | antisense/F10 | CATGTCCCAATCCCTTAATTGTCCACCCAAGAATCCAATCAAACTCTGGAGAGCC | 606 |
| GNPA | antisense/F11 | GACTGAAACATGATGCAATCGAGAAGAGCACAGTGTGGCTCCTTCGTGGAGTTAG | 607 |

Crimean-Congo Hemorrhagic Fever virus

| | | | |
|---|---|---|---|
| CCVA | antisense/F12 | GTTGGTTCGGTGTGTCCGTAGGTTAAGAATGGACTTGGTGTGTCCAGATCCC | 608 |

Hantavirus

| | | | |
|---|---|---|---|
| MSS1A | antisense/G1 | CCAGCTTGGCTCCAAGACAGTTTCTGATGCACTAGCTGCCCACATGATTGATTC | 609 |
| MSS2A | antisense/G2 | CTGCCTACAGGTTTAAGTTGGTCAATGTATAGTCCGCATGCAGTACAGCCCGTAC | 610 |
| G1/G2A | antisense/G3 | GTTGAGCCACTATAACCACCCTTCCCTGACACTTTGACTGTATTCTGGCCTCTTG | 611 |
| MSPG-A | antisense/G4 | GATCAGTATGCATTGGGACAACACCAACACCATGTGCGTTGTCATTCCAGCTTGG | 612 |

Rift Valley Fever virus

| | | | |
|---|---|---|---|
| LG2-1A | antisense/G5 | GGCTTGGCAGAGAGTTCTCTCTTCACAACCACTCTCTCATACCCTACACATAAAGGC | 613 |
| LG2-2A | antisense/G6 | CCTTCTTTGAGTTCTTAAACACTGCAAAGGGCACAACCTCATGTGCAGTGCTGAG | 614 |

Dengue virus

| | | | |
|---|---|---|---|
| NCR1A | antisense/G7 | ACAGATAGCATTAGCAGCTAGTCTCAGGTCTCTCCTGTGGAAGTACATCAGCTGC | 615 |
| NCR2A | antisense/G8 | GCAGGATCTCTGGTCTCTCCCAGCGTCAATATGCTGTTTATTGTTATGCCGG | 616 |
| NCR3A | antisense/G9 | CCGTCAAAGCACCATCTTCTGTCAGAGTACTGGAAGCCTTCTGAGGCAACTTTG | 617 |
| NCR4A | antisense/G10 | GACAAATGTCAAGGACAGCAAGGTAGCCCACAGCTGATGTGACTGAAAGTCAGTC | 618 |
| NCR5A | antisense/G11 | CGCGTCTTGTTCTTCCACCAGTGTAGCTTCTCCTTGTGTTGGACATCTTGAATCG | 619 |

Yersinia pestis

| | | | |
|---|---|---|---|
| rpoB1A | antisense/G12 | CCGCTTCCAGGATCTGCAACTCTTCAGTCAGGTCTTTCTTAGCCTGTTTCAGTTG | 620 |
| rpoB2A | antisense/H1 | CGGATCAAACTCGAAATCTAACCATGAACCGCGGTAAGGGATGATACGTGCGTTG | 621 |

West Nile virus

| | | | |
|---|---|---|---|
| WNV1A | antisense/H2 | CTCTGTTGTCATCCACTCTCCTCCTGCATGGATGGACCACGTGGTTCTTC | 622 |
| WNV2A | antisense/H3 | CACAGACACAAATGGATTCACGGTCACCAGTCTTCCAACAGGTGTGAGGTCAT | 623 |
| WNV3A | antisense/H4 | CCGTCGAGGGTTTCCATTCCTGGATGTCTTTGCGGACCTTTGACATAGCATTT | 624 |

SARS-CoV

| | | | |
|---|---|---|---|
| SGPA | antisense/H5 | GTGAAGTTCCTCTCCTGGGATGGCACATACGTGACATGTAGGAAGACAACACCATG | 625 |
| ORF 1aA | antisense/H6 | AGCTACACTCCACTCATCAAGATCAATACCCATGTTGGTAAGGAGATCAGAAACTGGT | 626 |
| SMPA | antisense/H7 | CGAGCAAACAGCCTGAAGGAAGCAACGAAGTAGCTAAGCCACATCAAGCCTACAATAC | 627 |
| NP1A | antisense/H8 | CCAATGTGGTCTTTGGGTGTATTCAAGGCTCCCTCAGTTGCAACCCATACGATG | 628 |
| EPA | antisense/H9 | GAAGCGCAGTAAGGATGGCTAGTGTGACTAGCAAGAATACCACGAAAGCAAGAAA | 629 |
| SSA | antisense/H10 | AATTGGATCCATTGCTGGATTAGCAACTCCTGAAGAGCCGTCGATTGTGTGTATTT | 630 |
| SARs1A | antisense/H11 | ACCCATCATAGAGATGAGTCTACGGTAGGTCATGTCCTTTGGTATGCCTGGTATGTCA | 631 |
| SARs2A | antisense/H12 | TGATGTTCCACCTGGTTTAACATATAGTGAGCCGCCACACATGACCATCTCACTT | 632 |

Hybridization of Dye-Conjugated RT-PCR Products With the Microarray

Eluted dye-conjugated amplified pathogen-specific genomic sequences were vacuum-dried down and eluted in 9.5 μl of sterile double-distilled water. To the eluate, 2 μg of poly A, 1 μl of yeast tRNA, 2.25 μl of 20% SCC, and 0.25 μl of 10% SDS was added. The mixture was heated at 98° C. for 2 minutes, snap cooled on wet ice and centrifuged for 5 minutes. The oligonucleotide microarrays were prehybridized with prehybridization buffer (5×SSC, 0.1% SDS and 1% BSA) at 42° C. for 45 minutes and then dried at 800 rpm for 2 minutes. The 4 μl samples were then spotted onto the oligonucleotide microarrays. The slides were covered with a cover slip and incubated for 30 minutes at 65° C. on a water bath.

The slides were washed sequentially with 1×SSC, 0.1% SDS for 10 minutes at room-temperature and 0.1×SSC for 10 minutes at room-temperature, then dried by centrifuging at 800 rpm for 2 minutes.

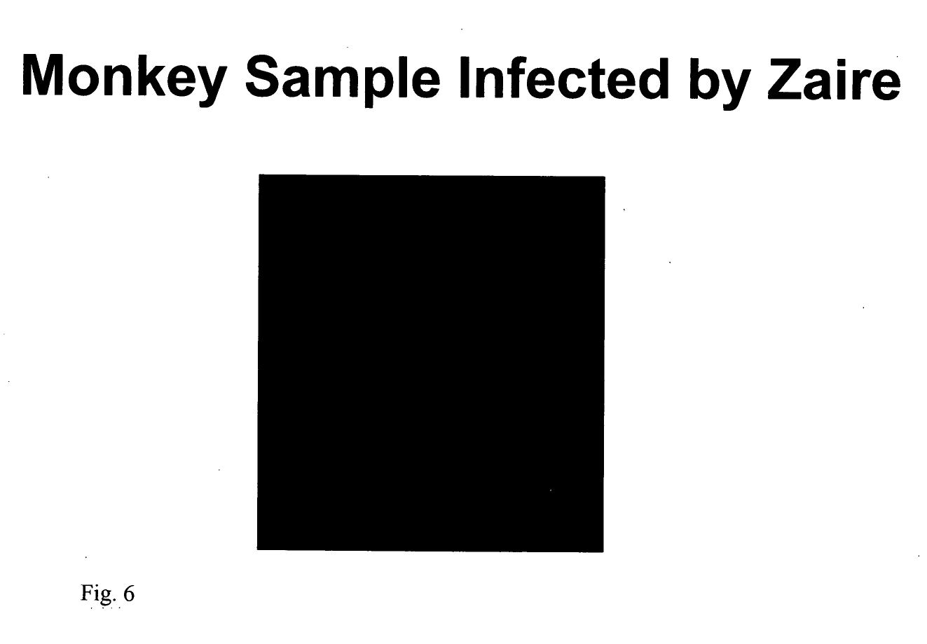

The slides were scanned on a GenePix 4000A scanner (Axon, Foster City, Calif.) at 10 μm resolution to capture tif images (FIG. 6). The PMT voltage was 680 for detection at 635 nm and 700 for detection at 535 nm.

### Example 3

### Amplification and Detection of Pseudomonas aeruginosa-Specific Nucleic Acids in a Sample

This example demonstrates that Pseudomonas aeruginosa-specific nucleic acids can be amplified from a sample and detected using specific probes on an oligonucleotide array.

Production of Primers and Probes

The P. aeruginosa genome was screened for bacterial-specific sequences. These sequences were blasted against the human genome to ensure that no highly similar sequences are present in the human genome. On the basis of this analysis, P. aeruginosa-specific oligonucleotide probes were designed using Primer Quest software (Integrated DNA Technologies, Inc., Coralville, Iowa), that correspond to the target Pseudomonas aeruginosa-specific genomic sequences (each about 55 base pairs in length, with a Tₘ of 72-73° C. and a percent GC content of 45-50). Both sense and antisense versions of each P. aeruginosa-specific oligonucleotide probe were prepared. Primers with sequences that flank those of the target P. aeruginosa-specific genomic sequences were also prepared (each about 23 base pairs in length, with a Tₘ of 55° C.). All primers and probes were synthesized by Qiagen Operon (Alameda, Calif.) and were dissolved in DEPC treated H₂O at a concentration of 1 μg/μl.

PCR Amplification

PCR amplification was performed directly on a 5 μl sample of blood spiked with one or more P. aeruginosa bacteria in a 25 μl volume containing 20 μl of PCR mix: 2.5 μl of 10× reaction buffer (Invitrogen, Carlsbad, Calif.), 0.125 μl of blood-resistant DNA polymerase (HemoKlentaq, DNA Polymerase Technology, Sausalito, Calif.), 150 μM of each of dATP, dGTP and dCTP, 120 μM of dTTP, 60 μM of amino-allyl dUTP (Sigma, St. Louis, Mo.), 0.20 to 0.50 μM of each forward primer (SEQ ID NOs: 633-639; Table 7), 0.20 to 0.50 μM of each reverse primer (SEQ ID NOs: 640-646; Table 7), and sterile double-distilled water.

The PCR thermocycling program consisted of one cycle of 2 minutes at 96° C.; forty cycles of 95° C. for 30 seconds, 55° C. for 30 seconds, and 72° C. for 15 seconds (amplification); and one cycle of 15 seconds at 72° C. (final extension).

TABLE 7

| | | | SEQ |
|---|---|---|---|
| Gene | | | ID |
| Name | Direction | Primer | NO: |
| EXOa1 | forward | ATCGACAACGCCCTCAGCATCA | 633 |
| OPRD | forward | CCAATCGCTGGGCTTCGATTTCAA | 634 |
| OPRI | forward | TTGCAGCAGCCACTCCAAAGAAAC | 635 |
| LipA | forward | AGCACCTACACCCAGACCAAATAC | 636 |
| aaCA4 | forward | TATACAAATGCCTGGAGCGGAGCA | 637 |
| EXOa2 | forward | ACGATACCTGGGAAGGCAAGATCTAC | 638 |
| Aada1 | forward | ATCAGCGCACTAGATGGCTCAGAA | 639 |
| EXOa1 | reverse | CGTTCAGTTCGTGGATGAACACCT | 640 |
| OPRD | reverse | TCTGGTAGGCCAAGGTGAAAGTGT | 641 |
| OPRI | reverse | TCGTGAGACCTATTACTTGCGGCT | 642 |
| LipA | reverse | CGACGATTTCCTCCACCTGTTGC | 643 |
| aaCA4 | reverse | TCAATGTTTCTCGATGCAAGCGCC | 644 |
| EXOa2 | reverse | TGGCGATGACGGGTGAAAGTCT | 645 |
| Aada1 | reverse | AATCGTCAGGCGAGATCGAATGGA | 646 |

Purification of PCR Products and Dye Conjugation

Following the PCR, amplified P. aeruginosa-specific genomic sequences were purified with the QiaQuick PCR Purification Kit (Qiagen, Valencia, Calif.) according to the manufacturer's instructions, vacuum-dried and eluted in 9 μl of sterile double-distilled water. To the eluate, 1 μl of 1M sodium bicarbonate buffer pH 9.0 was added, followed by 4.5 μl NHS-cye dye (Cy3 or Cy5, Pharmacia, Piscataway, N.J.). The conjugation mixture was then incubated at room-temperature for one hour in the dark and quenched with 4 M hydroxylamine.

To remove unincorporated cye dyes, the conjugation mixture was purified with the QiaQuick PCR Purification Kit (Qiagen, Valencia, Calif.) according to the manufacturer's instructions, and Cy3- and CyS-labeled products were combined. To the combined Cy3- and CyS-labeled products, 60 μl of sterile double-distilled water was added, followed by 500 μl of PB buffer (Qiagen, Valencia, Calif.). The mixture was applied to a QiaQuick column and spun at 13,000 rpm for 1 minute. The flow-through was reloaded onto the same column for a second spin and then discarded. The column was washed twice with 500 μl of PE buffer (Qiagen, Valencia, Calif.) and spun at 13,000 rpm for 1 minute. Dye-conjugated amplified P. aeruginosa-specific genomic sequences were eluted from the column with 20 μl of EB buffer (Qiagen, Valencia, Calif.) for 1 minute room-temperature, followed by centrifugation at 13,000 rpm for 1 minute. The elution step was repeated two additional times.

Production of Oligonucleotide Microarrays

Oligonucleotide probes (SEQ ID NOs: 647-660; Table 8) were solubilized in 50% DMSO and spotted in duplicate on poly-L-lysine coated slides or Ultra GAPS slides (Coming, Acton, Mass.) using an OmniGrid arrayer (Gene Machines, San Carlos, Calif.) at a concentration of 50 μM. Slides were processed for hybridization according to Xiang and Brownstein (Fabrication of cDNA microarrays, in: Methods in Molecular Biology, vol. 224, Functional Genomics, Methods and Protocols, M. J. Brownstein and A. Khodursky (eds.), Humana Press Inc., 2003).

TABLE 8

| | | | SEQ |
|---|---|---|---|
| Gene | ID | | |
| Name | Direction | Oligonucleotide | NO: |
| EXOa1 | sense | CAGTTGGTCGCTGAACTGGCTGGTACCGATCGGCCACGAGAAGCCCTCGAACAT | 647 |
| OPRD | sense | CATCTACCGCACAAACGATGAAGGCAAGGCCAAGGCCGGCGACATCAGCAACACCACTTG | 648 |
| OPRI | sense | GAAGCCTATCGCAAGGCTGACGAAGCTCTGGGCGCTGCTCAGAAAGCTCAGCAGACTG | 649 |
| LipA | sense | TGACGGTGCCCAGGTCTACGTCACCGAAGTCAGCCAGTTGGACACCTCGGAAGTC | 650 |
| aaCA4 | sense | ACATGGCGACGCCTGTCCCGAGAACTTCATCTTCCAAGGTAATGCCTTCGTCGGCTT | 651 |
| EXOa2 | sense | AAGCATGACCTGGACATCAAACCCACGGTCATCAGTCATCGCCTGCACTTTCCCGA | 652 |
| Aada1 | sense | TTGATTGGCCACAATGCCGCTGACGCCGCAGATGCAGATCCAGTTATTGTTGTTGCA | 653 |
| EXOa1 | antisense | ATGTTCGAGGGCTTCTCGTGGCCGATCGGTACCAGCCAGTTCAGCGACCAACTG | 654 |
| OPRD | antisense | CAAGTGGTGTTGCTGATGTCGCCGGCCTTGGCCTTGCCTTCATCGTTTGTGCGGTAGATG | 655 |
| OPRI | antisense | CAGTCTGCTGAGCTTTCTGAGCAGCGCCCAGAGCTTCGTCAGCCTTGCGATAGGCTTC | 656 |
| LipA | antisense | GACTTCCGAGGTGTCCAACTGGCTGACTTCGGTGACGTAGACCTGGGCACCGTCA | 657 |
| aaCA4 | antisense | AAGCCGACGAAGGCATTACCTTGGAAGATGAAGTTCTCGGGACAGGCGTCGCCATGT | 658 |
| EXOa2 | antisense | TCGGGAAAGTGCAGGCGATGACTGATGACCGTGGGTTTGATGTCCAGGTCATGCTT | 659 |
| Aada1 | antisense | TGCAACAACAATAACTGGATCTGCATCTGCGGCGTCAGCGGCATTGTGGCCAATCAA | 660 |

Hybridization of Dye-Conjugated RT-PCR Products With the Microarray

Eluted dye-conjugated amplified P. aeruginosa-specific genomic sequences were vacuum-dried down and eluted in 9.5 μl of sterile double-distilled water. To the eluate, 2 μl of poly A, 1 μl of yeast tRNA, 2.25 μl of 20% SCC, and 0.25 μl of 10% SDS was added. The mixture was heated at 98° C. for 2 minutes, snap cooled on wet ice and centrifuged for 5 minutes. The oligonucleotide microarrays were prehybridized with prehybridization buffer (5×SSC, 0.1% SDS and 1% BSA) at 42° C. for 45 minutes and then dried at 800 rpm for 2 minutes. The 4 μl denatured samples were then spotted onto the oligonucleotide microarrays. The slides were covered with a cover slip and incubated for 30 minutes at 65° C. on a water bath.

The slides were washed sequentially with 1×SSC, 0.1% SDS for 10 minutes at room-temperature and 0.1×SSC for 10 minutes at room-temperature, then dried by centrifuging at 800 rpm for 2 minutes.

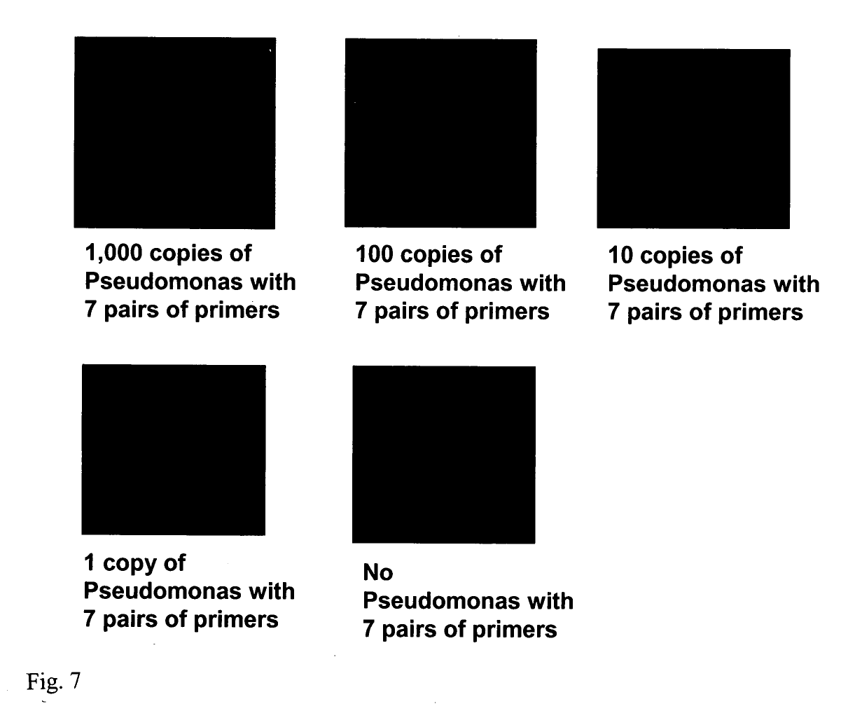

The slides were scanned on a GenePix 4000A scanner (Axon, Foster City, Calif.) at 10 μm resolution to capture tif images (FIG. 7). The PMT voltage was 680 for detection at 635 nm and 700 for detection at 535 nm.

### Example 4

### Amplification and Detection of HIV-based Retroviral Vector-Specific Nucleic Acids in a Sample

This example demonstrates that HIV-based retroviral vector-specific nucleic acids can be amplified from a sample and detected using specific probes on an oligonucleotide array.

Production of Primers and Probes

An HIV-based retroviral vector was screened for vector-specific sequences. These sequences were blasted against the human genome to ensure that no highly similar sequences are present in the human genome. On the basis of this analysis, HIV-based retroviral vector-specific oligonucleotide probes were designed using Primer Quest software (Integrated DNA Technologies, Inc., Coralville, Iowa), that correspond to the target HIV-based retroviral vector-specific sequences (each about 55 base pairs in length, with a Tₘ of 72-73° C. and a percent GC content of 45-50). Both sense and antisense versions of each HIV-based retroviral vector-specific oligonucleotide probe were prepared. Primers with sequences that flank those of the target HIV-based retroviral vector-specific sequences were also prepared (each about 23 base pairs in length, with a Tₘ of 55° C.). All primers and probes were synthesized by Qiagen Operon (Alameda, Calif.) and were dissolved in DEPC treated H₂O at a concentration of 1 μg/μl.

PCR Amplification

PCR amplification was performed directly on a 5 μl sample of blood spiked with one or more HIV-based retroviral vector copies in a 25 μl volume containing 20 μl of PCR mix: 2.5 μl of 10× reaction buffer (Invitrogen, Carlsbad, Calif.), 0.125 μl of blood-resistant DNA polymerase (HemoKlentaq, DNA Polymerase Technology, Sausalito, Calif.), 150 μM of each of dATP, dGTP and dCTP, 120 μM of dTTP, 60 μM of amino-allyl dUTP (Sigma, St. Louis, Mo.), 0.20 to 0.50 μM of each forward primer (SEQ ID NOs: 661-666; Table 9), 0.20 to 0.50 μM of each reverse primer (SEQ ID NOs: 667-672; Table 9), and sterile double-distilled water.

The PCR thermocycling program consisted of one cycle of 2 minutes at 96° C.; forty cycles of 95° C. for 30 seconds, 55° C. for 30 seconds, and 72° C. for 15 seconds (amplification); and one cycle of 15 seconds at 72° C. (final extension).

TABLE 9

| | | | SEQ |
|---|---|---|---|
| Gene | | | ID |
| Name | Direction | Primer | NO: |
| cmv-bac-1F | forward | AGCGACCTCCACCAGACATTGAAA | 661 |
| env-1F | forward | AGGCAAAGAGAAGAGTGGTGCAGA | 662 |
| icfP-1F | forward | TGACCCTGAAGTTCATCTGCACCA | 663 |
| gag-1F | forward | ATTGGACGAACCACTGAATTGCCG | 664 |
| lba-1F | forward | TGTAACTCGCCTTGATCGTTGGGA | 665 |
| LacZF | forward | AGGCCACCACTTCAAGAACTCTGT | 666 |
| cmv-bac-1R | reverse | GCTGCTTGCTTTGTTCAAACTGCC | 667 |
| env-1R | reverse | CCCTCAGCAAATTGTTCTGCTGCT | 668 |
| icfP-1R | reverse | TCTTGTAGTTGCCGTCGTCCTTGA | 669 |
| gag-1R | reverse | AACAGACGGGCACACACTACTTGA | 670 |
| lba-1R | reverse | TCCTGCAACTTTATCCGCCTCCAT | 671 |
| LacZR | reverse | ATCGTCTTGAGTCCAACCCGGTAA | 672 |

Purification of PCR Products and Dye Conjugation

Following the PCR, amplified HIV-based retroviral vector-specific sequences were purified with the QiaQuick PCR Purification Kit (Qiagen, Valencia, Calif.) according to the manufacturer's instructions, vacuum-dried and eluted in 9 μl of sterile double-distilled water. To the eluate, 1 μl of 1M sodium bicarbonate buffer pH 9.0 was added, followed by 4.5 μl NHS-cye dye (Cy3 or Cy5, Pharmacia, Piscataway, N.J.). The conjugation mixture was then incubated at room-temperature for one hour in the dark and quenched with 4 M hydroxylamine.

To remove unincorporated cye dyes, the conjugation mixture was purified with the QiaQuick PCR Purification Kit (Qiagen, Valencia, Calif.) according to the manufacturer's instructions, and Cy3- and Cy5-labeled products were combined. To the combined Cy3- and Cy5-labeled products, 60 μl of sterile double-distilled water was added, followed by 500 μl of PB buffer (Qiagen, Valencia, Calif.). The mixture was applied to a QiaQuick column and spun at 13,000 rpm for 1 minute. The flow-through was reloaded onto the same column for a second spin and then discarded. The column was washed twice with 500 μl of PE buffer (Qiagen, Valencia, Calif.) and spun at 13,000 rpm for 1 minute. Dye-conjugated amplified HIV-based retroviral vector-specific sequences were eluted from the column with 20 μl of EB buffer (Qiagen, Valencia, Calif.) for 1 minute room-temperature, followed by centrifugation at 13,000 rpm for 1 minute. The elution step was repeated two additional times.

Production of Oligonucleotide Microarrays

Oligonucleotide probes (SEQ ID NOs: 673-684; Table 10) were solubilized in 50% DMSO and spotted in duplicate on poly-L-lysine coated slides or Ultra GAPS slides (Coming, Acton, Mass.) using an OmniGrid arrayer (Gene Machines, San Carlos, Calif.) at a concentration of 50 μM. Slides were processed for hybridization according to Xiang and Brownstein (Fabrication of cDNA microarrays, in: Methods in Molecular Biology, vol. 224, Functional Genomics, Methods and Protocols, M. J. Brownstein and A. Khodursky (eds.), Humana Press Inc., 2003).

TABLE 10

| | | | SEQ |
|---|---|---|---|
| Gene | | | ID |
| Name | Direction | Oligonucleotide | NO: |
| cmv-bac-1 | sense | AACCTGGACGCTTTATGGGATTGTCTGACCGGATGGGTGGAGTACCCGCTCGTTT | 673 |
| env-1 | sense | TTTGTTCCTTGGGTTCTTGGGAGCAGCAGGAAGCACTATGGGCGCAGCCTCAATGA | 674 |
| icfP-1 | sense | ACATGAAGCAGCACGACTTCTTCAAGTCCGCCATGCCCGAAGGCTACGTCCAGGAG | 675 |
| gag-1 | sense | ACCAGATCTGAGCCTGGGAGCTCTCTGGCTAACTAGGGAACCCACTGCTTAAGCCT | 676 |
| lba-1 | sense | AAACGACGAGCGTGACACCACGATGCCTGTAGCAATGGCAACAACGTTGCGCAAACT | 677 |
| LacZ | sense | ACCTCGCTCTGCTAATCCTGTTACCAGTGGCTGCTGCCAGTGGCGATAAGTCGTGT | 678 |
| cmv-bac-1 | antisense | AAACGAGCGGGTACTCCACCCATCCGGTCAGACAATCCCATAAAGCGTCCAGGTT | 679 |
| env-1 | antisense | TCATTGAGGCTGCGCCCATAGTGCTTCCTGCTGCTCCCAAGAACCCAAGGAACAAA | 680 |
| icfP-1 | antisense | CTCCTGGACGTAGCCTTCGGGCATGGCGGACTTGAAGAAGTCGTGCTGCTTCATGT | 681 |
| gag-1 | antisense | AGGCTTAAGCAGTGGGTTCCCTAGTTAGCCAGAGAGCTCCCAGGCTCAGATCTGGT | 682 |
| lba-1 | antisense | AGTTTGCGCAACGTTGTTGCCATTGCTACAGGCATCGTGGTGTCACGCTCGTCGTTT | 683 |
| LacZ | antisense | ACACGACTTATCGCCACTGGCAGCAGCCACTGGTAACAGGATTAGCAGAGCGAGGT | 684 |

Hybridization of Dye-Conjugated RT-PCR Products With the Microarray

Eluted dye-conjugated amplified HIV-based retroviral vector-specific sequences were vacuum-dried down and eluted in 9.5 μl of sterile double-distilled water. To the eluate, 2 μl of poly A, 1 μl of yeast tRNA, 2.25 μl of 20% SCC, and 0.25 μl of 10% SDS was added. The mixture was heated at 98° C. for 2 minutes, snap cooled on wet ice and centrifuged for 5 minutes. The oligonucleotide microarrays were prehybridized with prehybridization buffer (5×SSC, 0.1% SDS and 1% BSA) at 42° C. for 45 minutes and then dried at 800 rpm for 2 minutes. The 4 μl denatured samples were then spotted onto the oligonucleotide microarrays. The slides were covered with a cover slip and incubated for 30 minutes at 65° C. on a water bath.

The slides were washed sequentially with 1×SSC, 0.1% SDS for 10 minutes at room-temperature and 0.1×SSC for 10 minutes at room-temperature, then dried by centrifuging at 800 rpm for 2 minutes.

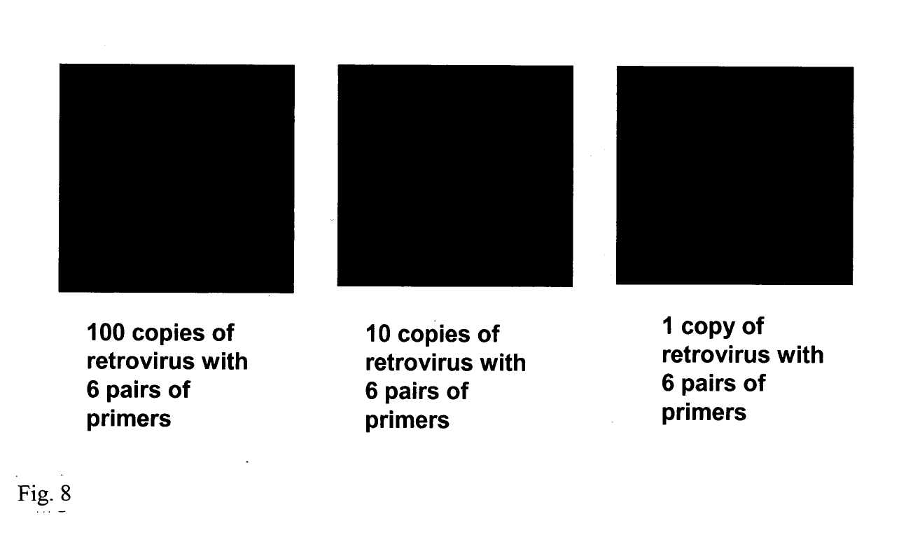

The slides were scanned on a GenePix 4000A scanner (Axon, Foster City, Calif.) at 10 μm resolution to capture tif images (FIG. 8). The PMT voltage was 680 for detection at 635 nm and 700 for detection at 535 nm.

While this disclosure has been described with an emphasis upon preferred embodiments, it will be obvious to those of ordinary skill in the art that variations of the preferred embodiments may be used and it is intended that the disclosure may be practiced otherwise than as specifically described herein. Accordingly, this disclosure includes all modifications encompassed within the spirit and scope of the disclosure as defined by the claims below.

We claim:

1. An oligonucleotide array, comprising a plurality of single-stranded nucleic acid probe pairs affixed at discrete addressable locations on a solid support, wherein each of the probe pairs comprises:
(a) an antisense nucleic acid probe sequence specifically complementary to the sense strand of a double-stranded target nucleic acid, and
(b) a sense nucleic acid probe sequence specifically complementary to the antisense strand of the double-stranded target nucleic acid.

2. The array according to claim 1, wherein each antisense nucleic acid probe sequence of a probe pair consists essentially of the complement of the corresponding sense nucleic acid probe sequence.

3. The array according to claim 1, wherein each antisense nucleic acid probe sequence of a probe pair consists essentially of at least one of the sequences shown in SEQ ID NOs: 441-536 and each sense nucleic acid probe sequence of the probe pair consists essentially of at least one of the sequences shown in SEQ ID NOs: 537-632.

4. The array according to claim 1, wherein the number of locations on the array is from about 50 to about 1,000.

5. The array according to claim 1, wherein the solid support is flexible.

6. The array according to claim 5, wherein the solid support comprises nylon.

7. The array according to claim 1, wherein the solid support is rigid.

8. The array according to claim 7, wherein the solid support comprises glass.

9. A method for detecting target nucleic acids in a sample, comprising:
(a) extracting total nucleic acid from the sample;
(b) hybridizing a plurality of target-specific primers to the total nucleic acid;
(c) amplifying target-specific nucleic acids from the total nucleic acid utilizing the target-specific primers to produce amplified target-specific nucleic acid molecules;
(d) contacting the amplified target-specific nucleic acid molecules with the array according to claim 1 under conditions sufficient to produce a hybridization pattern;
(e) detecting the hybridization pattern; and
(f) identifying the target nucleic acids in the sample based on the hybridization pattern.

10. The method according to claim 9, further comprising reverse transcribing a plurality of target-specific cDNAs complementary with target transcripts contained in the total nucleic acid prior to amplifying target-specific DNAs and cDNAs.

11. The method according to claim 10, wherein the target nucleic acids comprise one or more nucleic acids from one or more pathogens.

12. The method according to claim 10, wherein the pathogens comprise Variola major, Vaccinia virus, Ebola virus, Marburg virus, Bacillus anthracis, Clostridium botulinum, Francisella tularensis, Lassa Fever virus, Lymphocytic Choriomeningitis virus, Junin virus, Machupo virus, Guanarito virus, Crimean-Congo Hemorrhagic Fever virus, Hantavirus, Rift Valley Fever virus, Dengue virus, Yersinia pestis, West Nile virus, SARS-CoV, or combinations of two or more thereof.

13. The method according to claim 11, wherein the plurality of target-specific primers are selected from the group listed in Table 5.

14. The method according to claim 9, wherein the amplification utilizes polymerase chain reaction.

15. The method according to claim 9, wherein the amplified targets are labeled targets.

16. The method according to claim 9, wherein the amplified targets comprise an amino-allyl dNTP.

17. The method according to claim 16, further comprising conjugating a detectable label to the amino-allyl dNTP prior to hybridizing the amplified target-specific nucleic acid molecules to the array.

18. The method according to claim 17, wherein the detectable label comprises a fluorescent dye or biotin.

19. The method according to claim 9, wherein the method further comprises washing the array prior to detecting the hybridization pattern.

20. A kit for use in identifying a pathogen in a sample, comprising:
the array according to claim 1.

21. The kit according to claim 20, further comprising one or more reagents for generating a labeled target.

22. The kit according to claim 20, further comprising a hybridization buffer.

23. The kit according to claim 20, further comprising a wash medium.
